SEQUENCE LISTING (24 sequences)

SEQ ID NO: 1 - DNA, 61 bp, synthetic construct
  source 1..61: mol_type = other DNA
ctgataagctaagataccctaaccatcgatcgtcgccgtccagctcgacctcaacatcagt

SEQ ID NO: 2 - DNA, 65 bp, synthetic construct
  source 1..65: mol_type = other DNA
ctgataagctaagataccctaaccatcgattttacgtcgccgtccagctcgacctcaacatcagt

SEQ ID NO: 3 - DNA, 61 bp, synthetic construct
  source 1..61: mol_type = other DNA
ctgataagctacgtcgccgtccagctcgaccagataccctaaccatcgattcaacatcagt

SEQ ID NO: 4 - DNA, 61 bp, synthetic construct
  source 1..61: mol_type = other DNA
ctgataagctaagataccctcgtcgccgtccagctcgaccaaccatcgattcaacatcagt

SEQ ID NO: 5 - DNA, 61 bp, synthetic construct
  source 1..61: mol_type = other DNA
ctgataagctaatcgaccctaaccatcgatcgtcgccgtccagctcgacctcaacatcagt

SEQ ID NO: 6 - DNA, 61 bp, synthetic construct
  source 1..61: mol_type = other DNA
ctgataagctaatcgatggtaaccatcgatcgtcgccgtccagctcgacctcaacatcagt

SEQ ID NO: 7 - DNA, 62 bp, synthetic construct
  source 1..62: mol_type = other DNA
ctgataagctaggtcgaggctgaccatcgatcgtcgccgtccagctcgacctcaacatcagt

SEQ ID NO: 8 - DNA, 61 bp, synthetic construct
  source 1..61: mol_type = other DNA
ctgataagctaggtcgagctggacggcgaccgtcgccgtccagctcgacctcaacatcagt

SEQ ID NO: 9 - DNA, 61 bp, synthetic construct; identical to SEQ ID NO: 1
  source 1..61: mol_type = other DNA

SEQ ID NO: 10 - DNA, 61 bp, synthetic construct
  source 1..61: mol_type = other DNA
gaaactacctaagataccctaaccatcgatcgtcgccgtccagctcgacccccaacaacat

SEQ ID NO: 11 - DNA, 61 bp, synthetic construct
  source 1..61: mol_type = other DNA
ggtaacggtttagataccctaaccatcgatcgtcgccgtccagctcgaccaactcagtaat

SEQ ID NO: 12 - DNA, 58 bp, synthetic construct
  source 1..58: mol_type = other DNA
aaatccattagataccctaaccatcgatcgtcgccgtccagctcgacccctgctccaa

SEQ ID NO: 13 - DNA, 61 bp, synthetic construct
  source 1..61: mol_type = other DNA
ggtcgagctggacggcgacgatcgatggttagggtatcttagcttatcagactgatgttga

SEQ ID NO: 14 - RNA, 22 nt, synthetic construct
  source 1..22: mol_type = other RNA
tagcttatcagactgatgttga

SEQ ID NO: 15 - RNA, 22 nt, synthetic construct
  source 1..22: mol_type = other RNA
tagcttatcatactgatgttga

SEQ ID NO: 16 - RNA, 23 nt, synthetic construct
  source 1..23: mol_type = other RNA
caaagtgcttacagtgcaggtag

SEQ ID NO: 17 - RNA, 23 nt, synthetic construct
  source 1..23: mol_type = other RNA
taaggtgcatctagtgcagatag

SEQ ID NO: 18 - RNA, 23 nt, synthetic construct
  source 1..23: mol_type = other RNA
aaaagtgcttacagtgcaggtag

SEQ ID NO: 19 - RNA, 23 nt, synthetic construct
  source 1..23: mol_type = other RNA
gtccagttttcccaggaatccct

SEQ ID NO: 20 - RNA, 22 nt, synthetic construct
  source 1..22: mol_type = other RNA
taggtagtttcatgttgttggg

SEQ ID NO: 21 - RNA, 22 nt, synthetic construct
  source 1..22: mol_type = other RNA
tccttcattccaccggagtctg

SEQ ID NO: 22 - RNA, 22 nt, synthetic construct
  source 1..22: mol_type = other RNA
aaaccgttaccattactgagtt

SEQ ID NO: 23 - RNA, 19 nt, synthetic construct
  source 1..19: mol_type = other RNA
aatggatttttggagcagg

SEQ ID NO: 24 - RNA, 41 nt, synthetic construct
  source 1..41: mol_type = other RNA
taatttctactaagtgtagatcgtcgccgtccagctcgacc

(19) United States
(12) Patent Application Publication
(10) Pub. No.: US 2025/0129410 A1
(43) Pub. Date: Apr. 24, 2025

(54) ONE-POT ENDONUCLEOLYTICALLY EXPONENTIATED ROLLING CIRCLE AMPLIFICATION BY CRISPR-CAS12a

(71) Applicant: University of Florida Research Foundation, Inc., Gainesville, FL (US)

(72) Inventors: He Yan, Gainesville, FL (US); Yong Zeng, Gainesville, FL (US)

(21) Appl. No.: 18/837,232

(22) PCT Filed: Feb. 27, 2023
(86) PCT No.: PCT/US2023/063351

§ 371 Date: Aug. 9, 2024

Related U.S. Application Data
(60) Provisional application No. 63/313,870, filed on Feb. 25, 2022.

(51) Int. Cl. C12Q 1/682

(52) U.S. Cl. CPC C12Q 1/682

(57) ABSTRACT

Described herein are methods, compositions, and kits relating to the mixed detection of one or more polynucleotides, in particular miRNAs. In certain aspects, methods, systems, compositions, and kits utilize Cas12a for polynucleotide detection, in particular miRNAs. In aspects the methods, systems, compositions, and kits utilize Cas12a for detection of polynucleotide targets in a single pot reaction.

### RELATED APPLICATION

This application claims the benefit of U.S. Provisional Application Ser. No. 63/313,870, titled “ONE-POT ENDONUCLEOLYTICALLY EXPONENTIATED ROLLING CIRCLE AMPLIFICATION BY CRISPR-CAS12a,” filed Feb. 25, 202. This application is incorporated herein by reference in its entirety.

### STATEMENT REGARDING FEDERALLY SPONSORED RESEARCH OR DEVELOPMENT

This invention was made with government support under Grant Numbers: R33 CA214333, R01 CA243445, R33 CA252158 and R01 CA260132, awarded by the National Institutes of Health. The government has certain rights in the invention.

### SEQUENCE LISTING

The instant application contains a Sequence Listing filed in ST.26 format entitled 222111_2880SequenceListing.xml created on Feb. 27, 2022. The content of the sequence listing is incorporated herein in its entirety.

### FIELD

The present disclosure relates to CRISPR/Cas complex-based systems and methods.

### BACKGROUND

MicroRNAs (miRNAs) are endogenous and short non-coding single-stranded RNAs (18-23 nucleotides) that are involved in the post-transcriptional repression of messenger RNAs (mRNAs). Because they participate in various biological processes such as cell proliferation, differentiation, and cell death, dysregulated miRNAs are closely linked to the pathogenesis of diseases such as cancers. miRNAs were originally studied in tissues, but they have also been discovered in the blood, urine, and other body fluids, either associated with ribonucleoprotein complexes or argonaute-2, or encapsulated in exosomes, making the detection of circulating miRNAs a promising strategy in liquid biopsy for cancer diagnosis, prognosis, and monitoring. Accordingly, the rising applications of miRNA biomarkers accelerate the development of novel technologies for the detection of miRNAs. However, miRNA detection remains challenging due to the short length, high sequence similarity within multiple members of miRNA families, and wide concentration range in different cell types. So, approaches that fulfill the rapid, accurate, and highly sensitive detection of miRNAs with simple steps, especially the one-pot method, are expected.

Reverse transcription-quantitative polymerase chain reaction (RT-qPCR) has been the gold standard approach for the miRNA detection thus far, but in comparison to the traditional RT-qPCR for mRNA analysis, the short length of miRNAs necessitates a special RT process to incorporate extended sequences that facilitate the amplification by PCR and its identification. Stem-loop RT-qPCR and polyadenylation RT-qPCR are two typical approaches to this issue, and many commercial kits that use these two principles are available. However, the two-step process makes the miRNA use rate low as only a portion of the RT solution can be used for qPCR, or dilution is required to eliminate the inhibitory effect of RT solution on qPCR. Furthermore, contamination may occur during liquid transfer, leading to false positive results. In addition, real-time monitoring and thermal cycling of qPCR require precise optical components and temperature control, which limits the possibility of end-point readout and its application in point-of care testing.

Many emerging approaches have been developed to advance the miRNA detection technologies and provide alternatives to RT-qPCR.6 Isothermal reactions such as rolling circle amplification (RCA), exponential amplification reaction (EXPAR), loop-mediated isothermal amplification (LAMP), hybridization chain reaction (HCR), and catalytic hairpin assembly (CHA), etc. have been leveraged and proven their feasibility for developing miRNA assays. Despite the fact that no precise temperature control is required, the multi-step process of RCA-based methods, non-specific amplification, and high background signals of EXPAR and LAMP, and the slow kinetics and low sensitivity of HCR and CHA make those methods still time-consuming and labor-intensive and unsuitable for the detection of clinical samples. Techniques such as microfluidics, nanopore, fluorescence microscopy, nanomaterials combined with existing detection principles demonstrated advantages such as low sample volume consumption, integrity, and high sensitivity even at the single-molecule level, however, the specific apparatus or complicated manufacturing of devices dramatically increases the operational complexity. Accordingly, there is a need to address at least the aforementioned deficiencies and inadequacies.

### SUMMARY

Described herein are methods, systems, and kits for the detection of nucleic acids, for example polynucleotides such as microRNAs in a sample. Embodiments of the systems, methods, and kits of the present disclosure can detect target polynucleotides in a one-pot reaction.

The present disclosure provides methods of detecting a target polynucleotide in a sample. In embodiments, such methods include:
incubating the contents of a reaction vessel at a first isothermal temperature for a first period of time, the reaction vessel comprising: a sample comprising one or more microRNAs; a padlock probe comprising a ligation zone and a detection zone, the ligation zone comprising a polynucleotide sequence complementary to a microRNA target of interest, and the detection zone comprising a sequence capable of hybridizing with a crRNA, the ligation zone of the padlock probe comprising a nick in the sequence of the ligation zone complementary to the miRNA target of interest, wherein the nick defines the 5′ and 3′ ends of the padlock probe, and wherein the 5′ end is phosphorylated; a ribonucleoprotein complex (RNP) comprising a Cas12a CRISPR-associated (Cas) enzyme and a CRISPR-RNA (crRNA), wherein the crRNA is capable of hybridizing with the detection zone of the padlock probe; a ligase; a polymerase; and a reporter deoxyribonucleic acid (DNA) capable of producing a CRISPR-generated detectable signal or detectable molecule upon cleavage by the Cas enzyme; and
detecting the CRISPR-generated detectable signal or detectable molecule if a target microRNA is present in the sample.

The present disclosure also provides embodiments of one-pot nucleic acid detection systems for detecting a target polynucleotide in a sample. In embodiments, such systems include: a set of isothermal detection components comprising: a Cas12a CRISPR-associated (Cas) enzyme; a ligase; a polymerase; and a reporter deoxyribonucleic acid (DNA) capable of producing a CRISPR-generated detectable signal or detectable molecule upon cleavage by the Cas enzyme.

According to some embodiments, systems of the present disclosure further comprise a single reaction vessel configured to contain the elements of the system of aspect 20 in a single pot. Systems of the present disclosure can also include a crRNA configured to form a ribonucleoprotein complex (RNP) with the Cas12a enzyme, where the crRNA is provided separately from the Cas12a enzyme or wherein the crRNA is pre-combined the Cas12a enzyme to provide a RNP in the system.

In some embodiments of the systems of the present disclosure, the system further includes a padlock probe comprising a ligation zone and a detection zone, the ligation zone comprising a polynucleotide sequence complementary to the target polynucleotide, and the detection zone comprising a sequence capable of hybridizing with the crRNA, the ligation zone further comprising a nick in the sequence of the ligation zone complementary to the miRNA target of interest, wherein the nick defines the 5′ and 3′ ends of the padlock probe, and wherein the 5′ end is phosphorylated.

The present disclosure also provides shelf-stable kits for detecting a target polynucleotide in a sample. In some embodiments, kits of the present disclosure include the following components: a) a lyophilized Cas12a CRISPR-associated (Cas) enzyme and a lyophilized CRISPR-RNA (crRNA), wherein the crRNA is capable of hybridizing with the detection zone of the padlock probe, and wherein the crRNA and Cas enzyme are capable of forming a ribonucleoprotein (RNP) complex; b) a lyophilized ligase; c) a lyophilized polymerase; d) a lyophilized reporter; and instructions for combining lyophilized components a-d with a sample and a specific padlock probe, incubating the sample and the padlock probe at a first temperature for a first period of time, and detecting the detectable signal or molecule. According to some aspects of the present disclosure, the kits further include a multi-well plate, wherein each well serves as a single reaction vessel configured to contain the lyophilized components of the kit in a single pot. In some embodiment of the kits of the present disclosure, the lyophilized components are formed into a pellet and each well of the multi-well plate comprises a pellet comprising the lyophilized reaction components. According to some embodiments of the present disclosure, the kits further include a padlock probe, where the padlock probe comprises comprising a ligation zone and a detection zone, the ligation zone comprising a polynucleotide sequence complementary to the target polynucleotide, and the detection zone comprising a sequence capable of hybridizing with the crRNA, wherein the ligation zone of the padlock probe comprises a nick in the sequence of the ligation zone complementary to the miRNA target of interest, wherein the nick defines the 5′ and 3′ ends of the padlock probe, and wherein the 5′ end is phosphorylated.

According to other embodiments of the present disclosure digital, one-pot nucleic acid detection systems for detecting a target polynucleotide in a sample are also provided. Such digital, one-pot nucleic acid detection systems can include the systems or kits of the present disclosure, a single reaction vessel configured to contain the components of the system in a single pot suitable for digital detection, and a heating element to maintain the reaction vessel at a desired reaction temperature(s) for one or more set periods of time.

The present disclosure also provides point-of-care systems for visible detection of a target polynucleotide in a sample. In embodiments, the point-of-care system includes: the systems or kits of the present disclosure, where the reporter DNA comprises a polynucleotide, a first detectable molecule, and a binding moiety, wherein the detectable molecule and binding moiety are linked to opposite ends of the polynucleotide and wherein the polynucleotide is configured to be cleaved by the Cas enzyme of the RNP upon activation; and a lateral flow visible detection kit compatible with the system. In embodiments, the lateral flow visible detection kit includes a test strip comprising: a region comprising a reporter antibody specific for the first detectable molecule of the reporter DNA, the reporter antibody also comprising a second reporter molecule that produces a visible signal; a control line comprising a capture peptide capable of specifically binding the binding moiety of the reporter DNA; and a test line comprising a capture antibody specific for the first detectable molecule, wherein the capture antibody can only bind the first detectable molecule that has been cleaved from the first binding moiety.

### BRIEF DESCRIPTION OF THE DRAWINGS

Further aspects of the present disclosure will be more readily appreciated upon review of the detailed description of its various embodiments, described below, when taken in conjunction with the accompanying drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1B illustrate an embodiment of a One-pot EXTRA-CRISPR miRNA assay of the present disclosure. FIG. 1A illustrates the major components and workflow of an embodiment of an EXTRA-CRISPR assay. The padlock probe for RCA is engineered with a split ligation module for target miRNA binding, a CRISPR-Cas12a detection module whose complementary sequence activates Cas12a-crRNA complex, and a rest sequence (length and design variable). The padlock probe can have 3 different arrangements of these modules (see (i), (ii), and (iii). miRNA sequences are extracted from a sample, annealed with the padlock, and then added to a reaction tube containing the enzymes, reporter, and other reagents. The one-pot assay is carried out at 37° C. in a qPCR apparatus for real-time detection of the fluorescence signal. FIG. 1B is a schematic illustration of the mechanism of the EXTRA-CRISPR. This assay harnesses both cis- and trans-cleavage functions of the CRISPR-Cas12a system to convert linear RCA to an exponential amplification platform for miRNA detection. Briefly, the Cas12a RNP can bind and cleave the long ssDNA amplicon of RCA by its cis-activity, which generates many secondary primers containing the target sequence to initiate subsequent RCA cycles, resulting in exponential amplification of the target. Meanwhile, the amplicon-activated Cas12a RNP non-specifically cleaves the ssDNA reporters to create and amplify fluorescence signal.

FIGS. 2A-2H illustrate mechanistic studies of EXTRA-CRISPR assays of the present disclosure. FIG. 2A illustrates the effect of the padlocks without and with a PAM sequence on miR-21 detection. The assays were conducted with 1 pM miR-21 and 100 nM of each padlock probe. NC: Negative control assay with a buffer blank. FIG. 2B illustrates real-time one-pot detection of serial 10-fold dilutions of miR-21 from 1 pM to 1 nM with 1 nM Cas12a RNP. Control assays were conducted with one of three enzymes left out each time. FIG. 2C illustrates gel analysis of the products from the reactions in FIG. 2B. FIG. 2D illustrates assessment of Cas12a activities on RCA amplicons in a two-step fashion. In this case, ligation-RCA was first conducted and then the products were treated with varying amounts of RNP. M: DNA marker. FIG. 2E illustrates EXTRA-CRISPR assays using the cleaved RCA products from Reactions 2 and 3 in FIG. 2D as the targets. FIG. 2F illustrates EXTRA-CRISPR detection of the long-cut and short-cut extracts recovered from the gel bands in Lane 2 in FIG. 2D. Despite its lower quantity, the short-cut extract yielded a faster reaction kinetics than the long cut. FIG. 2G illustrates synthetic ssDNA mimicking the short-cut RCA product effectively triggers the EXTRA-CRISPR assay to produce quantitative signals. FIG. 2H illustrates exponential amplification of this synthetic short cut was observed in the one-pot assay, as opposed to linear amplification of the synthetic short cut in the two-step assay. The target concentration in both cases is 1 pM. FIG. 2I is an illustration of the length-dependent binding of the Cas12a-cleaved RCA amplicons that results in differential efficiency for the secondary ligation and RCA reactions. In contrast to the short-cut amplicon, the long cuts may hybridize with the padlock sequences in the linear forms, which terminates the ligation and exponential RCA.

FIGS. 3A-3D illustrate comparison of the kinetics and detection sensitivity of RCA, one-pot EXTRA-CRISPR, and multi-step CRISPR-assisted RCA assays. FIG. 3A illustrates an RCA-only method involving two sequential reactions of ligation and linear RCA, which can only detect 100 pM miR-21. Ligation condition: 100 nM padlock probe and 0.625 U/μL SplintR ligase. RCA condition: 2 μL ligation product, 0.2 U/μL phi29, and SYBY Green II for detection. FIG. 3B illustrates an assay that connects three tandem steps of ligation, RCA, and Cas12a readout yielded a LOD of ˜100 fM. The conditions for ligation and RCA were the same as in FIG. 3A. After RCA and denaturing of enzymes, 40 nM RNP and 0.8 μM reporter were added into the reaction. FIG. 3C illustrates an embodiment of the two-step method, where ligation and RCA were combined together, followed by fluorogenic detection using Cas12a RNP, conferring similar sensitivity with the three-step assay in FIG. 3B. Finally, FIG. 3D illustrates an embodiment of the one-pot EXTRA-CRISPR method improves the sensitivity to detect 10 fM miR-21 prior to full optimization. Assay concentrations used in FIGS. 3D and 3D: 100 nM Padlock-1, 0.625 U/μL SplintR ligase, 0.1 U/μL phi29 polymerase, 1 μM reporter, and 1 nM Cas12a RNP.

FIGS. 4A-4K illustrate optimization of embodiments of the one-pot EXTRA-CRISPR miR-21 assay. FIG. 4A illustrates that the position of the CRISPR module in a padlock sequence affects the detection signal and background level. The assays were conducted with 1 pM miR-21 and 100 nM padlock. FIG. 4B illustrates a comparison of T4 DNA ligase and SplintR ligase for the one-pot assay. The assays were conducted with 40 U/μL T4 DNA ligase or 2.5 U/μL SplintR ligase, 100 nM Padlock-1, 0.2 U/μL phi29 polymerase, 50 nM Cas12a RNP, and 1 μM reporter. The bar graphs in FIGS. 4C-4F illustrate optimization of the concentration of phi29 (FIG. 4C), padlock (FIG. 4D), Cas12a RNP (FIG. 4E), and reporter (FIG. 4F). ΔRFU, unless otherwise specified, was the signal increase from 0 min to 120 min. Error bars represent one standard deviation (S.D., n=3). The selected optimal conditions were indicated by a color background. FIG. 4G shows representative real-time curves for calibrating the one-pot assay with serial dilutions of synthetic miR-21 standards using the optimized protocol. The inset displays the curves of averaged signal for 0 (NC), 5, and 10 fM miR-21 with the shaded bands indicating one S.D. (n=5). FIG. 4H illustrates titration curves plotted at various time points for the assay calibration in FIG. 4G show a strong dependence of the assay performance on the reaction time. FIG. 4I is a diagram of the analytical figures of merit determined by the assay calibration, including LOD, sensitivity (the slope of linear calibration curve), and linear dynamic range defined by the lower limit of quantification (LLOQ) and upper limit of quantification (ULOQ). FIG. 4J illustrates linear calibration obtained with the optimal assay time of 100 min yields a LOD of 1.64 fM miR-21 calculated from 3×S.D. of the background level and a linear range from 5.47 fM to 500 fM. Error bars indicate one S.D. (n=5). FIG. 4K illustrates specificity of the EXTRA-CRISPR for detecting miR-21 against a single-mismatch miR-21 sequence (Mismatch-1) and eight different miRNAs (1 pM each). The color intensity represents the averaged signal level of two replicates.

FIGS. 5A-5G illustrate quantitative profiling of EV-derived miRNAs. FIG. 5A is a schematic illustration of an experimental procedure for comparative analysis of miRNAs in EVs isolated from cell culture media and human plasma, respectively, using both EXTRA-CRISPR and RT-qPCR. FIG. 5B illustrates calibration curves for quantifying miRNA-196a, miR-451a, and miR-1246 by EXTRA-CRISPR. Error bars: one S.D. (n=3). FIG. 5C is a graph comparing the LODs for the EXTRA-CRISPR and RT-qPCR analyses of four miRNA targets. FIG. 5D illustrates validation of specific analysis across four miRNAs (1 pM each) by the EXTRA-CRISPR. The color intensity denotes the averaged signal level of three technical replicates for each target. FIG. 5E illustrates abundance and size distribution analyses EVs isolated from serum-free media of six control and PDAC cell lines by NTA. Normal controls: Human adult dermal fibroblasts (HADF) and human primary pancreatic fibroblast (HPPF); PDAC cell lines: MIA-PaCa-2, PANC-1, PC1, and PC5. FIG. 5F shows representative TEM images illustrating PANC-1 cell-derived sEVs isolated by ultracentrifugation. Inset: spherical morphology of a sEV highlighting the lipid membrane structure. FIG. 5G is an image illustrating quality assessment of isolated sEVs with a commercial antibody array. PANC-1 cell-derived sEVs were assayed to detect eight EV-associated protein markers, including CD81, CD63, FLOT1 (Flotilin-1), ICAM1 (intercellular adhesion molecule 1), ALIX (Programmed cell death 6 interacting protein), EpCAM (Epithelial cell adhesion molecule), ANXA5 (Annexin A5), TSG101 (tumor susceptibility gene 101), and a control for cellular contamination, GM130 (cis-golgi matrix protein). FIG. 5H illustrates a comparison of the miRNA levels of MIA-PaCa-2 sEVs determined by EXTRA-CRISPR and RT-qPCR analyses of short RNA extracted from 30 μL of purified sEVs. Error bars: one S.D. (n=2 to 4 as indicated). FIG. 5I is a heatmap illustration of the normalized expression levels of miR-21, miR-196a, miR-451a, and miR-1246 in six cell line-derived sEVs measured by EXTRA-CRISPR. The miRNA expression level was normalized by the input number of sEV particles for each cell line. The color-coded miRNA level indicates the mean of two technical replicates of each assay. ND: not detected.

FIGS. 6A-6J illustrate embodiments of one-pot miRNA analysis for diagnosis of pancreatic cancer. FIG. 6A illustrates NTA of total EVs isolated by a precipitation kit from the plasma fluids of a healthy donor and a PDAC patient. FIG. 6B illustrates representative TEM images of plasma sEVs from a PDAC patient. FIG. 6C is an image illustrating a quality check of a patient-derived EV sample with the exosome antibody array. FIG. 6D illustrates a heatmap of the expression levels of individual miRNAs in the isolated plasma EVs from the PDAC patients (n=20) and healthy donors (n=15) measured by EXTRA-CRISPR. Each miRNA in each sample was tested in two technical replicates and the background-subtracted signals were adjusted by that of the positive control. The EV-Sig was defined by the weighted linear combination of four miRNAs using Lasso regression. FIG. 6E shows scatter plots illustrating individual sEV-miRNA markers and EV-Sig for discriminating the PDAC group from the control group. The middle line and error bar represent the mean and one s.e.m., respectively. P values were calculated by two-tailed Student's t-test with Welch correction. FIG. 6F illustrates ROC curves and AUC analysis of individual sEV-miRNAs and the EV-Sig for PDAC diagnosis. FIGS. 6G-6I are graphs illustrating correlation between the parallel measurements by EXTRA-CRISPR and RT-qPCR for miR-21 (FIG. 6G), miR-451a (FIG. 6H), and miR-1246 (FIG. 6I). The data points represent the mean of two replicates of each measurement by each method. Linear Deming fitting of the data points was performed to generate the linear correlation curves. (j) Comparing the EXTRA-CRISPR-based EV-Sig and the RT-qPCR tests of the four-miRNA panel for PDAC diagnosis. The RT-qPCR results of miR-21, miR-451a, and miR-1246 were assessed by the Lasso regression and ROC analysis. All statistical analyses were performed at 95% confidence level.

FIGS. 7A-7L illustrate assessment of the EXTRA-CRISPR assay for low-cost point-of-care testing. FIG. 7A illustrates an exploded view (left) and assembled view (right) of a portable smartphone-based fluorescence detector assembled with the 3D-printed body parts, a blue LED illuminator, and a consumer digital hotplate. FIG. 7B illustrates comparison of the POC device for isothermal EXTRA-CRISPR reaction to a qPCR thermal cycler (left graph, two amplification reactions with 500 fM miR-21 input were conducted by placing the tubes either on the top of the hotplate in the POC device or inside the qPCR instrument; after 2-hour reaction, the products of two reactions were all analyzed by the fluorescence detection using the smartphone-based POC device) and also real-time fluorescence detection of miR-21 using an embodiment of a portable EXTRA-CRISPR assay system of the present disclosure (right). Images were captured every two minutes using a smartphone with the exposure time of 1 s. FIGS. 7C-7E illustrate representative fluorescence and corresponding grayscale images (left) acquired for calibrating the low-cost POC system for detection of miR-21, miR-451a, and miR-1246, respectively. The grayscale images were processed to create the background-subtracted calibration curves (right) for these three miRNAs with linear least-squares regression. Error bar: one S.D. (n=2). FIG. 7F shows representative fluorescence images and FIG. 7G shows the scatter dot plots for detecting three miRNA markers in sEVs isolated from four control (H1-H4) and four PDAC (P1-P4) plasma samples using the POC device. To plot the scatter dot graph, the background-corrected mean gray values for each miRNA marker were normalized by that of 500 fM standard miRNA. FIG. 7H is a schematic illustrating the principle of a lateral flow assay for visual detection of the EXTRA-CRISPR product. FIG. 7I provides representative digital images (top) and the bar graphs for the intensity of test lines (bottom) illustrating the EXTRA-CRISPR-LFA detection of three miRNAs at variable concentrations. Error bar: one S.D. (n=3). FIG. 7J shows representative digital images, and FIG. 7K shows scatter dot plots illustrating detection of three sEV-miRNA markers in the same clinical samples as in FIG. 7F. The LFA test line signals were normalized with the averaged control line intensity measured for the negative controls, respectively. In FIG. 7G and FIG. 7K, each data point represents the mean of two technical replicates. The middle line and error bar represent the population mean and one s.e.m., respectively. P values were calculated by two-tailed Student's t-test with Welch correction. FIGS. 7L-7N illustrate a graphical comparison of the results for measuring sEV miR-451a (FIG. 7L), miR-21 (FIG. 7M), and miR-1246 (FIG. 7N) from FIGS. 7G and 7K and shows good correlation between the LFA and the smartphone POC device. Deming linear fitting was performed at the 95% confidence level.

FIGS. 8A-8B illustrate performance of variants of padlock probes with engineered secondary structure. FIG. 8A illustrates the results of one-pot reactions with each of the padlock 1 variants (padlock 1-1, 1-2, 1-3, and 1-4) with 1 pM miR-21. Tests were performed in triplicate. Error bars indicate one S.D. (n=3). FIG. 8B shows the Tm, and ΔG values of the most stable hairpin structure for each of the padlock variant DNA sequences (sequences provided in Table 2, below) as analyzed with the OligoAnalyzer (IDT).

FIG. 9 is an illustration of an embodiment of a multi-well plate EXTRA-CRISPR assay according to the present disclosure.

FIG. 10 is a graph illustrating a one-pot reaction mixing the 3-enzymes based on teachings of previous methods, showing that this method does not produce a distinguishable signal at various concentrations of target, even high amounts of target did not produce a signal distinguishable from negative control.

The figures are also further discussed in the description and examples below.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of genetics, biochemistry, molecular biology, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20-25° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

All publications and patents cited in this specification are cited to disclose and describe the methods and/or materials in connection with which the publications are cited. Publications and patents that are incorporated by reference, where noted, are incorporated by reference as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. Any terms not specifically defined within the instant application, including terms of art, are interpreted as would be understood by one of ordinary skill in the relevant art; thus, is not intended for any such terms to be defined by a lexicographical definition in any cited art, whether or not incorporated by reference herein, including but not limited to, published patents and patent applications. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

Prior to describing the various embodiments, the following definitions are provided and should be used unless otherwise indicated.

### Definitions

In describing and claiming the disclosed subject matter, the following terminology will be used in accordance with the definitions set forth below.

It must be noted that, as used in the specification and the appended claims, the singular forms “a,” “an,” and “the” include plural referents unless the context clearly dictates otherwise. Thus, for example, reference to “a cell” includes a plurality of cells. In this specification and in the claims that follow, reference will be made to a number of terms that shall be defined to have the following meanings unless a contrary intention is apparent.

As used herein, “about,” “approximately,” and the like, when used in connection with a numerical variable, generally refers to the value of the variable and to all values of the variable that are within the experimental error (e.g., within the 95% confidence interval for the mean) or within +/−10% of the indicated value, whichever is greater.

The terms “comprise”, “comprising”, “including” “containing”, “characterized by”, and grammatical equivalents thereof are used in the inclusive, open sense, meaning that additional elements may be included. It is not intended to be construed as “consists of only.”

As used herein, “consisting of” and grammatical equivalent thereof exclude any element, step or ingredient not specified in the claim.

In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, “cDNA” refers to a DNA sequence that is complementary to an RNA transcript in a cell. It is a man-made molecule. Typically, cDNA is made in vitro by an enzyme called reverse-transcriptase using RNA transcripts as templates.

As used herein with reference to the relationship between DNA, cDNA, cRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “deoxyribonucleic acid (DNA)” and “ribonucleic acid (RNA)” can generally refer to any polyribonucleotide or polydeoxyribonucleotide, which may be unmodified RNA or DNA or modified RNA or DNA. RNA can be in the form of non-coding RNA such as tRNA (transfer RNA), snRNA (small nuclear RNA), rRNA (ribosomal RNA), anti-sense RNA, RNAi (RNA interference construct), siRNA (short interfering RNA), microRNA (miRNA), or ribozymes, aptamers, guide RNA (gRNA), CRISPR RNA (crRNA), Trans-activating crRNA (tracrRNA), or coding mRNA (messenger RNA). “Polynucleotide” as used herein can generally refer to any polyribonucleotide or polydeoxyribonucleotide.

As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA.

As used herein, the term “exogenous DNA” or “exogenous nucleic acid sequence” or “exogenous polynucleotide” refers to a nucleic acid sequence that was introduced into a cell, organism, or organelle via transfection. Exogenous nucleic acids originate from an external source, for instance, the exogenous nucleic acid may be from another cell or organism and/or it may be synthetic and/or recombinant. While an exogenous nucleic acid sometimes originates from a different organism or species, it may also originate from the same species (e.g., an extra copy or recombinant form of a nucleic acid that is introduced into a cell or organism in addition to or as a replacement for the naturally occurring nucleic acid). Typically, the introduced exogenous sequence is a recombinant sequence.

As used herein, the terms “complementary to” can refer to any polynucleotide sequence having sufficient complementarity with a target polynucleotide sequence to hybridize with the target sequence or sequence-specific binding of a CRISPR complex to the target sequence. The degree of complementarity between a polynucleotide complementary to its target sequence (Cas12a sequence or miRNA of interest) and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g. the Burrows Wheeler Aligner), ClustalW, Clustal X, BLAST, Novoalign (Novocraft Technologies, ELAND (Illumina, San Diego, Calif.), SOAP (available at soap.genomics.org.cn), and Maq (available at maq.sourceforge.net). A polynucleotide sequence complementary to a target sequence can be about or more than about 5, 10, 11, 12, 13, 14, 15, 16, 17, 18, 19, 20, 21, 22, 23, 24, 25, 26, 27, 28, 29, 30, 35, 40, 45, 50, 75, 90, 100, 110, 112, 115, 120, 130, 140, or more nucleotides in length. The polynucleotide sequence complementary to a target sequence can include a nucleotide sequence that is complementary to a target DNA sequence. This portion of the guide sequence can be referred to as the complementary region of the target nucleotide (such as a miRNA) or the CRISPR RNA (crRNA). Another portion of the guide sequence serves as a binding scaffold for the CRISPR-associated (Cas) nuclease.

A guide polynucleotide can be less than about 150, 125, 75, 50, 45, 40, 35, 30, 25, 20, 15, 12, or fewer nucleotides in length. The ability of a guide polynucleotide to direct sequence-specific binding of a CRISPR complex to a target sequence may be assessed by any suitable assay. For example, the components of a CRISPR system sufficient to form a CRISPR complex, including the guide polynucleotide to be tested, may be provided to a host cell having the corresponding target sequence, such as by transfection with vectors encoding the components of the CRISPR sequence, followed by an assessment of preferential cleavage within the target sequence. Similarly, cleavage of a target polynucleotide sequence may be evaluated in a test tube by providing the target sequence, components of a CRISPR complex, including the guide polynucleotide to be tested and a control guide polynucleotide different from the test guide polynucleotide, and comparing binding or rate of cleavage at the target sequence between the test and control guide polynucleotide reactions. Other assays are possible, and will occur to those skilled in the art.

As used herein, “nucleic acid,” “nucleotide sequence,” and “polynucleotide” can be used interchangeably herein and can generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

As used herein, “isolated” means separated from constituents, cellular and otherwise, in which the polynucleotide, peptide, polypeptide, protein, antibody, or fragments thereof, are normally associated with in nature. A non-naturally occurring polynucleotide, peptide, polypeptide, protein, antibody, or fragments thereof, do not require “isolation” to distinguish it from its naturally occurring counterpart.

As used herein, the terms “optional” or “optionally” means that the subsequently described event or circumstance can or cannot occur, and that the description includes instances where said event or circumstance occurs and instances where it does not.

As used herein, the term “specific binding” or “preferential binding” can refer to non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. Binding of two or more entities may be considered specific if the equilibrium dissociation constant, K_{d}, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a K_{d} of greater than 10⁻³ M). In some embodiments, specific binding, which can be referred to as “molecular recognition,” is a saturable binding interaction between two entities that is dependent on complementary orientation of functional groups on each entity. Examples of specific binding interactions include primer-polynucleotide interaction, aptamer-aptamer target interactions, antibody-antigen interactions, avidin-biotin interactions, ligand-receptor interactions, metal-chelate interactions, hybridization between complementary nucleic acids, etc.

As used herein, the term “recombinant” or “engineered” can generally refer to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc. Recombinant or engineered can also refer to the polypeptide encoded by the recombinant nucleic acid. Non-naturally occurring nucleic acids or polypeptides include nucleic acids and polypeptides modified by man.

As used herein, the term “vector” or is used in reference to a vehicle used to introduce an exogenous nucleic acid sequence into a cell. A vector may include a DNA molecule, linear or circular (e.g. plasmids), which includes a segment encoding a polypeptide of interest operatively linked to additional segments that provide for its transcription and translation upon introduction into a host cell or host cell organelles. Such additional segments may include promoter and terminator sequences, and may also include one or more origins of replication, one or more selectable markers, an enhancer, a polyadenylation signal, etc. Expression vectors are generally derived from yeast or bacterial genomic or plasmid DNA, or viral DNA, or may contain elements of both. In some embodiments of the present disclosure viral vectors are used, such as single stranded viral vectors, such as adeno-associated virus (AAV) vectors.

As used herein, “operatively linked” in the context of recombinant DNA molecules, vectors, and the like refers to the regulatory and other sequences useful for expression, stabilization, replication, and the like of the coding and transcribed non-coding sequences of a nucleic acid that are placed in the nucleic acid molecule in the appropriate positions relative to the coding sequence so as to effect expression or other characteristic of the coding sequence or transcribed non-coding sequence. This same term can be applied to the arrangement of coding sequences, non-coding and/or transcription control elements (e.g., promoters, enhancers, and termination elements), and/or selectable markers in an expression vector. “Operatively linked” can also refer to an indirect attachment (i.e., not a direct fusion) of two or more polynucleotide sequences or polypeptides to each other via a linking molecule (also referred to herein as a linker).

As used herein, “transforming” when used in the context of engineering or modifying a cell, refers to the introduction by any suitable technique and/or the transient or stable incorporation and/or expression of an exogenous gene in a cell.

As used herein, “selectable marker” refers to a gene whose expression allows one to identify cells that have been transformed or transfected with a vector containing the marker gene. For instance, a recombinant nucleic acid may include a selectable marker operatively linked to a gene or insert of interest and a promoter, such that expression of the selectable marker indicates the successful transformation of the cell with the gene or insert of interest. Examples of selectable markers include, but are not limited to, DNA and/or RNA segments that contain restriction enzyme sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); the generation of new primer sites for PCR (e.g., the juxtaposition of two DNA sequence not previously juxtaposed), the inclusion of DNA sequences not acted upon or acted upon by a restriction endonuclease or other DNA modifying enzyme, chemical, etc.; epitope tags (e.g. FLAG- and His-tags), and, the inclusion of a DNA sequences required for a specific modification (e.g., methylation) that allows its identification. Other suitable markers will be appreciated by those of skill in the art.

As used herein, “promoter” includes all sequences capable of driving transcription of a coding or a non-coding sequence. In particular, the term “promoter” as used herein refers to a DNA sequence generally described as the 5′ regulator region of a gene, located proximal to the start codon. The transcription of an adjacent coding sequence(s) is initiated at the promoter region. The term “promoter” also includes fragments of a promoter that are functional in initiating transcription of the gene.

As used herein, “organism”, “host”, and “subject” refers to any living entity comprised of at least one cell. A living organism can be as simple as, for example, a single isolated eukaryotic cell or cultured cell or cell line, or as complex as a mammal, including a human being, and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans).

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents and are meant to include future updates.

Reference throughout this specification to “one embodiment”, “an embodiment”, “another embodiment”, “some embodiment,” means that a particular feature, structure or characteristic described in connection with the embodiment is included in at least one embodiment of the present invention. Thus, appearances of the phrases “in one embodiment,” “in an embodiment,” “in another embodiment”, or “in some embodiment” in various places throughout this specification are not necessarily all referring to the same embodiment, but they may. Furthermore, the particular features, structures or characteristics may be combined in any suitable manner, as would be apparent to a person skilled in the art from this disclosure, in one or more embodiments. Furthermore, while some embodiments described herein include some, but not other, features included in other embodiments, combinations of features of different embodiments are meant to be within the scope of the invention. For example, in the appended claims, any of the claimed embodiments can be used in any combination.

### DISCUSSION

In accordance with the purpose(s) of the present disclosure, as embodied and broadly described herein, embodiments of the present disclosure, in some aspects, relate to methods, systems, and kits for detecting a target polynucleotide. According to some embodiments, the methods, systems, and kits for detecting a target polynucleotide harness aspects of CRISPR cis and trans cleavage as well as rolling circle amplification to provide a simple, one-pot detection reaction. In certain aspects, a target nucleotide is a microRNA (miRNA). In other aspects, the target nucleotide is a biomarker (miRNA or other polynucleotide biomarker) of an illness, such as cancer. In some embodiments, the target nucleotide is a miRNO that is a biomarker for pancreatic ductal adenocarcinoma (PDAC). Additional aspects of the current disclosure are provided in the discussion below.

### Overview

As described in greater detail in the examples below, the methods, systems, and kits of the present disclosure combine the use of Cas12a enzymes, a padlock probe, and other reaction enzymes (e.g., ligase, polymerase, etc.) for detection of nucleic acid targets, such as microRNAs, in a single pot reaction. In some aspects of the present disclosure the methods, systems, and kits of the present disclosure combine rolling circle amplification and CIRSPR-mediated polynucleotide capture and detection. In other aspects, the nucleic acid detection can be isothermal (e.g., PCR).

### CRISPR-Associated Enzyme

CRISPR-associated (Cas) enzymes (also known as CRISPR effector protein) are enzymes which can bind to a guide RNA (crRNA or sgRNA) to form a ribonucleoprotein complex (RNP) and to a complementary target polynucleotide sequence, forming a CRISPR/Cas complex, and can cleave the target sequence (cis cleavage). Some Cas enzymes possess both cis- and trans-cleavage activity, where trans-cleavage activity is activated upon binding of the CRISPR/Cas complex with the target sequence. Activation of the trans cleavage activity allows cleavage of probes also included in the reaction mixture, such that the probes produce a detectable signal or molecule that indicates the presence of the target sequence.

In embodiments of the present disclosure, the Cas enzymes are Cas12a enzymes. Cas12a enzyme have been studied and harnessed for nucleic acid detection due to their robust cis and trans cleavage activity. In some embodiments, the Cas12a enzymes are from a digestive-tract bacterium Lachnospiraceae. In an embodiment, the enzyme can be LbCas12a (e.g., Lba Cas12a), such as those described in Chen et al., CRISPR-Cas12a target binding unleashes indiscriminate single-stranded DNase activity. SCIENCE, 15 Feb. 2018; Vol. 360; Issue 6387; pp. 436-439 DOI: 10.1126/science.aar6245, which is incorporated by reference as if fully set forth herein, in particular relating to LbCas12a enzymes.

crRNA

In embodiments, the methods, systems, and kits of the present disclosure include a crRNA to couple with the Cas enzyme to form a CRISPR/Cas complex, also referred to herein as a ribonucleoprotein complex or RNP. The crRNAs of embodiments of the present disclosure have a portion of a sequence that is complementary to the Cas enzyme (e.g., a Cas12a enzyme such as IbCas12a) so that it can form a complex with the Cas. Another portion of the crRNA is complementary to a sequence amplified in the systems of the present disclosure upon presence of a target sequence, such that the RNP (Cas and crRNA complex) can bind the amplified sequence and detect the presence of the target, as explained in greater detail below.

In some embodiments, a crRNA is about 130 base pairs. In embodiments, the length of the guide sequence of the crRNA is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, the guide sequence is less than about 75, 50, 45, 40, 35, 30, 25, 20, 15, 12, or fewer nucleotides in length. In one embodiment, the guide sequence is 10-30 nucleotides long. In embodiments, the crRNA is configured to interact with or has sequences complementary to sequences or conserved sequences of Cas12a enzymes.

In embodiments, the CrRNA binds the amplified target polynucleotides and forms a CRISPR/Cas complex with the CAS12a to activate the trans cleavage activity of the Cas12, which cleaves probes in the reaction vessel to create a detectable signal and indicate the presence of the target polynucleotide in the sample, thereby providing information that can be utilized for the diagnosis of the specific condition/disease associated with the target polynucleotide.

In embodiments, the target polynucleotide in the sample can be amplified by the isothermal amplification components described below, and then the CrRNA binds the amplified target polynucleotides and forms a CRISPR/Cas complex with the Cas12a to activate the trans cleavage activity of the Cas12a, which cleaves probes in the reaction vessel to create a detectable signal and indicate the presence of the target polynucleotide in the sample, thereby diagnosing the specific condition/disease associated with the target polynucleotide.

In embodiments of the present disclosure, the crRNA can comprise the sequence UAAUUUCUACUAAGUGUAGAUCGUCGCCGUCCAGCUCGACC (SEQ ID NO: 24), where nucleotides 1-21 represent the conserved sequence for Lba cas12a, and nucleotides 22-41 represent the variable sequence. If the variable sequence changes, the detection sequences can be changed correspondingly. While the description herein and the examples below describe the methods, systems, and kits with respect to the above crRNA (for forming a complex with LbaCas12a) the system could be modified by methods known to those of skill in the art to provide other crRNAs complementary to other Cas enzymes.

In the present disclosure, a complex formed by the Cas enzyme (e.g., Cas12a) and the crRNA, the RNP can be provided by supplying the crRNA and Cas enzymes separately to the systems/kits where they can form a complex upon combination with a sample. In this way the crRNA's can be changed and/or customized if desired. However, since the crRNA for the methods, kits, and systems of the present disclosure does not need to be customized to the target sequence, the crRNA does not need to be changed depending on the target. Thus, in other embodiments, the RNP complex can be pre-formed in the system (e.g., by pre-combining and/or incubating the crRNA and Cas enzyme) prior to addition to a reaction vessel. The Cas enzymes, crRNAs, and RNP complexes can be lyophilized for systems and kits of the present disclosure for stability and storage.

### Padlock Probe

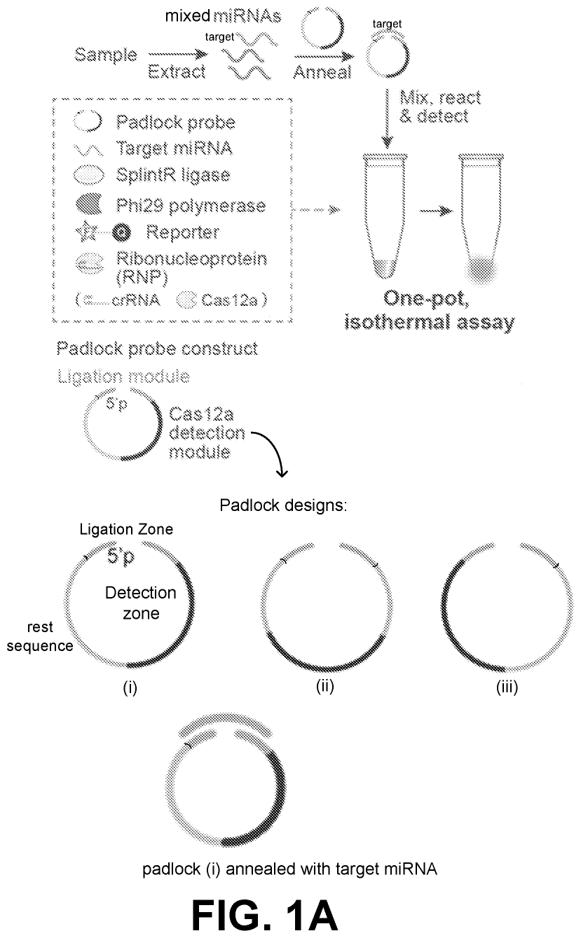

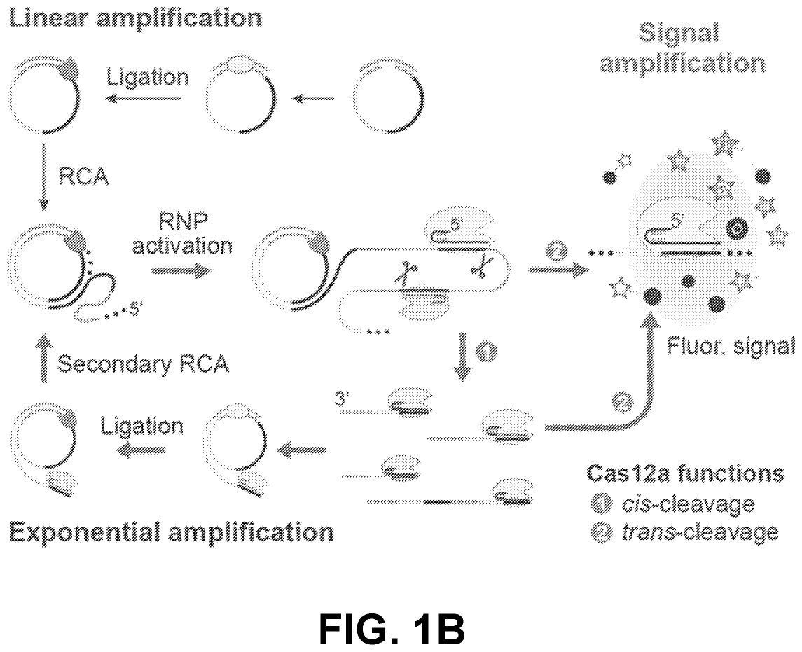

The padlock probe of the present disclosure provides the elements for recognition/capture of a target sequence as well as amplification to produce an amplification sequence that is then recognized by the RNP (CrRNA/Cas complex). When the padlock probe couples with the target sequence, this allows ligation of the padlock probe, which activates a cascade of events including amplification of the padlock sequence, activation of the RNP, which induces trans cleavage activity to cleave a probe to produce a detectable signal as well as cis cleavage of the amplification product to produce additional sequences to unlock other padlock probes to induce exponential amplification and signal as illustrated in FIGS. 1A-1B and described in Examples 1 and 2 below.

To this end, the padlock contains various zones or modules with different purposes/functions. Referring to FIG. 1A(i), the padlock contains a ligation zone (medium gray), a detection zone (black) and the rest sequences (light gray). The ligation zone can be used for the capture of target miRNA (which then allows ligation of the padlock probe and initiation of polymerization). The detection zone can be used for the activation of Cas/crRNA complexes (RNP), and the rest sequence can be used for adjusting the length of the padlock probe to minimize the rigidity of the circular padlock and to adjust the secondary structure of the padlock probe. The padlock can have various configurations, depending on the location of the detection zone and rest sequences, as depicted in FIG. 1A (i), (ii), and (iii).

The ligation zone (FIG. 1A, medium gray) is always located at the 5′-end and 3′-end of the padlock probe, and the 5′-end is phosphorylated. The ligation zone is the sequence that is complementary to the target miRNA (or target polynucleotide as used herein), and it comprises a nick within the sequence to define the 5′ and 3′ ends of the padlock probe. The 5′ end of the padlock probe is phosphorylated. The nick in the padlock can be located at any position of the miRNA as long as the hybridization is stable (Tm>37° C.). Since the sequence of the ligation zone is complementary to the target miRNA, it can anneal with the target miRNA as shown in FIG. 1A. Annealing of the target miRNA allows ligase to seal the nick and the polymerase to induce rolling circle amplification of the padlock sequence, including the detection zone.

The detection zone (FIG. 1A, black) can be located at any position (in the right (i), middle (ii), or left (iii)). In the embodiment illustrated in FIG. 1A (i) and (iii), the detection zone is flanked one side by one of the parts of the ligation sequences and on the other side by the rest sequence, and in the embodiment illustrated in FIG. 1A(iii) the detection zone is flanked on both sides by the rest sequence and then the ligation sequences. In all configurations, the 5′ and 3′ ends of the padlock probe are defined by the ligation zone, not the detection zone or rest sequences. The detection zone is the sequence whose complementary sequences can be hybridized with crRNA of cas12a. Since the amplification product of the padlock sequence is a ssDNA, no PAM sequence (TTTV, V=A, G, C) is needed in the 5′ end of the detection zone. Thus, in embodiments of the present disclosure, the padlock probe does not contain a PAM sequence. The configuration and mechanism of the padlock probe are described in greater detail in Examples 1 and 2 below.

The total length of the padlock probe can be from about 40-100 nt, and secondary structures can be minimized/eliminated by adjusting the rest (light gray) sequence (FIG. 1A), as discussed in greater detail in Examples 1 and 2, below. In embodiments padlock probe can have a sequence of SEQ ID NOs: 1-4, although the ligation sequence portion can be changed depending on the target.

In embodiments, the target polynucleotide can be a microRNA associated with a specific condition/disease, such as cancer, genetic disease, or infectious agent (e.g., bacteria, virus, fungal, etc.). The target miRNA can bind to the ligation zone, whose design is complementary to a target of interest (FIG. 1B), and can bridge the “nick” in the ligation zone upon binding to the padlock probe. Thus, the ligation sequence of the padlock probe is customized depending on the target to be detected. In embodiments, the target polynucleotide is an miRNA that is a biomarker for a condition, and thus the padlock probe is designed such that the ligation sequence is complementary to the sequence of the miRNA biomarker. While the embodiments discussed in detail herein are directed to detection of miRNA, the padlock probes can be designed to detect various polynucleotide sequences in addition to miRNAs.

### Reporter DNA

As used herein, the reporter refers to a polynucleotide-based molecule that can be cleaved by an activated CRISPR-associated (Cas) enzyme with a trans-cleavage activity to produce a detectable signal or a detectable molecule. The probe comprises an oligonucleotide element. The oligonucleotide element contains a sequence cleavable by the activated Cas enzyme. In one embodiment, a first end of the oligonucleotide element in the probe is linked to a fluorophore; and a second end of the oligonucleotide element in the probe is linked to a quencher of the fluorophore. In some embodiments of the present disclosure, the probes are configured to be cleaved by a Cas12a enzyme (in an activated crRNA/Cas complex), such that the detectable signal or molecule can be produced upon binding of the crRNA/CAs12a complex to the target polynucleotide.

The DNA reporter can be a ssDNA modified with a fluorophore and a quencher (Q). To keep the FRET phenomenon, the length of the reporter should better not exceed 30 nt. TA-rich sequences are preferred due to the preference of Cas12a for TA-rich sequences.

A detectable molecule may be any molecule that can be detected by methods known in the art. In one embodiment, the detectable molecule is one member of a binding pair and can be detected by binding to another member of the binding pair. Examples of binding pairs include, but are not limited to, antibody-antigen pairs, enzyme-substrate pairs, receptor-ligand pairs, and streptavidin-biotin.

In one embodiment, the oligonucleotide element in the probe is ssDNA, since Cas12 trans cleavage can cut DNA. Since Cas12 enzymes preferentially cleaves DNA with an A/T rich sequence, in embodiments the oligonucleotide element of the probe includes at least 55%, 60%, 65%, 70%, 75%, 80%, 90%, or 95% of A and/or T. In embodiments the oligonucleotide element of the probe is a ssDNA and is about 80% of A and/or T. In some embodiments the oligonucleotide element is TA-rich or TA-only, and is about 2-10 nucleotides. In one embodiment, the ssDNA consists of A and/or T. In one embodiment, the oligonucleotide element is TTATT. In some embodiments, the oligonucleotide element is primarily or only T “poly T”, and in some embodiments it is polyT and about 2-10 nucleotides in length. In embodiments it is an “8-mer poly T” (TTTTTTTT).

The detectable molecule can comprise one or more of FAM, FITC, Cy3, Cy5, HEX, and TAMRA. The quencher can be various quenchers know to those of skill in the art, such as IABkFQ.

In an embodiment, the reporter DNA is FAM-TTATT-Q (e.g., 5-FAM-TTATT-IABkFQ-3′). In one embodiment, the reporter DNA can be, but is not limited to a HEX-FQ reporter, such as HEX-TTATT-FQ. In another embodiment, the reporter DNA comprises HEX-polyT-Quencher (HEX-FQ). In embodiments, the reporter DNA can also be and FITC probe or a Cy5 probe, such as, but not limited to FITC-polyT-Quencher and Cy5-PolyT-quencher. In embodiments, the probe produces a fluorescent signal that can be detected visually or by a digital device, such as a smartphone, as described in greater detail in Example 1 and accompanying figures.

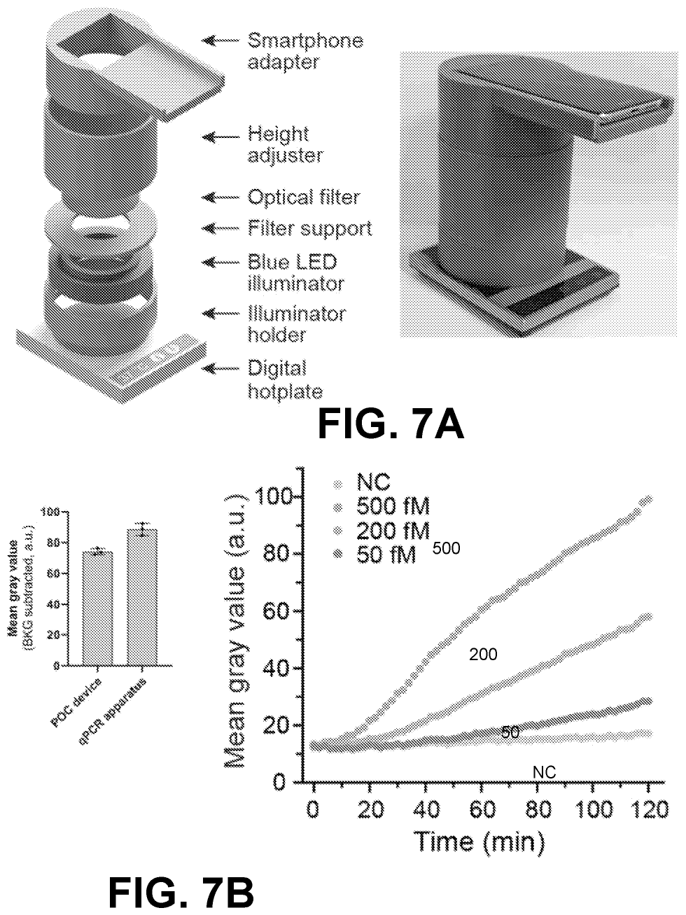

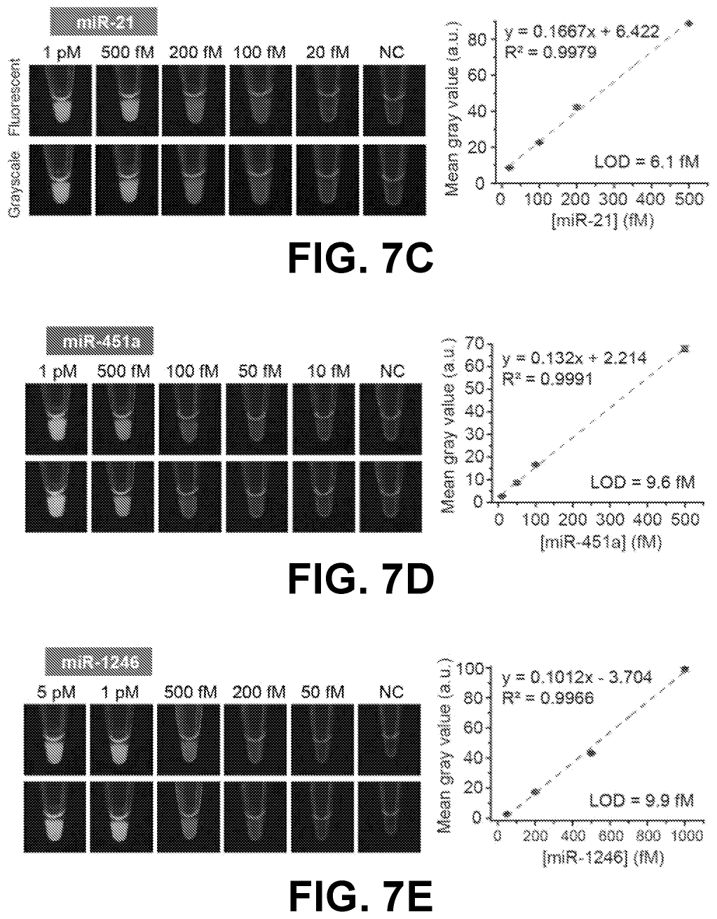

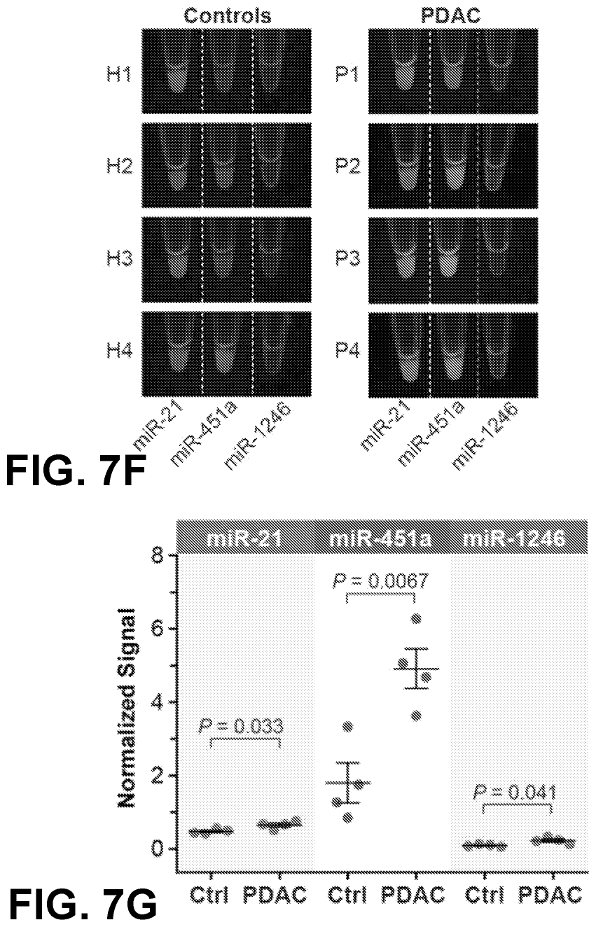

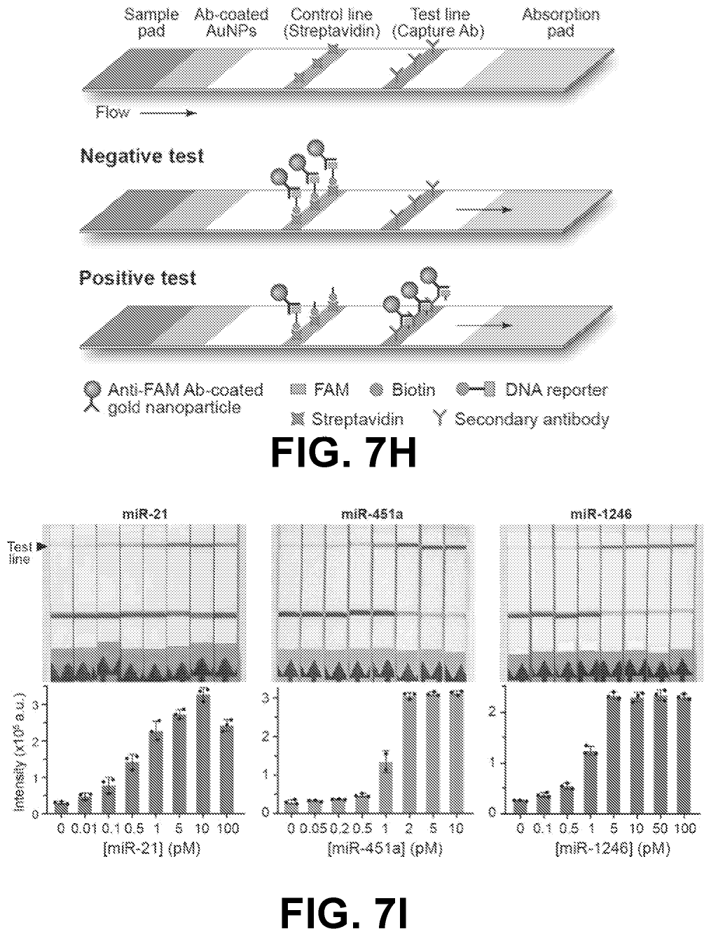

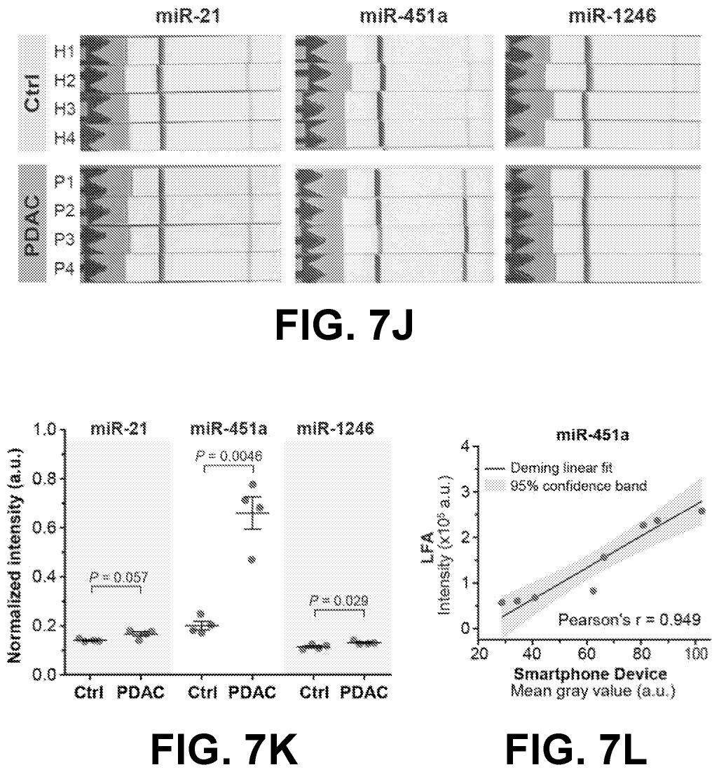

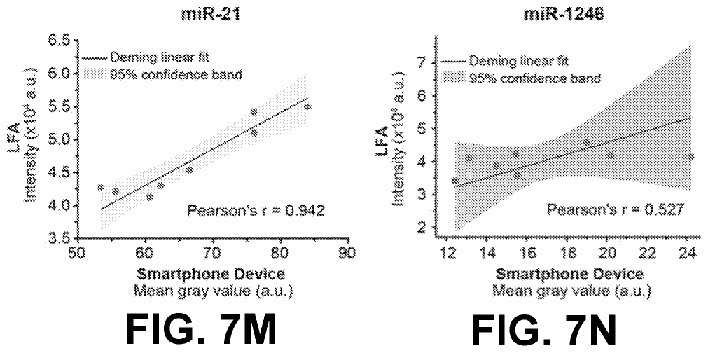

In embodiments of the disclosure adapted for a lateral flow assay for visual detection, additional probes can be provided in a lateral flow device that can bind to cleaved primary probes on a test strip in order to produce a positive signal. Such embodiments are described in greater detail in Example 1 below and FIGS. 7H-7J.

### Methods

The present disclosure also includes one-pot methods of detecting a target polynucleotide in a sample. In embodiments, the methods can include detecting a target miRNA in a sample.

Methods of detecting a target polynucleotide in a sample can comprise incubating the contents of a reaction vessel at a first isothermal temperature for a first period of time, the reaction vessel comprising: a sample comprising one or more microRNAs, a padlock probe; a Cas12a CRISPR-associated (Cas) enzyme-CRISPR-RNA (crRNA) complex (ribonucleoprotein, RNP); a ligase; a polymerase; and a reporter deoxyribonucleic acid (DNA); detecting the CRISPR-generated detectable signal or detectable molecule if the target polynucleotide is present in the sample. The reaction vessel can also include additional reaction components such as buffers, water, and other elements described in greater detail below. In some embodiments, all of the reaction components can be added to a reaction vessel at the same time, and in other embodiments, some of the components are added first and various incubation steps may be taken and other reaction components added at different intervals. Some of these embodiments are described below.

In an embodiment, the first incubation time can be varied from 10 min to 3 h or a longer time depending on the target concentration, if the target concentration is high, such as above 10 pM, a good signal intensity at 10-20 min can be obtained. If the target concentration is low, incubation time can be increased as needed.

Any one or more of padlock probes, reporter DNAs, cas12a enzymes, crRNAs, ligases, and/or polymerases as described herein can be employed according to methods as described herein. Methods as described herein can further comprise providing the sample. Methods as described herein can further comprise placing the sample in a single reaction vessel. Methods as described herein can further comprise providing the sample in a single reaction vessel prior to incubating the vessel at a first temperature. In certain aspects, the first isothermal temp is about 16 to about 48° C. In certain aspects the first isothermal temp is about 37° C.

In certain aspects, methods as described herein further comprise pre-incubating the sample comprising one or more microRNAs and the padlock probe at a second temperature for a second period of time before incubating at the first temperature and first period of time in the reaction vessel. The second temperature can be about 45 to about 99° C. The second temperature can be about 80° C. The second period of time can be about 1 to about 30 minutes. The second period of time can be about 5 minutes.

Methods as described herein can be used on a sample from a subject having or suspected of having cancer, and the one or more microRNAs to be detected in the sample can be one or more biomarkers of the cancer. The padlock probe can be designed such that the ligation zone is complementary to the sequence of the miRNA that is the biomarker for the cancer, such that the padlock probe binds the miRNA biomarker for the cancer and the assay amplifies and produces a detectible signal if the miRNA biomarker is present in a patient sample. IN embodiments, the miRNA is a biomarker for a pancreatic ductal adenocarcinoma (PDAC). The one or more microRNAs to be detected in a sample can be one or more biomarkers of PDAC, such as, but not limited to miR-21, miR-196a, miR-451a, and miR-1246, (SEQ ID NOs: 14, 20, 22, and 23, respectively), and the like (e.g., SEQ ID NOs: 14-23, as described in greater detail in the Examples below).

In methods as described herein, RNP is formed by incubating the cas12a enzyme and crRNA at a third temperature for a third period of time before placing in the reaction vessel. The third temperature can be about 4 to about 48° C. The third temperature can be about 37° C. The third period of time can be about 10 to about 180 minutes. The third period of time can be about 30 mins.

In embodiments of the systems, kits, and methods of the present disclosure the concentration of the RNP is optimized so that it is present in a sufficient concentration to produce a signal but not so much that the endonuclease activity (cis and trans) of the RNP complex when hybridized to target padlock sequence does not begin to cleave all of the ssDNA components of the system and produce no signal. In embodiments the concentration of RNP in the system is less than 50 nM; in embodiments it is less than about 25 nM; in embodiments it is about 10 nM or less; in embodiments it is from about 0.1-10 nM; in embodiments it is about 0.5-5 nM; and in other embodiments it is about 0.5 nM.

Methods as described herein can further be run on digital polymerase chain reaction (dPCR) systems, such as the QuantStudio Absolute Q Digital PCR System from ThermoFisher Scientific and the droplet digital PCR (ddPCR) systems from Bio-Rad.

Additional description of embodiments of methods of the present disclosure are provided in the examples below. Other methods for use of the isothermal amplification/CRISPR/Cas systems of the present disclosure can be appraised by one of skill in the art.

### Systems

Described herein are systems for detecting a target polynucleotide using the padlock probes, Cas enzymes, crRNAs, reporters, and isothermal reaction components (e.g, ligase, polymerase, etc.) of the present disclosure described above. In certain aspects as described herein, a system can be a one-pot nucleic acid detection system for detecting a target polynucleotide in a sample. The system can include a set of isothermal detection components comprising: a Cas12a CRISPR-associated (Cas) enzyme; a ligase; and a polymerase.

Systems as described herein can further comprise a single reaction vessel configured to contain the elements of systems, methods, and kits as described herein in a single pot. Systems as described herein can further comprise a heating element to maintain the reaction vessel at a temperature of about 60-90° C. and 25-40° C. In embodiments the heating element is a small portable heating element, such as a digital hot plate. Embodiments of systems of the present disclosure can also include a device/support structure adapted to combine the reaction vessel, heating element, and a personal computing device for detecting and/or analyzing signal produced by the system. For instance, a device can be adapted to hold the reaction vessel in proximity with a heating element adapted to maintain the temperature of the reaction vessel and one or more set temperatures for one or more set times. The device can also be adapted to hold a personal computing device (e.g., a smartphone, smart tablet, laptop, etc.) in visual detection range of the reaction vessel. Thus, in embodiments the systems of the present disclosure include and are compatible with a handheld, portable digital device, such as a smartphone. Embodiments of such a system are described in Example 1 below and illustrated in FIGS. 7A-7G.

Systems as described herein can further comprise one or more of (individually or in combination) a crRNA, a padlock probe, or a reporter DNA as described herein. The reporter DNA can be a single-stranded reporter DNA and comprise a detectable molecule.

Systems as described herein can further comprise one or more of deoxyribonucleoside triphosphates (dNTPs), bovine serum albumin (BSA), a reaction buffer, and/or water.

dNTPs can comprise dATP, dTTP, dCTP, dGTP mixed with equal molar ratio. The dNTP concentration according to systems, kits, and methods as described herein can be about 50 uM-5 mM. In an embodiment, the dNTP concentration can be about 400 uM.

The BSA concentration according to methods, systems, and kits as described herein can be about 0.02 mg/mL-2 mg/mL. In embodiments, the concentration can be about 0.2 mg/mL.

The reaction buffer according to methods, systems, and kits as described herein can comprise Tris-HCl (at a concentration of about 20-150 mM); MgCl (at a concentration of about 5-50 mM); ATP (at a concentration of about 0.5-5 mM); DTT (at a concentration of about 5-50 mM); and have a pH of about 7.2-7.9 (at 25° C.). In an embodiment, the reaction buffer can comprise the following: 50 mM Tris-HCl; 10 mM MgCl2; 1 mM ATP; 10 mM DTT; and have a pH of about 7.5 at 25° C.

Systems as described herein can comprise one or more of a padlock probe, a cas12a enzyme, a crRNA, a ligase, and/or a polymerase as described herein. Systems of the present disclosure can also include padlock probes specific for different target polynucleotides to provide for simultaneous detection of more than one target. Alternatively, systems can include multi-well plate arrays where different wells contain different padlock probes for detecting different taret polynucleotides in different wells. Each well can have the same sample or multiple samples can be tested at the same time in different wells of the array. Various configurations can be envisioned and some are disclosed in greater detail below.

Systems as described herein can further comprise one or more components for digital PCR (dPCR), for example reagents, consumables, and/or multi-well array plates from ThermoFisher compatible with systems such as the QuantStudio Absolute Q Digital PCR System and the ddPCR systems from Bio-Rad. Systems as described herein can otherwise be configured for use with digital PCR systems such as, but not limited to, the aforementioned systems.

One or more of the above-described reaction components of the present disclosure can be lyophilized to increase shelf stability, transportability, point-of-care use of the systems of the present disclosure.

The systems of the present disclosure can also be adapted for use with lateral flow devices to allow for visual point-of-care testing without the need for any specialized equipment for detection. Embodiments of such systems are described in greater detail below and illustrated in FIGS. 7H-7N.

### Kits

The present disclosure also includes kits including the system components described above to be combined with a sample and instructions for use. In embodiments, the kit is shelf-stable, such that it can be transported and stored at ambient temperatures until use. In embodiments, the contents of the kit are lyophilized, such that they are non-reactive and stable until combined with a sample and incubated at elevated temperatures for isothermal amplification and CRISPR/Cas detection.

In embodiments, kits of the present disclosure shelf-stable kit for detecting a target polynucleotide in a sample in a single-pot including one or more (in any combination) of the following components: a) a lyophilized Cas12a CRISPR-associated (Cas) enzyme and a lyophilized CRISPR-RNA (crRNA), wherein the crRNA is capable of hybridizing with the detection zone of the padlock probe, and wherein the crRNA and Cas enzyme are capable of forming a ribonucleoprotein (RNP) complex (although in embodiments, the crRNA can be provided separately from the Cas enzyme, e.g., separately from the kit); b) a lyophilized ligase; c) a lyophilized polymerase; d) a lyophilized reporter; and instructions for combining components a-c with a sample, incubating the sample at a first temperature for a first period of time, and detecting the detectable signal or molecule. The instructions can be according to any of the methods as described herein.

The cas12a enzyme can be a Lba Cas12a (cpf1). The ligase can be a SplintR ligase or a T4 DNA ligase, sequences and examples of which are known in the art. In an embodiment, the ligase is a SplintR ligase. The polymerase can be one or more of a Phi29 polymerase, Bst polymerase, Klenow Fragment. In embodiment, the polymerase is a phi29 polymerase. The kit further can comprise additional reagents, components, and consumables for the amplification and detection of nucleic acids (such as miRNAs). Such components can include one of more of lyophilized isothermal amplification buffer, deoxyribonucleoside triphosphates (dNTPs), bovine serum albumin (BSA), primers, and/or water (such as those described in systems above).

Kits as described herein can further comprise one or more padlock probes as described herein. Kits as described herein can further comprise one or more crRNAs as described herein. Kits as described herein can further comprise one or more reporter DNAs as described herein. Kits as described herein can further comprise one or more components for digital PCR (dPCR), for example reagents, consumables, and/or multi-well array plates from ThermoFisher compatible with systems such as the QuantStudio Absolute Q Digital PCR System and the ddPCR systems from Bio-Rad. Kits as described herein can be otherwise configured for use in such systems, such as described above.

Kits of the present disclosure can also be used in combination with the systems above, such as those including a heating element and/or a handheld portable computing device (e.g., smartphone), such as described above.

Additional details regarding the methods, systems, and kits of the present disclosure are provided in the Examples below. The specific examples below are to be construed as merely illustrative, and not limitative of the remainder of the disclosure in any way whatsoever. Without further elaboration, it is believed that one skilled in the art can, based on the description herein, utilize the present disclosure to its fullest extent.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and protected by the following claims.

### Various Aspects and Embodiments of the Present Disclosure

The present disclosure further includes the following aspects and embodiments.

Aspect 1: A method of detecting a target polynucleotide in a sample, the method comprising:
incubating the contents of a reaction vessel at a first isothermal temperature for a first period of time, the reaction vessel comprising: a sample comprising one or more microRNAs; a padlock probe comprising a ligation zone and a detection zone, the ligation zone comprising a polynucleotide sequence complementary to a microRNA target of interest, and the detection zone comprising a sequence capable of hybridizing with a crRNA, the ligation zone of the padlock probe comprising a nick in the sequence of the ligation zone complementary to the miRNA target of interest, wherein the nick defines the 5′ and 3′ ends of the padlock probe, and wherein the 5′ end is phosphorylated; a ribonucleoprotein complex (RNP) comprising a Cas12a CRISPR-associated (Cas) enzyme and a CRISPR-RNA (crRNA), wherein the crRNA is capable of hybridizing with the detection zone of the padlock probe; a ligase; a polymerase; and a reporter deoxyribonucleic acid (DNA) capable of producing a CRISPR-generated detectable signal or detectable molecule upon cleavage by the Cas enzyme; and
detecting the CRISPR-generated detectable signal or detectable molecule if a target microRNA is present in the sample.

Aspect 2: The method of aspect 1, further comprising providing the sample, padlock probe, RNP, ligase, polymerase, and reporter DNA in a single reaction vessel prior to incubating the vessel at a first temperature.

Aspect 3: The method of aspects 1 or 2, wherein the first isothermal temp is about 16 to about 48° C.

Aspect 4: The method of any one of aspects 1-3, wherein the first period of time is about 10 min to about 3 hours.

Aspect 5: The method of any one of aspects 1 to 4, further comprising combining the sample and the padlock probe in the reaction vessel and incubating the sample and the padlock probe at a second temperature for a second period of time before adding RNP, ligase, polymerase, and reporter DNA and incubating at the first temperature and first period of time in the reaction vessel.

Aspect 6: The method of aspect 5, wherein the second temperature is about 45 to about 99° C.

Aspect 7: The method of any of aspects 1-6, wherein the second period of time is about 1 to about 30 minutes.

Aspect 8: The method of any of aspects 1-7, wherein the polynucleotide sequence of the detection zone lacks a PAM sequence.

Aspect 9: The method of any one of aspects 1 to 8, wherein the cas12a enzyme is a Lba cas12a (cpf1).

Aspect 10: The method of any one of aspects 1 to 9, wherein the crRNA comprises a polynucleotide sequence complementary to a conserved sequence of Lba cas12a, a variable sequence of Lba cas12a, or both.

Aspect 11: The method of any one of aspects 1 to 10, wherein the ligase is a SplintR ligase or T4 ligase.

Aspect 12: The method of any one of aspects 1 to 11, wherein the polymerase is a Phi29 polymerase, Bst polymerase, or a Klenow Fragment.

Aspect 13: The method of any one of aspects 1 to 12, wherein the reporter DNA comprises: a polynucleotide, a detectable molecule, and a quencher, wherein the detectable molecule and quencher are linked to opposite ends of the polynucleotide and wherein the polynucleotide is configured to be cleaved by the Cas enzyme of the RNP upon activation.

Aspect 14: The method of any one of aspects 1 to 22, wherein the detectable molecule is one or more of FAM, FITC, Cy3, Cy5, HEX, TAMRA.

Aspect 15: The method of any one of aspects 1 to 14, wherein the reaction vessel further comprises one or more additional components selected from the group consisting of: deoxyribonucleoside triphosphates (dNTPs), bovine serum albumin (BSA), a reaction buffer, water, and combinations thereof.

Aspect 16: The method of any one of aspects 1 to 15, wherein the sample is from a subject having or suspected of having a pancreatic ductal adenocarcinoma (PDAC) and wherein the one or more microRNAs are one or more biomarkers of PDAC.

Aspect 17: The method of any of aspects 1-16, wherein the concentration of RNP is from about 0.1-10 nM.

Aspect 18: The method of any one of aspects 1 to 17, wherein the RNP is formed by incubating the cas12a enzyme and crRNA at a third temperature for a third period of time before placing in the reaction vessel.

Aspect 19: The method of aspect 18, wherein the third temperature is about 4 to about 48° C. and the third period of time is about 10 to about 180 minutes.

Aspect 20: A one-pot nucleic acid detection system for detecting a target polynucleotide in a sample, the system comprising: a set of isothermal detection components comprising: a Cas12a CRISPR-associated (Cas) enzyme; a ligase; a polymerase; and a reporter deoxyribonucleic acid (DNA) capable of producing a CRISPR-generated detectable signal or detectable molecule upon cleavage by the Cas enzyme.

Aspect 21: The system of aspect 20, further comprising a single reaction vessel configured to contain the elements of the system of aspect 20 in a single pot.

Aspect 22: The system of aspect 20 or 21, wherein the system further comprises one or more of: a heating element to maintain the reaction vessel at a temperature of about 60-90° C. and 25-40° C.; a portable, handheld computing device programmed to detect the detectable signal; and a support structure configured to hold one or more of the reaction vessel, the heating element, and the computing device in proximity to each other.

Aspect 23: The system of any one of aspects 20-22, further comprising a crRNA configured to form a ribonucleoprotein complex (RNP) with the Cas12a enzyme, wherein the crRNA is provided separately from the Cas12a enzyme or wherein the crRNA is pre-combined the Cas12a enzyme to provide a RNP in the system.

Aspect 24: The system of aspect 23, wherein the concentration of RNP in the system is from about 0.1-10 nM.

Aspect 25: The system of any one of aspects 23-24, further comprising a padlock probe comprising a ligation zone and a detection zone, the ligation zone comprising a polynucleotide sequence complementary to the target polynucleotide, and the detection zone comprising a sequence capable of hybridizing with the crRNA, the ligation zone further comprising a nick in the sequence of the ligation zone complementary to the miRNA target of interest, wherein the nick defines the 5′ and 3′ ends of the padlock probe, and wherein the 5′ end is phosphorylated.

Aspect 26: The system of any one of aspects 20 to 25, wherein the reporter DNA comprises a polynucleotide, a detectable molecule, and a quencher, wherein the detectable molecule and quencher are linked to opposite ends of the polynucleotide and wherein the polynucleotide is configured to be cleaved by the Cas enzyme of the RNP upon activation.

Aspect 27: The system of any one of aspects 20 to 26, wherein the detectable molecule is one or more of FAM, FITC, Cy3, Cy5, HEX, and TAMRA.

Aspect 28: The system of any one of aspects 20 to 28, further comprising one or more additional components selected from the group consisting of: deoxyribonucleoside triphosphates (dNTPs), bovine serum albumin (BSA), a reaction buffer, water, and combinations thereof.

Aspect 29: The system of any one of aspects 20 to 28, wherein the cas12a enzyme is a Lba Cas12a.

Aspect 30: The system of any one of aspects 20 to 29, wherein the crRNA comprises a polynucleotide complementary to a conserved sequence of Lba cas12a, a variable sequence of Lba cas12a, or both.

Aspect 31: The system of any one of aspects 20 to 30, wherein the ligase is a SplintR ligase or a T4 ligase.

Aspect 32: The system of any one of aspects 20 to 31, wherein the polymerase is a Phi29, Bst polymerase, or Klenow Fragment.

Aspect 33: The system of any one of aspects 20-32, further comprising a multi-well plate, wherein each well serves as a single reaction vessel configured to contain the elements of the system of aspect 20 in a single pot.

Aspect 34: A shelf-stable kit for detecting a target polynucleotide in a sample comprising the following components: a) a lyophilized Cas12a CRISPR-associated (Cas) enzyme and a lyophilized CRISPR-RNA (crRNA), wherein the crRNA is capable of hybridizing with the detection zone of the padlock probe, and wherein the crRNA and Cas enzyme are capable of forming a ribonucleoprotein (RNP) complex; b) a lyophilized ligase; c) a lyophilized polymerase; d) a lyophilized reporter; and instructions for combining lyophilized components a-d with a sample and a specific padlock probe, incubating the sample and the padlock probe at a first temperature for a first period of time, and detecting the detectable signal or molecule.

Aspect 35: The kit of aspect 34, wherein the kit further comprises a lyophilized isothermal amplification buffer.

Aspect 36: The kit of any one of aspects 34 or 35, further comprising a multi-well plate, wherein each well serves as a single reaction vessel configured to contain the lyophilized components of the kit in a single pot.

Aspect 37: The kit of aspect 36, wherein the lyophilized components are formed into a pellet and each well of the multi-well plate comprises a pellet comprising the lyophilized reaction components.

Aspect 38: The kit of any one of aspects 34 to 37, wherein the cas12a enzyme is a Lba cas12a.

Aspect 39: The kit of any one of aspects 34 to 38, wherein the ligase is a SplintR ligase or T4 DNA ligase.

Aspect 40: The kit of any one of aspects 34 to 39, wherein the polymerase is a Phi29, Bst polymerase, or Klenow Fragment.

Aspect 41: The kit of any one of aspects 34 to 40, further comprising a padlock probe, wherein the padlock probe comprises comprising a ligation zone and a detection zone, the ligation zone comprising a polynucleotide sequence complementary to the target polynucleotide, and the detection zone comprising a sequence capable of hybridizing with the crRNA, wherein the ligation zone of the padlock probe comprises a nick in the sequence of the ligation zone complementary to the miRNA target of interest, wherein the nick defines the 5′ and 3′ ends of the padlock probe, and wherein the 5′ end is phosphorylated.

Aspect 42: The kit of aspect 41, wherein the padlock probe is lyophilized.

Aspect 43: The kit of any one of aspects 34 to 42, wherein the detection zone comprises a polynucleotide that lacks a PAM sequence (TTTV, V=A, G, C).

Aspect 44: The kit of any one of aspects 34 to 43, wherein the crRNA comprises a polynucleotide complementary to a conserved sequence of cas12a, a variable sequence of cas12a, or both.

Aspect 45: The kit of any one of aspects 34 to 44, further comprising one or more additional components selected from the group consisting of: deoxyribonucleoside triphosphates (dNTPs), bovine serum albumin (BSA), a reaction buffer, water, and combinations thereof.

Aspect 46: The kit of aspect 45, wherein any one or more of the additional components are lyophilized.

Aspect 47: The kit of any one of aspects 34 to 46, wherein the reporter DNA comprises a polynucleotide, a detectable molecule, and a quencher, wherein the detectable molecule and quencher are linked to opposite ends of the polynucleotide and wherein the polynucleotide is configured to be cleaved by the Cas enzyme of the RNP upon activation.

Aspect 48: The kit of any one of aspects 34 to 47, wherein the detectable molecule is one or more of FAM, FITC, Cy3, Cy5, HEX, and TAMRA.

Aspect 49: The kit of any of the previous aspects wherein the concentration of RNP after combination of the kit with the sample and the padlock probe is from about 0.1-10 nM.

Aspect 50: A digital, one-pot nucleic acid detection system for detecting a target polynucleotide in a sample, comprising: the system of any of aspects 20-33 and/or a kit of any of aspects 34-49; a single reaction vessel configured to contain the components of the system in a single pot suitable for digital detection; and a heating element to maintain the reaction vessel at a temperature of about 60-90° C. and 25-40° C.

Aspect 51: A point-of-care system for visible detection of a target polynucleotide in a sample, the point-of-care system comprising:
the system of any of aspects 20-33 and/or a kit of any of aspects 34-49, wherein the reporter DNA comprises a polynucleotide, a first detectable molecule, and a binding moiety, wherein the detectable molecule and binding moiety are linked to opposite ends of the polynucleotide and wherein the polynucleotide is configured to be cleaved by the Cas enzyme of the RNP upon activation; and
a lateral flow visible detection kit compatible with the system, the lateral flow visible detection kit comprising a test strip comprising: a region comprising a reporter antibody specific for the first detectable molecule of the reporter DNA, the reporter antibody also comprising a second reporter molecule that produces a visible signal; a control line comprising a capture peptide capable of specifically binding the binding moiety of the reporter DNA; and a test line comprising a capture antibody specific for the first detectable molecule, wherein the capture antibody can only bind the first detectable molecule that has been cleaved from the first binding moiety.

Aspect 52: The point-of-care system of aspect 51, wherein the first detectable molecule is FAM and the reporter antibody is anti-FAM.

Aspect 53: The point-of-care system of aspect 51 or 52, wherein the second detectable molecule is Ab-coated gold nanoparticle.

Aspect 54: The point-of-care system of any one of aspects 51-53, wherein the binding moiety of the reporter DNA is biotin and the capture peptide in the control strip is streptavidin.

The following examples are put forth so as to provide those of ordinary skill in the art with a complete disclosure and description of how to perform the methods and use the compositions and compounds disclosed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

### One-Pot Endonucleolytically Exponentiated Rolling Circle Amplification by CRISPR-Cas12a Affords Sensitive, Expedited Isothermal Detection of MicroRNAs

### Overview

Existing CRISPR-based miRNA assays require multiple manual steps and lack the analytical performance of the gold standard, RT-qPCR. Radically deviating from the existing strategies, the present example describes an embodiment of a one-step, one-pot isothermal miRNA assay referred to here as “Endonucleolytically eXponenTiated Rolling circle Amplification with the dual-functional CRISPR-Cas12a” (EXTRA-CRISPR) to afford RT-PCR-like performance for miRNA detection. This system and method demonstrate high sensitivity and specificity of the EXTRA-CRISPR assay to detect miRNAs (miR-21, miR-196a, miR-451a, and miR-1246) in plasma extracellular vesicles, which allowed elucidation of an EV miRNA signature for detection of pancreatic cancer. The analytical and diagnostic performance of this one-pot assay were shown to be comparable with that of the commercial RT-qPCR assays, while greatly simplifying and expediting the analysis workflow. This technology can provide a tool to advance miRNA analysis and clinical marker development for liquid biopsy-based disease diagnosis and prognosis. Moreover, this simple and robust assay permitted direct coupling with a 3D printed smartphone-based portable device and the lateral flow assay for signal readout, demonstrating its potential adaptability to low-cost point-of-care diagnostics.

### Introduction

MicroRNAs (miRNAs) are endogenous and short non-coding single-stranded RNAs (18-23 nucleotides) that are involved in the post-transcriptional repression of messenger RNAs (mRNAs). Because they participate in various biological processes such as cell proliferation, differentiation, and cell death, dysregulated miRNAs are closely linked to the pathogenesis of diseases such as cancers.¹⁻⁴ miRNAs were originally studied in tissues, but they have also been discovered in the blood, urine, and other body fluids, either associated with ribonucleoprotein complexes or argonaute-2, or encapsulated in exosomes,⁵ making probing circulating miRNAs a promising strategy in liquid biopsy-based cancer diagnosis, prognosis, and monitoring.^{2, 5, 6} Despite the promise of miRNAs, applying their use to clinical practice remains a work in progress. One roadblock is the challenge in high-performance detection of miRNAs in biospecimens due to their short length, high sequence similarity within miRNA families, enormous concentration range in different cell types and biofluids, and complexity of associated origins and carriers.⁶⁻⁹ Therefore, there is a pressing need for ultrasensitive, specific, and robust bioassays and sensors to facilitate the development of clinically viable miRNA biomarkers of diseases.

Reverse transcription-quantitative polymerase chain reaction (RT-qPCR) has been the gold standard tool for miRNA detection. Distinct from long RNA species, such as mRNAs, short miRNAs necessitates a special RT process to incorporate extended sequences that facilitate PCR amplification and detection.^{10, 11} Stem-loop and polyadenylation (poly-A) RT assays are two commonly used approaches and broadly adapted in many commercial miRNA RT-qPCR kits.^{8, 10} Alternative to thermal cycling-based qPCR that requires sophisticated analytical procedures and instruments, many isothermal assays have been developed to advance miRNA detection,¹² including rolling circle amplification (RCA),¹³⁻¹⁵ exponential amplification reaction (EXPAR),^{16, 17} loop-mediated isothermal amplification (LAMP),¹⁸ hybridization chain reaction (HCR),^{19, 20} and catalytic hairpin assembly (CHA).^{21, 22} Despite their merits in simplicity and even instrument-free operation, these assays have drawbacks that limit widespread clinical application, such as non-specific amplification and high background of EXPAR and LAMP, and relatively slow kinetics and low sensitivity of HCR and CHA. Other standard technologies such as microarrays,²³ NanoString,²⁴ and sequencing²⁵ offer powerful tools for highly multiplexed miRNA profiling with high throughput and flexibility.^{26, 27} However, these methods demand sophisticated instruments, complex analytical workflow, high operational cost, and limited analytical performance, which hinders their widespread applications to clinical diagnostics, especially point-of-care testing.

Recently, CRISPR (clustered regularly interspaced short palindromic repeats) technologies have emerged as a versatile platform for developing the next-generation bioassays that combine the analytical performance of PCR and the ease of isothermal amplifications. The CRISPR-Cas12a/-Cas13a systems confer highly specific target recognition via binding with the Cas enzyme-crRNA complex and enzymatic signal amplification via collateral cleavage (trans-activity) of a fluorogenic probe by the Cas enzyme activated upon target binding.^{28, 29} A variety of CRISPR assays have been reported for DNA and viral RNA detection which normally require an additional PCR or isothermal pre-amplification step to achieve desirable detection sensitivity.³⁰⁻³³ Following the same strategy, sensitive CRISPR-based miRNA assays were also developed by incorporating pre-amplification of miRNA targets by various isothermal reactions, including RCA,³⁴⁻³⁶ LAMP,³⁷ and cascade amplification.³⁸ However, these methods involving two separate pre-amplification and CRISPR-mediated readout steps require multi-step manual operations, which not only leads to complicated assay workflow and extended turnaround time, but also increases the risk of analytical variations and false results due to human error, enzymatic degradation, and cross-contaminations. It was recently demonstrated that it is possible to combine isothermal amplification and CRISPR detection in a one-pot reaction via delicately engineering the primer designs and reaction conditions.^{32, 39-41} However, it remains unclear if such strategy can be adapted to develop one-step, one-pot CRISPR assays for miRNA sensing. Alternatively, CRISPR-mediated target recognition can be integrated with other signal transduction modalities, such as electrochemical,⁴² plasmonic,⁴³ and graphene field-effect transistor (gFET) sensors,⁴⁴ enabling amplification-free nucleic acid detection. However, these approaches are not truly comparable to RT-qPCR, due to either low sensitivity without pre-amplification⁴² or the limitations arising from highly specialized devices and instruments needed. Lastly, it is worth noting that most, if not all, of the existing CRISPR-based methods leverage on the trans-cleavage activity of Cas proteins, while the exploration of the specific cis-cleavage activity (the primary mechanism of CRISPR-Cas systems for gene editing) for biosensing remains largely untapped.

The present example reports a one-step, one-pot isothermal CRISPR-Cas12a assay termed “Endonucleolytically eXponenTiated Rolling circle Amplification with CRISPR-Cas12a” (EXTRA-CRISPR) for rapid, specific detection of miRNAs with RT-PCR-comparable sensitivity. FIGS. 1A-1B are schematic illustrations of the EXTRA-CRISPR system/assay. The EXTRA-CRISPR assay offers three major distinctions from the existing CRISPR-based biosensing methods. First, it presents the first strategy to simultaneously harness both cis-cleavage and trans-cleavage activities of the CRISPR-Cas12a system. Specifically, it exploits the specific cis-cleavage activity to transform conventional linear RCA to enable exponential amplification of target sequences, in addition to the trans-cleavage reaction for amplicon detection and signal amplification. Second, by engineering a modular padlock probe design and the reaction kinetics, multiple reactions can be incorporated for target-mediated ligation, RCA, Cas12a binding, and nucleolytic cleavage into one collaboratively coupled reaction network, creating a robust one-step, single-tube isothermal assay for miRNA analysis. Third, this one-pot isothermal miRNA assay affords comparable analytical performance with standard RT-qPCR, including high sensitivity with a single-digit fM detection limit, single-nucleotide specificity, and rapid and flexible turnaround (from 20 min to 3 h for the entire analysis depending on targets and samples). Lastly, the one-pot EXTRA-CRISPR technology vastly simplifies the assay workflow and negates the needs for specialized instruments, which provides an adaptable modality for point-of-care diagnostics.

As a proof-of-concept of potential applications, the EXTRA-CRISPR assay was adapted to quantifying miRNA biomarkers in extracellular vesicles (EVs) for liquid biopsy diagnosis of pancreatic ductal adenocarcinoma (PDAC). EVs, including exosomes of 50-150 nm in size, are emerging as a promising candidate for liquid biopsy because they selectively sort and transport cellular cargoes, such as proteins and nucleic acids, that mirror the physiological and pathological states of parental cells.⁴⁵⁻⁵⁰ EVs are considered as a major carrier of miRNAs in human biofluids and tumor-derived EVs offer a promising route to explore disease-specific miRNA signatures.⁴⁵⁻⁵² Using the EXTRA-CRISPR, highly sensitive and specific profiling of a panel of four miRNA markers (miR-21, miR-196a, miR-451a, and miR-1246) in EVs derived from cell lines and clinical plasma specimens was demonstrated. Based on the individual EV-miRNA tests, an EV signature (EV-Sig) was devised with a machine learning method to improve the diagnostic performance for pancreatic cancer. The analytical and diagnostic performance of the EXTRA-CRSPR tests were rigorously validated by parallel RT-qPCR analysis of the same clinical samples. These results demonstrate the present technology as a useful tool to advance miRNA detection and clinical development of miRNA biomarkers for liquid biopsy-based cancer diagnosis and prognosis.

### Results

### Mechanistic Studies of the EXTRA-CRISPR Assay

The present assay is designed to be a tri-enzymatic cascade that exploits the unique nucleolytic cleavage activities of the CRISPR-Cas12a system to create a new exponential isothermal amplification mechanism based on the robust linear RCA assay. The assay starts with the hybridization of a padlock probe with the miRNA target in the one-pot reaction, which can be further enhanced by adding a rapid denaturing and annealing step in the sample preparation process prior to analysis, as illustrated in FIG. 1A. The padlock probe is a 5′-phosphorylated single-strand DNA engineered to encompass two modular sequences: a ligation zone bridging the 5′- and 3′-termini with a complementary sequence to target miRNA, and a detection zone whose complementary sequence can be recognized by Cas12a-crRNA ribonucleoprotein complexes (RNP). Upon mixing with all other assay reagents in a tube, the target-splinted padlock probe will be ligated with the SplintR ligase to form a circular template for isothermal RCA reaction. Driven by phi29 DNA polymerase, RCA continuously extends target miRNA to a long linear concatemer with repeatedly complementary copies of the padlock. The Cas12a RNP pre-formed in the solution will bind to the detection zones on the long concatemer, which activates the trans-cleavage activity of Cas12a enzyme to non-specifically cut the fluorophore quencher (FQ)-labeled single-stranded DNA (ssDNA) reporters to produce fluorescence signal (FIG. 1B). In the meantime, the activated RNP can also cut the linear RCA product into short fragments via its cis-cleavage function. These fragments contain single or multiple complementary copies of the padlock and thus can serve as new primers to trigger many secondary RCA reactions. Such collaboratively coupled linear DNA polymerization and Cas12a cis-cleavage can be repeated continuously to generate the chain reactions converting conventional linear RCA to an exponential amplification assay (FIG. 1B). The post-cleavage RNP complexes may also cause collateral cut of the ssDNA reporters and thus further promote CRISPR signal amplification to enhance the detection sensitivity.

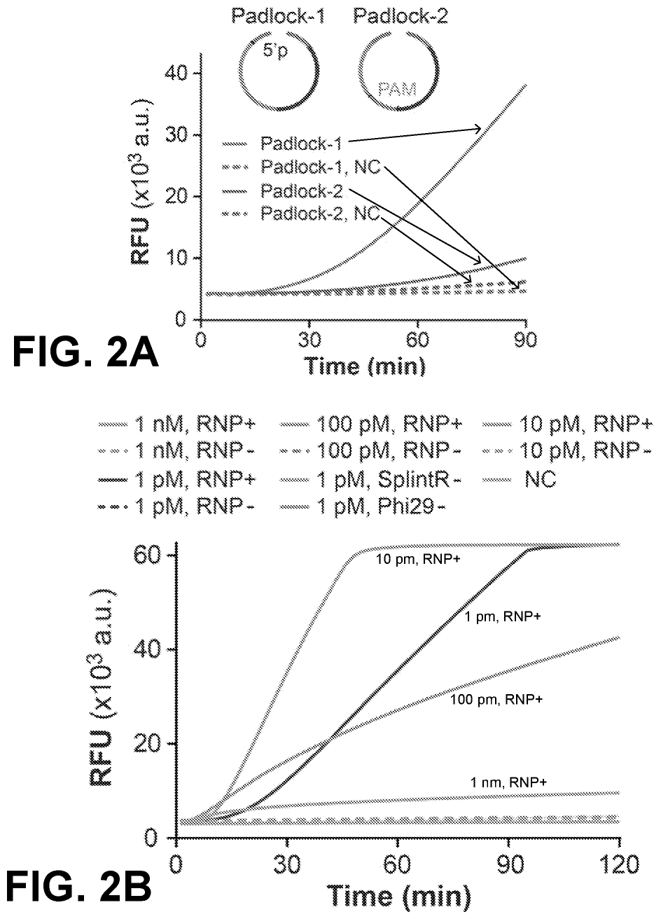

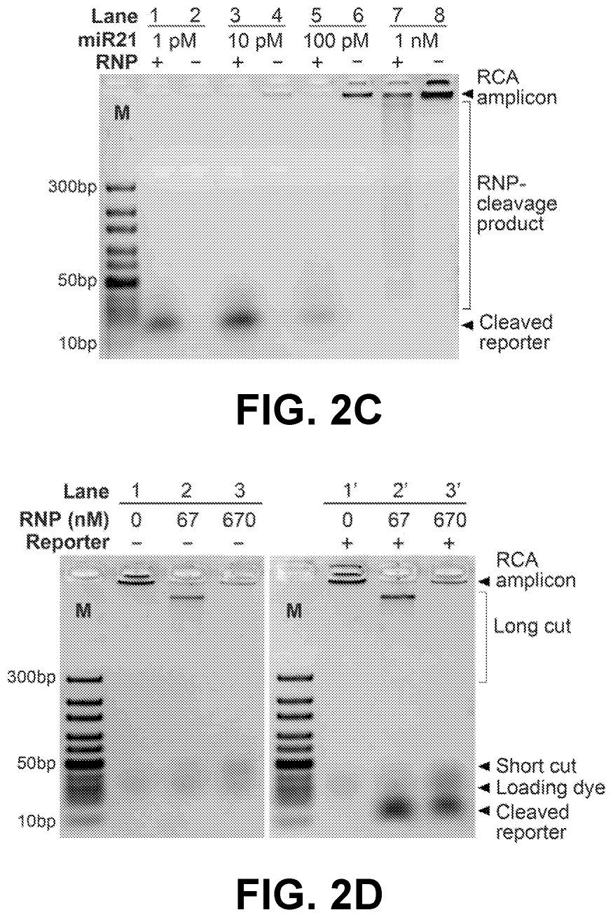

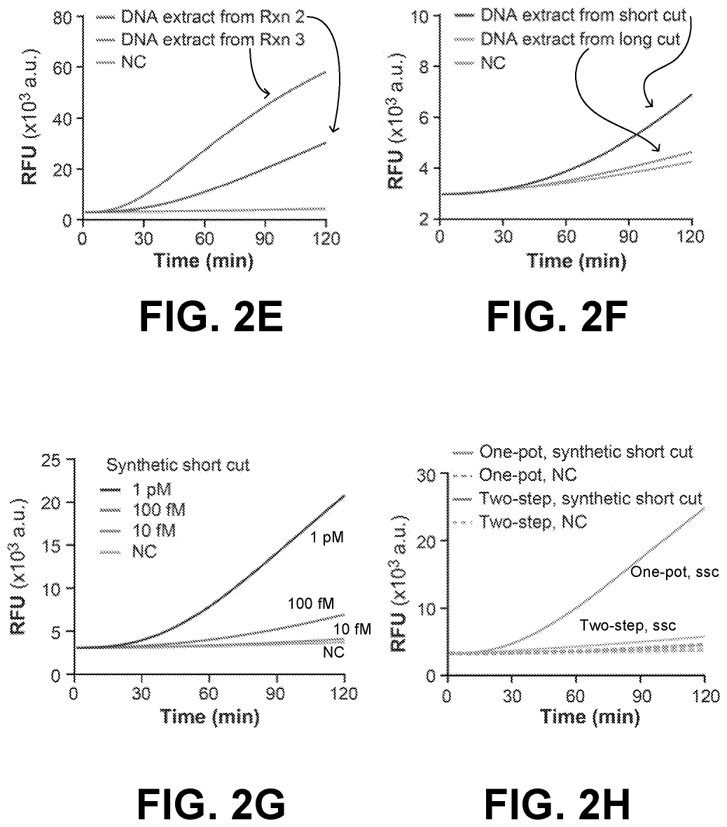

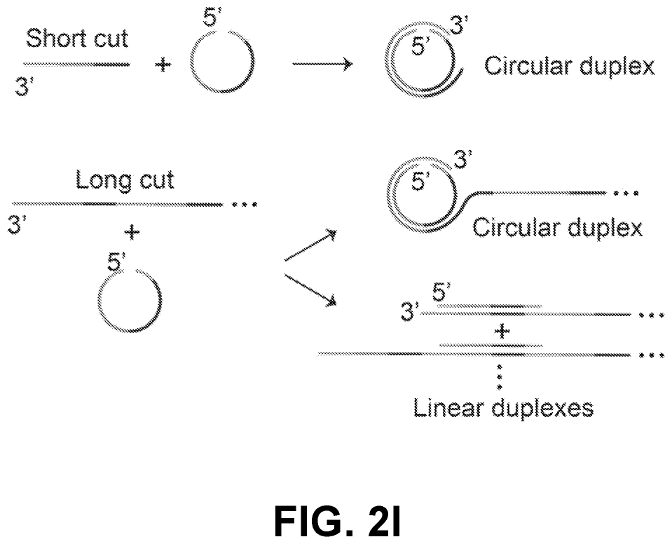

The development and mechanistic studies of EXTRA-CRISPR were conducted using miR-21 as the model target, which has been found overexpressed in various human tumors.⁵³ A key module in the padlock probe design, the CRISPR detection zone, was verified by detecting its complementary strand with a CRISPR-Cas12a only assay. A limit of detection (LOD) at the one picomolar (pM) level was obtained (data not shown, but available in supplementary information for Yan, et al., One-Pot Endonucleolytically Exponentiated Rolling Circle Amplification by CRISPR-Cas12a Affords Sensitive, Expedited Isothermal Detection of MicroRNA; bioRxiv; doi; https://doi.org/10.1101/2022.05.01.490215, which is hereby incorporated herein by reference, in its entirety, as well as for other results not shown here), in line with the reported performance for preamplification-free CRISPR-Cas detection.^{31, 42} The present EXTRA-CRISPR assay is expected to produce both single-stranded RCA amplicon and the amplicon-padlock duplex both containing the crRNA-complementary sequences. For Cas12a enzymes, RNA-guided binding with a dsDNA activator requires a protospacer-adjacent motif (PAM) to activate Cas12a for both specific cis-cleavage and effective non-specific ssDNA trans-cleavage, whereas a ssDNA does not need the PAM but yields less catalytic activity for trans-ssDNA cutting.³¹ Hence, the effects of CRISPR-Cas12a in the present one-pot assay was first examined by tuning its activities with the PAM sequence in the padlock. As shown in FIG. 2A, the PAM-free Padlock-1 unexpectedly resulted in a significantly higher reaction rate and signal level as compared to the Padlock-2 with a PAM sequence, indicating an inhibitory effect of the PAM in the padlock for the one-pot assay. This result implies that establishing optimized equilibrium among the RCA and CRISPR-Cas12a cleavage reactions to catalyze efficient target amplification for which a PAM-free padlock is preferred, as further examined below.

To facilitate the mechanistic studies, the EXTRA-CRISPR reactions were first conducted with Padlock-1 at high miR-21 concentrations (1 pM to 1 nM) to permit both real-time fluorescence detection and gel electrophoresis analysis of the reaction products. FIG. 2B shows that the present tri-enzyme assay enormously increases the detection signal with 1 pM miR-21 compared to the Cas12a only detection (data not shown). In the control reactions with one of three enzymes left out each time, no fluorescence signal was detected, verifying the role of each enzyme in the one-pot EXTRA-CRISPR system. The signal intensity was observed to rise with the miR-21 concentration increased to 10 pM, but then to decrease at 100 pM and 1 nM (FIG. 2B), indicating the dynamic coupling of competing reactions associated with Cas12a in this tri-enzyme assay. The products of these reactions were analyzed with agarose gel electrophoresis. In the absence of Cas12a RNP, a band of high molecular weight DNA was detected at the edge of the sample wells, and the DNA amount increased with the miR-21 input (FIG. 2C), which confirms successful miR-21 amplification by ligation-assisted RCA. No cleaved reporter was detected on the gel, consistent with the negative fluorescence detection seen in FIG. 2B. With RNP added, the one-pot reaction at a relatively low miR-21 concentration (1-100 pM) resulted in a barely detectable band of long RCA product and a weak band of small molecular weight DNA (FIG. 2C, lanes 1, 3 and 5), which can be presumably attributed to relatively complete cleavage of long DNA product by the activated RNP. In addition, a clear fluorescent gel band of collaterally cleaved reporter by Cas12a was detected with the intensity being enhanced at the target concentration of 10 pM and then reduced at 100 pM (FIG. 2C, lanes 1, 3 and 5), which agrees with the real-time detection results (FIG. 2B).

When the miR-21 concentration was further increased to 1 nM (FIG. 2C, lane 7), the long RCA amplicon was clearly detected, excluding the inhibition of RCA reaction as the main factor for signal suppression observed at high target concentrations. The band of long RCA amplicon was much weaker than that for the RNP-free reaction (FIG. 2C, lane 8) and largely smeared, which verifies Cas12a cleavage of the RCA product. Compared to the cleaved DNA bands observed for the lower target concentrations, the broad smearing indicates much less effective cleavage by the excessive DNA amplicon produced with 1 nM miR-21. While the activated Cas12a can cause both cis- and trans-cleavage of the RCA amplicon, the observed smearing bands should be mainly from the trans-cleavage product, because our standard gel electrophoresis assay was not sensitive enough to detect the low-level cis-cleaved DNA produced with 1 nM of RNP. Moreover, the cleaved-reporter band became indiscernible when the target concentration increased to 1 nM (FIG. 2C). Such competitive cutting of reporter versus RCA amplicon can be attributed to the non-specific ssDNase activity of Cas12a that leads to preferential cut of the RCA amplicon when a high target input initiates extremely fast RCA reaction to produce significantly more ssDNA products than the reporter. The competing effect of Cas12a trans-activity was verified by conducting a simple ssDNA cleavage reaction in which a transition from the reporter-dominant to padlock-dominant cleavage was observed upon the descending reporter-to-padlock ratio (data not shown). In the assay, the padlock concentration was only 1/10 of the reporter and thus the padlock degradation by Cas12a trans-cutting should have minuscule effect on the EXTRA-CRISPR reaction. Together, these results suggest that the assay of the present disclosure can produce a favorable RCA amplicon-to-reporter ratio over a broad range of target input (up to ˜100 pM, equivalent to 109 copies per 20-μL reaction), enabling quantitative trans-cleavage of the fluorogenic reporter for accurate miRNA detection.

For comparison, the experiments for 1 pM to 1 nM miR-21 discussed above were repeated with Padlock-2. Data is not shown, but the observed reaction kinetics appeared to be much slower than that with Padlock-1 (FIG. 2B), and significant amplification suppression occurred at a higher miR-21 concentration (1 nM vs. 100 pM). Gel analysis of these reactions (data not shown) detected a pattern of RNP-cleavage products and cleaved reporter similar to that obtained with Padlock-1 (FIG. 2C). Consistent with the real-time fluorescence detection, the gel bands of cleaved reporter were much weaker than that for Padlock-1, verifying the inhibitory effect of the PAM on the amplification efficiency of our one-pot assay. This effect can be presumably attributed to the PAM-mediated RNP binding with the amplicon-padlock duplexes that activates both cis-cutting of the circular templates for RCA and rapid non-specific degradation of all ssDNA species including the cleaved amplicons for the secondary RCA.s^{31, 39, 54} Indeed, compared to the Padlock-1 reaction (FIG. 2C), the Padlock-2 reaction was seen to produce relatively strong gel bands of low-molecular-weight RNP-cleavage products with respect to the reduced bands of cleaved reporter. Overall, this comparative study should support the role of the engineered PAM-free Padlock-1 in tuning the dual reactivities of CRISPR-Cas12a to drive exponential RCA of miRNA.

To further assess the Cas12a reactivities in the EXTRA-CRISPR reaction, a two-step assay was conducted in which the ligation-assisted RCA was first performed, followed by treating the amplicon with Cas12a RNP of variable concentrations. The reactions were run with the high concentrations of miR-21 (1 nM) and RNP (up to 670 nM) to facilitate the detection of both cis- and trans-cleaved DNA products. FIG. 2D shows that when 67 nM RNP was used, the long DNA amplicon produced by RCA (lanes 1 and 1′) were partially cleaved to yield a smeared band of long fragments and a band of short fragments (lanes 2 and 2′) which are thereafter referred to as the long and short cuts, respectively. This observation indicates enhanced cleavage of the RCA amplicon by more RNP in comparison to the assay shown in FIG. 2C (lane 7). Increasing the RNP concentration to 670 nM led to apparently complete digestion of the long cut into the short cut (lanes 3 and 3′). At these high levels of RNP over ssDNA substrate, the bands of trans-cleaved reporter were also detected (FIG. 2D, lanes 2′ and 3′), which is consistent with the observations for the one-pot reactions in which low levels of RCA amplicon were produced (FIG. 2C, lanes 1-4).

A distinct observation in the two-step reactions with high-concentration RNP was an intense band of long cut (FIG. 2D, lanes 2 and 2′) that was barely detectable with the low level of RNP (FIG. 2C, lane 7). It was hypothesized that this intense long cut band is mainly produced by the cis-activity of RNP and thus the short cut band should also contain a considerable amount of the small fragments of cis-cleaved RCA amplicon. These cis-cleavage products contain complementary copies of the padlock and may serve as new primers for secondary RCA reactions to initiate exponential amplification of target miRNA. To test this, the ability of CRISPR-cleaved RCA products to exponentiate the linear RCA reaction was investigated. Since the ssDNA amplicon of RCA is chemically different from miRNA, it was first verified that the DNA version of miR-21 yields comparable amplification efficiency with miR-21 for the EXTRA-CRISPR reaction (data not shown). The EXTRA-CRISPR assays were performed using the DNA extracted from the two-step reactions tested in FIG. 2D as the target. As seen in FIG. 2E, significantly higher amplification was obtained with the DNA extract that contains mostly the short cut (FIG. 2D, lane 3) than that containing both short and long cuts (FIG. 2D, lane 2).

To further examine such differential reactivity of cis-cleaved RCA products, DNA was extracted from the separated gel bands of the short and long cuts in the lane 2 of FIG. 2D were input as the targets for the EXTRA-CRISPR assays. Despite its larger quantity as detected on gel, the long-cut extract appeared to weakly trigger the tri-enzymatic reaction, while the short cut yielded much faster reaction kinetics and higher amplification signal (FIG. 2F). The low molecular weight of the short cut band observed on gel suggests that the cis-cleaved fragments in the band roughly correspond to one monomeric unit of the RCA concatemer with a length of 61 nucleotides (data not shown). Thus, a synthetic ssDNA of the unit sequence was assessed as the input for the EXTRA-CRISPR reaction. FIG. 2G demonstrates the quantitative titration of this synthetic short cut mimic down to a concentration of 10 fM, >100-fold lower than the LOD of the preamplification-free Cas12a assay. The one-pot assay with the synthetic short cut was seen to yield notably higher signal intensity than the two-step assay involving linear amplification of the synthetic short cut at the same concentration (1 pM, FIG. 2G), indicating the high efficiency of the short cut to trigger exponential RCA. Collectively, these results should verify the major contribution of the short cis-cleaved amplicon to exponentiating linear RCA over the long cis-cleaved ones, which may be explained by their length-dependent binding with the padlock probe as depicted in FIG. 2I. The unit sequence only binds with the termini of a padlock to form a circular duplex that initiates the exponential amplification. In contrast, the cis-cleaved long fragments may cause two competing effects via: 1) circularizing the padlock with the 3′-end complementary site to initiate RCA, and 2) hybridizing the padlock probes with other binding sites to form linear duplexes, which leads to the termination of exponential reaction. A long fragment has more padlock binding sites along the strand than at the terminal and thus the probability to form linear duplexes is higher than that for forming circular probes. Therefore, the ability of long cut fragments to initiate exponential RCA can be largely suppressed (FIG. 2F). Overall, the findings above demonstrate dynamic coupling of the trans- and cis-cleavage activities of Cas12a in the EXTRA-CRISPR assay, which enables exponential amplification of the target.

### One-Pot Chemistry Amplifies the Performance of Stepwise Combined Reactions

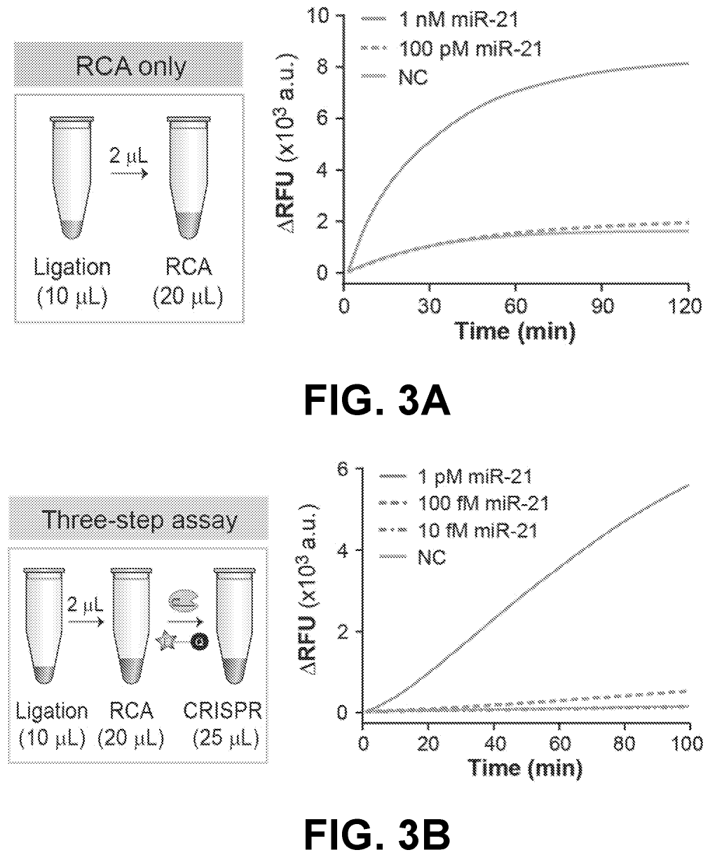

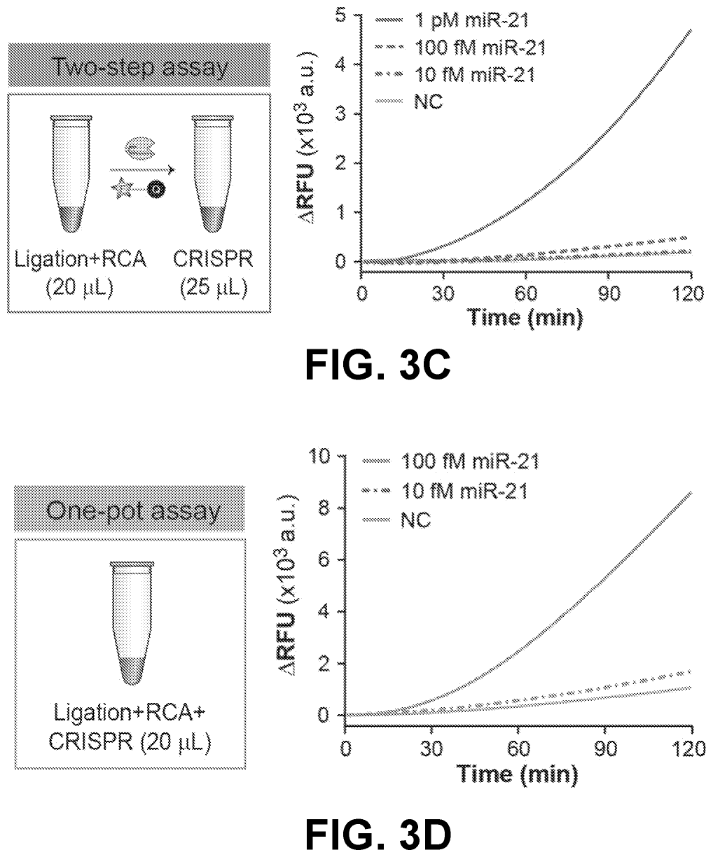

To directly assess the impact of dual-activity CRISPR on the tri-enzyme reaction network, miR-21 detection using the ligation-assisted RCA, tandem RCA and CRISPR, and one-pot EXTRA-CRISPR assays were compared under otherwise the same reaction conditions (see methods). The conventional assay included two sequential reactions of ligation and RCA affords a high LOD at the ˜100 pM level for miR-21 (FIG. 3A). Tandem combination of RCA amplification with the specific and powerful Cas12a-based signal amplification vastly improved the sensing sensitivity to detect miR-21 below 100 fM (FIG. 3B), which is in line with the performance reported with a similar three-step RCA-Cas12a assay.³⁶ Based on this three-step tandem assay, a simplified version was tested that combines ligation and RCA in one pre-amplification reaction followed by the Cas12a-powered fluorogenic readout. While simplifying the workflow, this two-step RCA-CRISPR-Cas12 approach appeared to slightly compromise the detection sensitivity and speed as indicated by the lower signal detected over a longer reaction time (FIG. 3C). This can be due to that the reaction conditions optimized for one enzyme may be suboptimal for others and thus the overall system. In contrast, the one-pot assay was able to overcome this challenge and confer sensitive detection of as low as 10 fM miR-21 using a protocol which notably outperforms the three-step assay based on linear RCA (FIG. 3D).

Given that the isothermal assays investigated here combines continuous RNA replication and fluorogenic signal amplification, their overall amplification efficiency was evaluated by quantifying the number of fluorescent probes produced per input miRNA template (equivalent to amplification fold, data not shown). The two-step RCA-CRISPR assay was estimated to yield 5.8×10⁴- and 5.9×10⁴-fold amplification at the 10 and 100 fM miR-21 input in 2 hours, respectively. These values are higher than the amplification efficiency of linear RCA by phi29 polymerase which was previously reported to be ˜1,000-2,000 replicates of one circular template per hour, owing to the Cas12a-based signal amplification.⁵⁵⁻⁵⁷ The overall amplification efficiency for the EXTRA-CRISPR assays with 10 and 100 fM miR-21 was estimated to be 1.6×10⁶ and 1.7×10⁶ FAM probes per template, respectively, which is ˜30-fold higher than that of the two-step assay. It is worth noting that such comparison of the overall amplification efficiency may largely underestimate the miRNA replication efficiency of the one-pot assay over the two-step assay, because the signal generation reaction in the one-pot assay occurs with the Cas12a target that increases from a very low level over the 2-hour reaction, while in the two-step assay the second reaction for signal generation starts with a vast quantity of the Cas12a target produced by the first RCA step. Overall, these comparative studies of different assay modes support the collaborative coupling of trans- and cis-activities of CRISPR-Cas12a in our assay to drive exponential RCA and fluorogenic signal amplification simultaneously. The dynamics of the coupled tri-enzyme reaction can be affected by several major factors, which were systematically optimized as detailed below.

### Optimization and Characterization of the EXTRA-CRISPR Assay

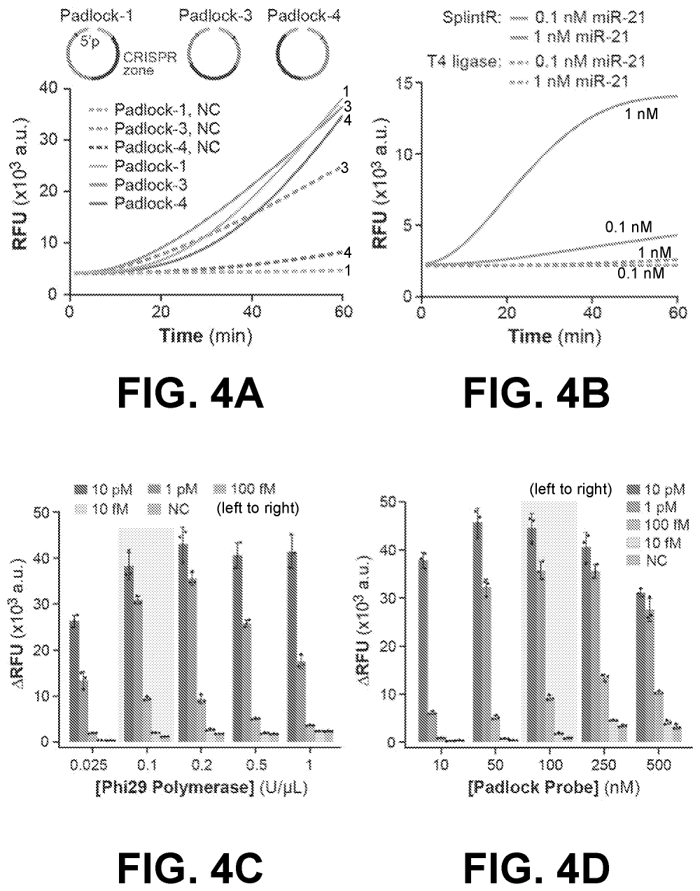

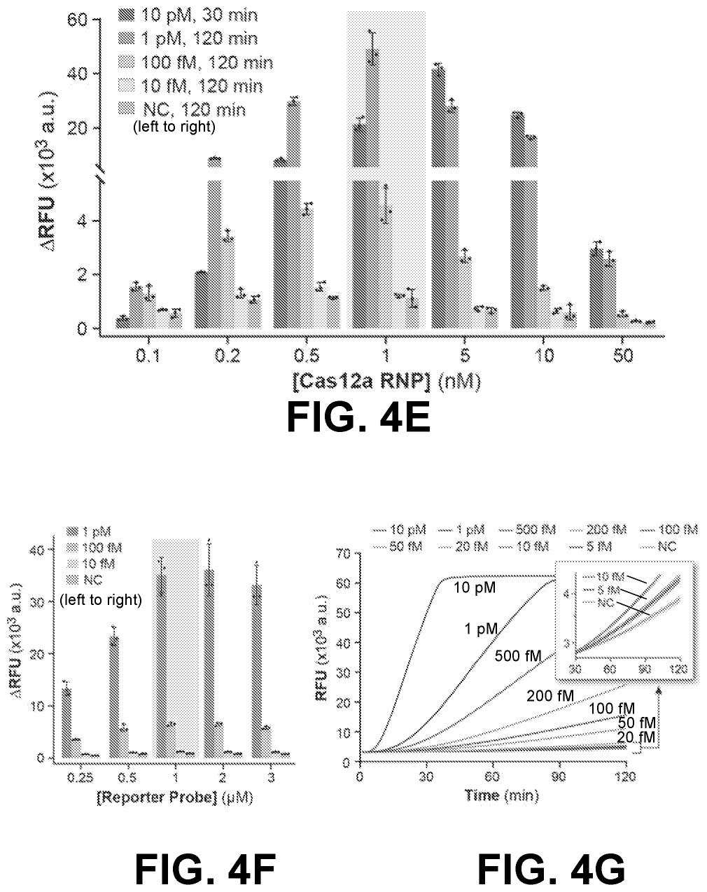

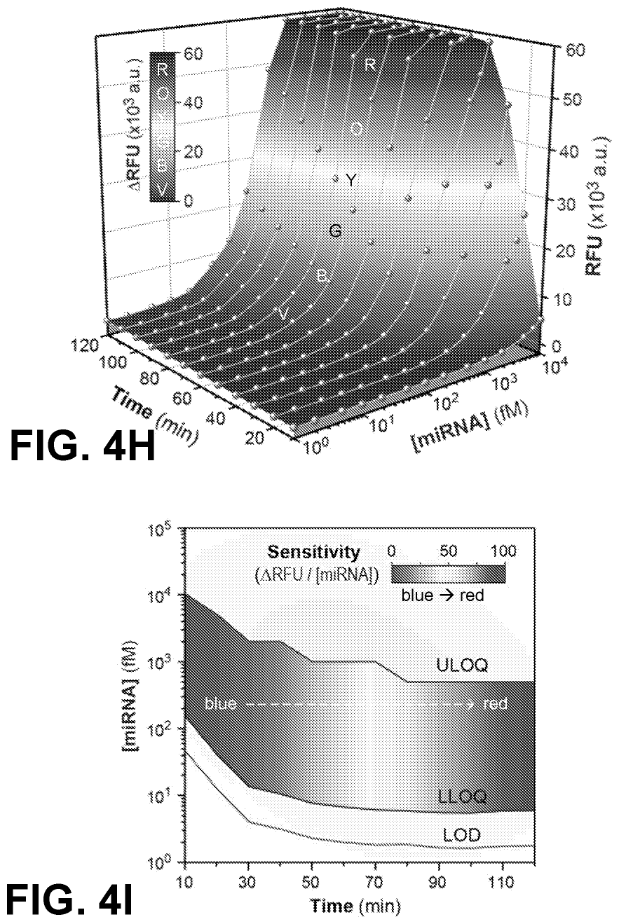

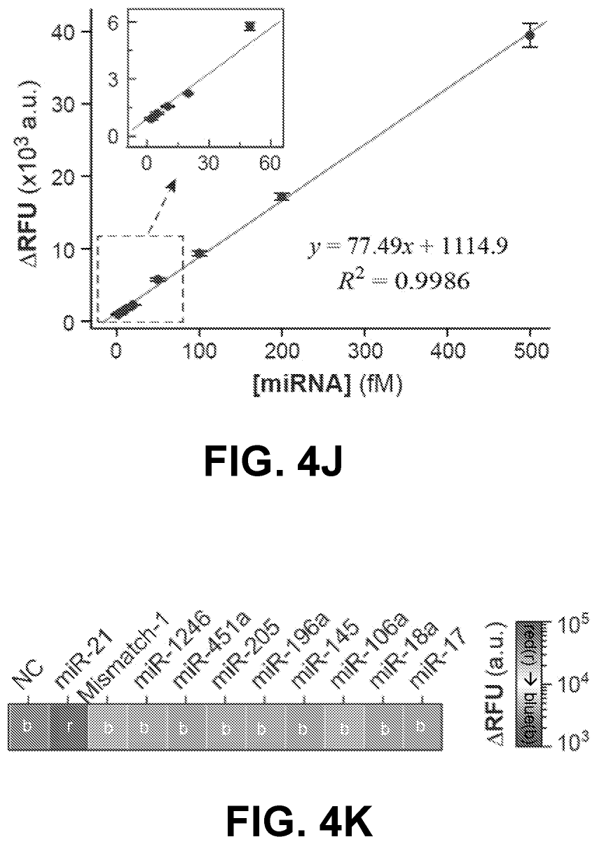

As discussed above, the padlock probe of the present disclosure is engineered with a CRISPR detection module which guides RNP cis-cleavage of RCA amplicon to generate new primers for secondary RCA. Moreover, insertion of this critical module affects the overall sequence of padlock probe that is an important factor governing the efficiency of ligation and RCA reactions.⁵⁸⁻⁶⁰ To assess these effects and optimize the padlock probe design, three padlock probes were constructed with the CRISPR detection zone located in the right (Padlock-1), middle (Padlock-3), and left (Padlock-4) of the sequence. As displayed in FIG. 4A, Padlock-1 constantly generated the highest detection signal and the lowest background level over an hour of reaction, indicating its advantage to enact efficient and specific EXTRA-CRISPR reactions. It is speculated that such effect may arise from the undesired secondary structures of the padlock probes that reduce the hybridization affinity and specificity for ligation and RCA, because oligonucleotide analysis showed that Padlock-3 and Padlock-4 can form more stable hairpins. To further test these effects, a set of variants of Padlcock-1 were designed by modifying the sequence outside the ligation and detection modules to intentionally induce more complex and stable hairpin structures and self-dimers (data not shown). Compared to the original Padlock-1 design, Padlock-1-1 with 3 modified nucleotides resulted in a largely reduced amplification signal and higher non-specific background. With more nucleotides altered to form increasingly stable hairpin and self-dimer structures, these variants led to deteriorating signal-to-background ratio, complete inhibition of specific amplification (Padlock-1-3), and greater non-specific background (Padlock-1-4). These results confirm the impact of the secondary structures of padlock probe on the one-pot reaction. There are other possible effects associated with the location of the detection zone, such as the steric hindrance that can affect the binding of three enzymes onto the circular template/padlock complexes.

Ligation of the padlock annealed to a miRNA splint is the initiating enzymatic reaction in the EXTRA-CRISPR cascade. Compared to DNA-DNA helices, RNA-splinted hybrid helices are known to be much less efficient substrates for DNA ligases, including T4 DNA ligase that is widely used in RCA assays.⁵⁹ To obtain an efficient ligation reaction, T4 DNA ligase was compared to the PBCV-1 DNA ligase, also commercially branded as SplintR ligase, which was reported to provide a much higher affinity (˜300-fold K_{M}) and turnover rate (20-fold k_{cat}) for RNA-splinted DNA substrates.⁵⁹ T4 DNA ligase appeared to be ineffective to trigger the one-pot assay, whereas SplintR ligase dramatically expedited the reaction kinetics and enhanced the signal intensity (FIG. 4B), indicating the importance of ligation to the overall reaction kinetics and efficiency. In addition, SplintR ligase confers good stability in the assay performance over a 20-fold change of enzyme quantity (2.5 to 50 units per reaction, data not shown). To further optimize the ligation reaction, the enhancement of miRNA/padlock probe hybridization was investigated by a quick denaturing and annealing step (FIG. 1A) which is a commonly practiced sample treatment method to promote nucleic acid hybridization in bioassays. This additional pre-treatment of RNA samples by 5 min denaturing and ˜1 min annealing improves the amplification efficiency while reducing the non-specifical background, compared to the original assay protocol. The denaturing step was found to remain effective when shortened to 10 seconds. Nevertheless, the final workflow implements a 6-min pre-treatment step to ensure the consistent assay performance without notably extending the total turnaround time.

Next, optimization of the major RCA-related components for the EXTRA-CRISPR assay was approached. It was found that Cas12a RNP can work effectively in the SplintR buffer with bovine serum albumin (BSA) (data not shown) and the SplintR buffer greatly outperforms the phi29 buffer. BSA was found to be an additive that can effectively augment the amplification efficiency in a proper concentration range (about 0.1-0.3 mg/mL). Two additional additives previously used to promote SplintR ligation, Mn²⁺ ion⁵⁹ and single-stranded DNA binding protein (SSB),⁶¹ were tested as well. Mn²⁺ ion appeared to dramatically increase non-specific background (data not shown) while adding SSB tends to suppress the EXTRA-CRISPR reaction, hence Mn²⁺ ion and SSB are not used in the following optimizations. The phi29 polymerase concentration was optimized which is a key factor to achieving the balanced RCA and CRISPR-Cas12a cleavage reactions to catalyze exponential amplification, as reasoned before. Similar to the effect of miRNA input (FIG. 2C), increasing phi29 polymerase quantity will promote RCA reaction to generate higher detection signal; but excessive RCA amplicon can suppress trans-ssDNA cutting of reporter by RNP, resulting in reducing signal intensity. The presence of an optimal polymerase concentration was experimentally observed, which displayed a shift to the higher level for lower miRNA input (FIG. 4C). Similar peaking behavior was observed for the padlock probe as well because higher padlock concentration can enhance the efficiency for target binding and subsequent RCA (FIG. 4D). Given the low abundance of miRNAs in many biological samples, including EVs, the concentrations of 0.1 U/μL and 100 nM for phi29 polymerase and the padlock, respectively, were selected which yielded the highest signal against the background for the range of 10 fM to 1 pM miR-21.

Optimization of the CRISPR components in the one-pot reaction started with assessing Cas12a RNP at a typical concentration of 50 nM used in the standard cas12a assays.³¹ However, this established assay condition led to poor signals for our assay (FIG. 4E), because excessive RNP can cause a termination effect, i.e., cis-cleavage of RCA amplicon-padlock duplexes and non-specific trans-cutting of the padlock, reducing the efficiency of both target amplification and fluorogenic CRISPR readout (FIG. 2). Thus, a wide range of Cas12a RNP concentrations from 0.1 to 50 nM were screened to find a condition that maximizes the desired catalytic effect against the adverse termination effect of CRISPR-Cas12a in the one-pot exponential amplification reaction. The assay signal displayed a peaking trend as a function of the RNP concentration depending on the target input and the optimal range was narrowed down to 0.5 to 1 nM for the targeted miRNA concentration range of 10 fM to 1 pM (FIG. 4E). This shows that not only can indiscriminate ssDNase activity of Cas12a cut the reporter but also degrade the padlock and linear RCA amplicon to inhibit the exponential amplification. Thus, increasing the reporter concentration would kinetically improve fluorogenic signal amplification and thermodynamically enhance the amplification efficiency by mitigating the inhibitory ssDNA cutting, while excessive reporter may raise the background signal. Indeed, the one-pot assay was seen to yield an ascending signal when the reporter concentration was increased from 25 nM to 1 μM (FIG. 4F). This effect was most dramatic at the high miRNA input (1 pM), which is consistent with the observed competitive Cas12a trans-cutting of ssDNAs (FIG. 2C). With other optimized variables, a combination of 1 nM RNP and 1 μM reporter were selected to maximize the signal to noise ratio of our one-pot assay. Collectively, these optimization studies revealed the unique dynamics of our assay which corroborates synergistic coupling of the enzymatic reactions via harnessing the dual activities of CRISPR-Cas12a.

Lastly, the analytical performance of the EXTRA-CRISPR assay was systematically calibrated with serial dilutions of synthetic miR-21 using the optimized protocol. FIG. 4G displays the typical curves for real-time detection over a reaction time of 2 h. The titration curves of signal versus concentration were also plotted at various time points, which show a strong dependence of the assay performance on the reaction time (FIG. 4H). To quantitatively evaluate the impact of reaction time on the assay performance, the analytical figures of merit were computed including LOD, sensitivity (the slope of linear calibration curve), and linear dynamic range defined by the lower limit of quantification (LLOQ) and upper limit of quantification (ULOQ), which are graphically presented in FIG. 4I. With 20-min assay time, our method conferred a LOD of 12.3 fM which vastly outperforms the three-step assay³⁶ reporting a LOD of 34.7 fM with a total of 4.5 h for both RCA and Cas12a reactions (see Table 1 below). Extending the reaction time improves the LOD and shifts the linear dynamic range down until reaching a minimal LOD of 1.64 fM with a linear range from 5.47 fM to 500 fM (R²=0.9986, FIG. 4J) at 100 min. Moreover, the calibration sensitivity of the assay can also be improved over time to better discriminate the small variations in target concentration.

TABLE 1
Comparison of EXTRA-CRISPR with other exemplary miRNA detection methods

| | Pre- | | One-pot | # major | Assay | | | Cost |
|---|---|---|---|---|---|---|---|---|
| Method | amplification | Cas function | reaction | components | time | | LOD | per test |
| Cas12a-SCR² | RCA to generate | Fluorogenic readout | No, | 4 enzymes, | 6 | h | miR-21: 47 fM | $1.9 |
| | pre-crRNA | by trans-cleavage | 3 steps | 6 probes | | | | |
| Cas12a-TCA³ | HRCA followed by | Fluorogenic readout | No, | 4 enzymes, | 7 | h | miR-21: 1 fM | $2.7 |
| | transcription to | by trans-cleavage | 3 steps | 7 probes | | | | |
| | generate pre-crRNA | | | | | | | |
| Cas12a-mediated | Ligation-triggered | Fluorogenic readout | No, | 3 enzymes, | 3.5 | h | let-7a: 21.9 fM | $3.5 |
| cascade | transcriptional | by trans-cleavage | 3 steps | 8 probes | | | | |
| amplification⁴ | amplification and | | | | | | | |
| | DNAzyme cleavage | | | | | | | |
| | to generate crRNAs | | | | | | | |
| Cas12a-enhanced | Ligation and linear | Fluorogenic readout | No, | 3 enzymes, | 4.5 | h | miR-21: 34.7 fM | $3.7 |
| RCA⁵ | RCA of target | by trans-cleavage | 3 steps | 3 probes | | | | |
| RCA-assisted | Ligation and linear | Fluorogenic readout | No, | 3 enzymes, | 3.5 | h | miR-21: 90 fM | N/A |
| CRISPR/ | RCA of target | by Cas9 cleavage | 4 steps | 3 probes | | | | |
| Cas9 cleavage | | | | per target | | | | |
| (RACE)⁶ | | | | | | | | |
| Cas13a-powered | Amplification- | trans-cleavage of | No, | 2 enzymes, | 4 | h | miR-19b, | N/A |
| electrochemical | free | affinity probe for | Multiple | 2 probes, | | | miR-20a: 10 pM | |
| microfluidic | | indirect | | 1 antibody, | | | | |
| sensor⁷ | | electrochemical | | 1 sensor chip | | | | |
| | | detection | | | | | | |
| CRISPR/Cas12a- | Ligation and LAMP | Fluorogenic readout | No, | 3 enzymes, | 1 | h | let-7a: 0.1 fM | $1.0 |
| Assisted Ligation- | for exponential | by trans-cleavage | 3 steps | 6 probes | | | | |
| Initiated LAMP⁸ | target | | | | | | | |
| | amplification | | | | | | | |
| EXTRA-CRISPR | No | Cis-cleavage of RCA | Yes | 3 enzymes, | 1.5 | h | miR-21: 1.64 fM; | $0.6 |
| | | amplicon to create | | 3 probes | | | miR-196a: 1.35 fM; | |
| | | secondary templates; | | | | | miR-451a: 4.14 fM; | |
| | | fluorogenic readout | | | | | miR-1246: 7.96 fM | |
| | | by trans-cleavage | | | | | | |
| Ligation-based | Pre-ligation | N/A | No | 2 enzymes, | | | miR-21: 20 aM | N/A |
| ddPCR⁹ | is needed | | | 2 probes, | | | | |
| | | | | 2 primers | | | | |
| Micro- | No | N/A | No | 2 probes | | | miR-208: 10 fM | N/A |
| array¹⁰ | | | | | | | | |
| Nano- | No | N/A | Yes | 1 probe | | | miR-375: 0.13 pM; | N/A |
| pore sensing¹¹ | | | | | | | miR-141: 0.1 pM | |

It was seen that the signals obtained with high miR-21 concentrations (1-10 pM) saturate at a constant level, indicating that the observed linear dynamic range of the assay could be limited by the fluorescence detector used in the qPCR machine. To test such instrumental limitation, the same assays were conducted using a commercial microplate reader with high sensitivity and a broad dynamic range. Indeed, the microplate reader yielded dynamic signal response with no saturation (data not shown), enabling quantitative detection with a linear range extended up to 10 pM miR-21 (data not shown). These results verify the instrumental limitation of the qPCR machine on the dynamic range of this assay and suggest the feasibility of achieving quantitative miRNA detection across a >4-log concentration range (from ˜5 fM to 100 pM) via coupling this assay with a proper detector. Despite its limited dynamic range, the qPCR machine was used in the subsequent studies because it allows direct comparison between this new method and the standard qPCR assays on the same instrument. The analytical performance of the present assay was systematically validated by the parallel measurements with gold standard RT-qPCR. The EXTRA-CRISPR one-pot assay offers comparable detection sensitivity compared to a commercial miRCURY LNA miRNA PCR kit with which a LOD of 1.57 fM was obtained following the recommended two-step, 3-h protocol (data not shown).

To assess the specificity of the EXTRA-CRISPR method, the miR-21 assay was extended to detect a number of non-target miRNAs including a synthetic miR-21 (SEQ ID NO: 14) with a single-nucleotide mismatch at the ligation site for Padlock-1 (mirR-21 Mismatch-1, SEQ ID NO: 15) and eight human miRNAs at 1 pM each. These tests yielded a signal level of ˜2% of the miR-21 signal or even lower (FIG. 4K), demonstrating the excellent specificity of our assay. Such performance can be attributed to the multi-layered protection by the specific reactions for target-padlock hybridization, ligation⁶⁰, RCA, and Cas12a activation and cis-cleavage³¹. The effect of mismatch location on the assay specificity was then investigated. The discrimination power was observed to be highest for the single mismatches located at the ligation site and on either side of the ligation site and reduced progressively with the increasing distance from the ligation site, with mismatches tested at positions 3, 5, 7, and 11 nucleotides from the ligation site (data not shown), owing to decreasing impact of single mismatches on the padlock/target hybridization. Phi29 polymerase might also contribute to the non-specific amplification when the single mismatches approach the 3′ terminus because its 3′→5′ RNase activity can digest the terminal mismatched nucleotides by proof-reading to restart polymerization.⁶² A reasonably good specificity was still obtainable for a single mismatch shifted 3 nucleotides from the ligation point, with a non-specific signal level of ˜25%. These findings demonstrate that padlock design and optimization can affect single-base specificity. Moreover, the approach was adapted to detect a different target, miR-17, using four padlocks designed to differ in the 3′-end bases. Consistently, these single-mismatched padlocks yielded <2% non-specific signals with respect to the perfectly matched padlock (data not shown). Overall, these results validate the established one-pot EXTRA-CRISPR assay for rapid miRNA detection with single-digit fM sensitivity and single-base specificity using a properly engineered padlock probe.

Quantitative Profiling of EV miRNAs for Pancreatic Cancer Diagnosis

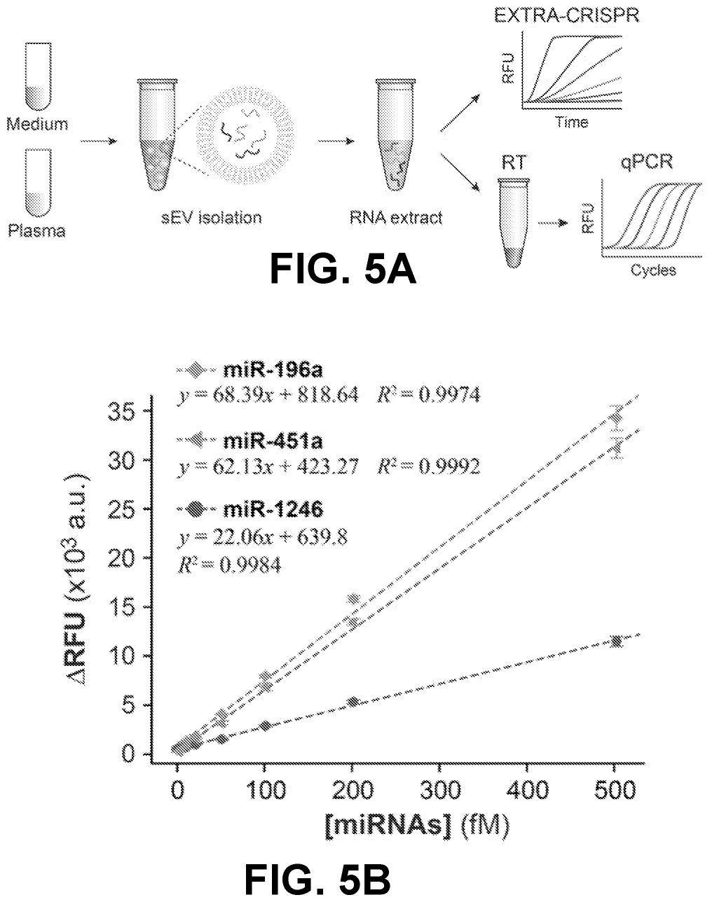

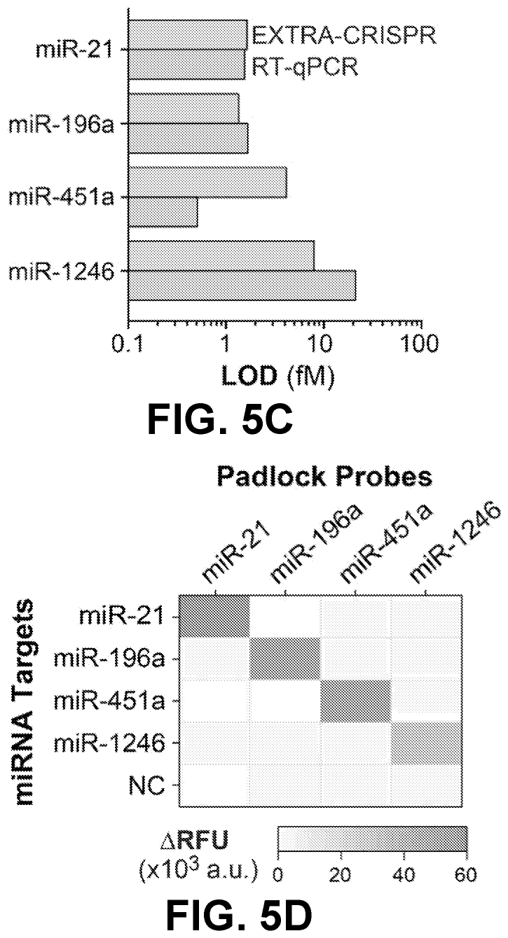

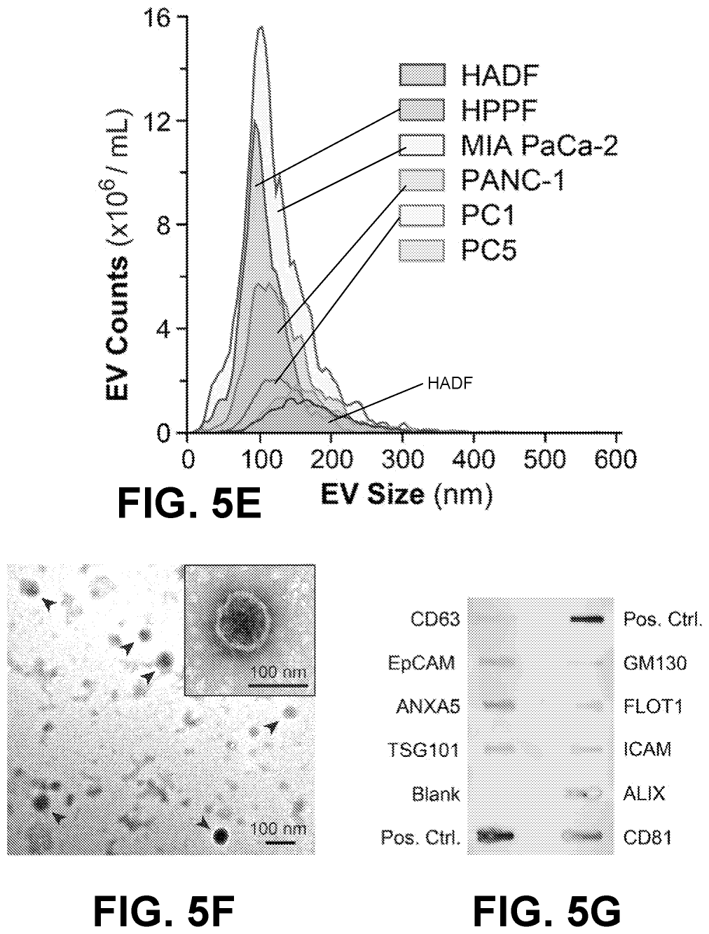

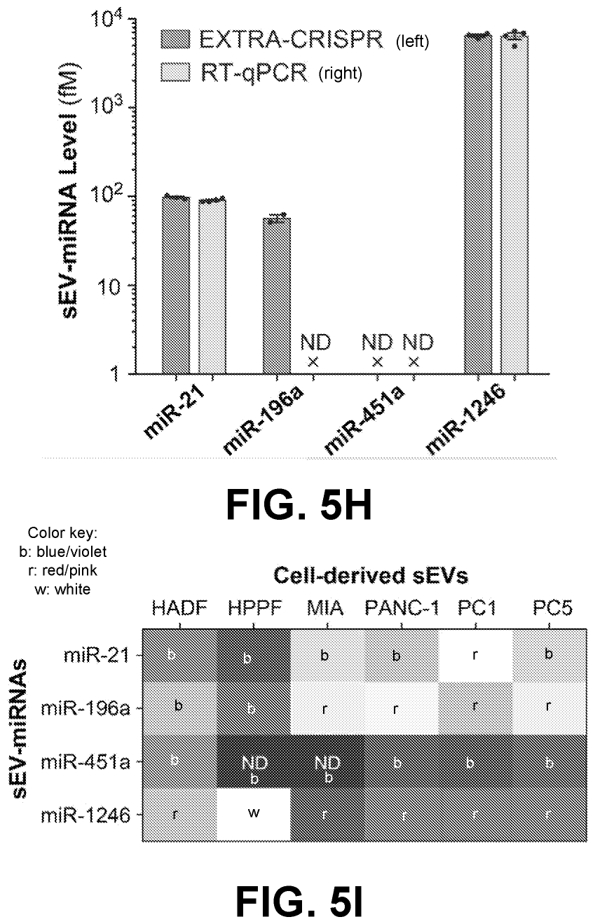

To demonstrate potential biomedical applications, the EXTRA-CRISPR assay was adapted to detect small EV miRNAs for diagnosis of PDAC. To this end, both cell culture media and human plasma samples were used to isolate small EVs (sEVs) and extract short RNAs from the sEV preparations, followed by parallel measurements with the one-pot EXTRA-CRISPR and two-step RT-PCR assays (FIG. 5A). Prior studies have identified numerous EV-miRNAs associated with human PDAC, from which four serum/plasma-derived EV miRNAs were selected that are frequently reported to be dysregulated in PDAC: miR-21,⁶³⁻⁶⁶ miR-196a,^{63, 65, 67-69} miR-451a,^{64, 70-72} and miR-1246.^{67, 73} As described above for miR-21, three EXTRA-CRISPR assays were established to detect miR-196a, miR-451a, and miR-1246 with a specific padlock probe, respectively (see Example 2 regarding padlock design and sequences). FIG. 5B presents the calibration plots for detecting the three miRNAs by EXTRA-CRISPR from which the LOD was determined to be 1.35 fM (5-500 fM linear range, R²=0.9974) for miR-196a, 4.14 fM (5-500 fM linear range, R²=0.9992) for miR-451a, and 7.96 fM (20-500 fM linear range, R²=0.9984) for miR-1246. Consistent with the case for miR-21, these LODs were comparable with those of the standard RT-qPCR assays which were calculated to be 1.69 fM miR-196a, 0.51 fM miR-451a, and 21.1 fM miR-1246 from the calibration curves with a threshold Ct value of 35 (FIG. 5C). These assays were also demonstrated to afford highly specific detection of the four miRNA targets with minimal cross-reactivity between individual padlock probes and three non-targets (FIG. 5D). Overall, these results corroborate the excellent adaptability of our EXTRA-CRISPR method to sensitive and specific detection of miRNAs with competitive performance to RT-qPCR.

The one-pot EXTRA-CRISPR assays were assessed using purified EVs from human adult dermal fibroblasts (HADF) and human primary pancreatic fibroblast (HPPF) as the normal controls, pancreatic ductal adenocarcinoma (PDAC) cell lines (MIA-PaCa-2 and PANC-1), and PDAC patient-derived xenograft (PDX) cell lines (PC1 and PC5). Small EVs were isolated from the conditioned media by ultracentrifugation (UC)⁷⁴ and characterized by nanoparticle tracking analysis (NTA) and transmission electron microscopy (TEM). The majority of isolated EVs displayed relatively small sizes within a range of ˜40-250 nm (FIG. 5E), which is typically observed for UC isolates,^{75, 76} and the morphological characteristics of sEVs (FIG. 5F).^{75, 76} Abundance of each cell line-derived sEVs was also measured by NTA to prepare standards for quantitative assessment of the one-pot miRNA assays. Using a commercial exosome antibody array, the quality of EV preparations was further verified by positive detection of several generic exosome protein markers (e.g., tetraspanins, Alix, ANXA5, and TSG101) and a weak signal for GM130, a control for cellular contamination (FIG. 5G).

Next, miRNA profiling of MIA-PaCa-2 (MIA)-derived sEVs was conducted using the established EXTRA-CRISPR and RT-qPCR assays in parallel (see Methods, below). As depicted in FIG. 5H, the one-pot assay yields high consistency with RT-qPCR in measuring the concentration of miR-21 (97.42 fM by one-pot vs. 90.13 fM by RT-qPCR) and miR-1246 (6.43 pM by one-pot vs. 6.35 pM by RT-qPCR) in the samples. However, miR-196a was only detected by our one-pot assay with a determined concentration of 56.7 fM. For RT-qPCR analysis, Ct values larger than 35 cycles for miR-196a in MIA-sEVs were experienced, which prevented precise quantification. This unexpected qPCR omission can be presumably attributed to that the poly(A)-tailing method may be not robust enough to reverse transcribe miR-196a in the intricate miRNA extract where unexpected secondary structures and hybridization may hinder reverse transcription.⁷⁷ In contrast, the EXTRA-CRIPR one-pot assay benefits from a denaturing and annealing step in sample preparation that eliminates potential secondary structures, empowering its robustness for detecting miRNAs in complicated biological samples. The level of miR-451a in MIA sEVs appeared to be too low to be quantifiable for both methods (FIG. 5H). The abundance of the targeted sEV-miRNAs for all six cell lines were measured by the one-pot assays and summarized in FIG. 5I. Compared to HADF and HPPF control cell lines, three miRNAs (miR-21, miR-196a, and miR-1246) were elevated in EVs from the PDAC-derived cell lines, while miR-451a was nondetectable in HPPF and MIA sEVs and very low (<˜0.02 copies/EV) in other four cell line-derived sEVs. Relatively high miR-196a and miR-1246 levels were observed in PDAC cell-derived sEVs, which is consistent with the previous study.⁶⁷ These findings on cell lines warrant further investigation of these sEV-miRNA markers using clinical human specimen for potential application to liquid biopsy-based diagnosis of PDAC.

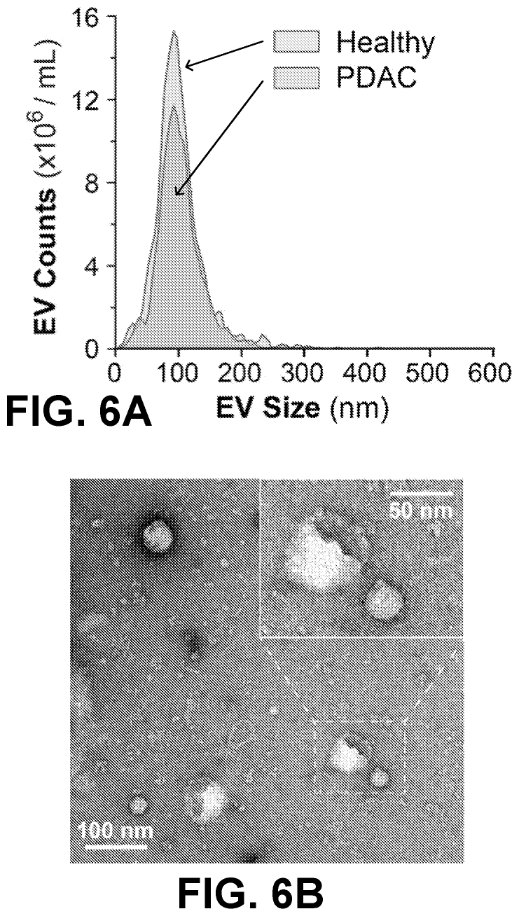

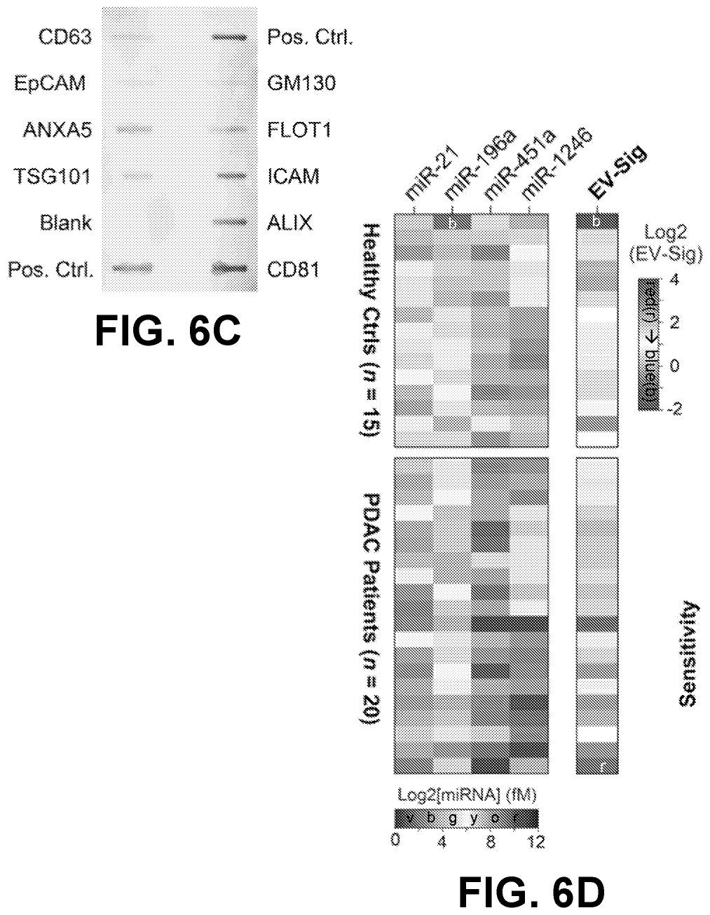

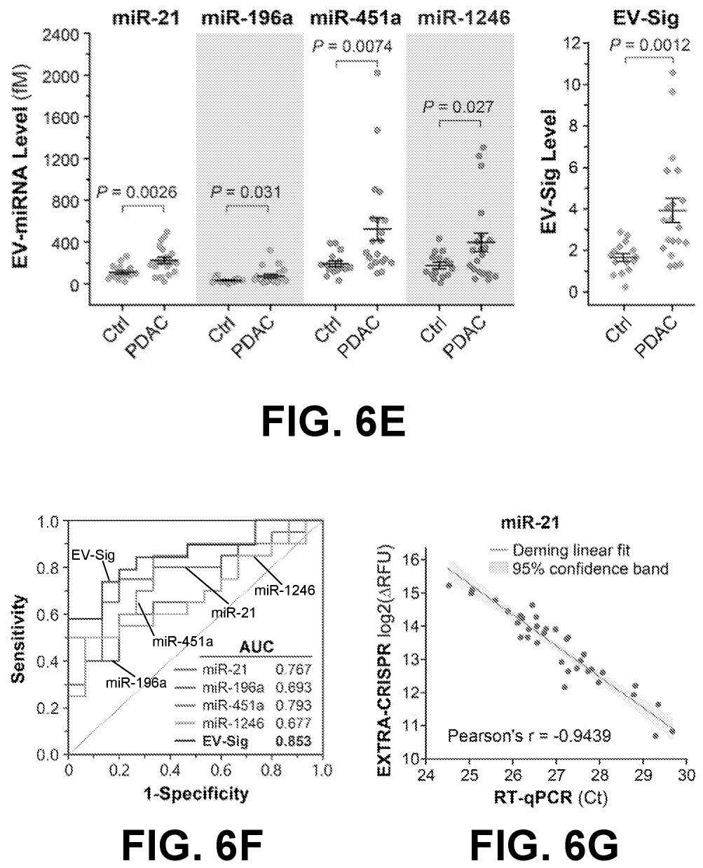

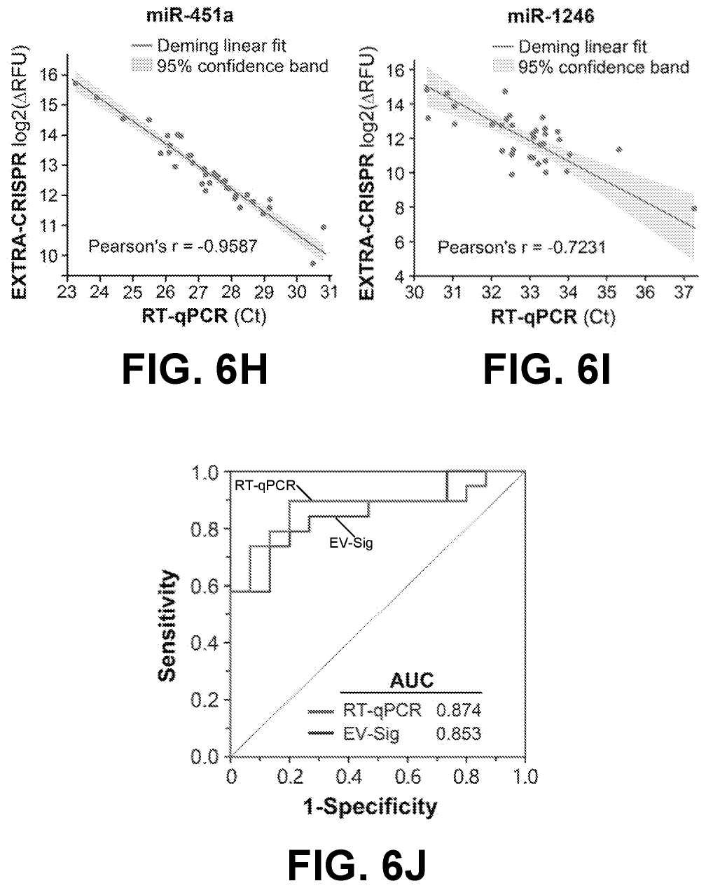

The EXTRA-CRISPR assay was then assessed for measuring clinical plasma samples from cancer-free controls (n=15) and patients with PDAC (n=20). As illustrated in FIG. 5A, the analysis workflow starts with isolating total circulating EVs from these plasma fluids (0.2 mL each) for subsequence small RNA extraction. To this end, a commercial PEG precipitation kit was used to afford faster and more efficient EV isolation than UC and hence to maximize the miRNA yield.^{78, 79} NTA analysis revealed the significant subject-to-subject heterogeneity in EV abundance varying from 6×10¹⁰ to 58×10¹⁰ EVs mL⁻¹ and in mean diameters of ˜98-143 nm with the major size ranges from ˜40 to 250 nm (FIG. 6A). The isolated plasma EVs were also checked with TEM, which observed the consistent vesicle sizes and the characteristic cup or round-shaped morphologies (FIG. 6B). Statistical comparison showed no significant difference between the control and patient groups in the NTA measured EV levels (P=0.79) and mean sizes (P=0.70), respectively (supplementary data not shown). The EV quality was further assessed with the exosome antibody array, which verified highly enriched exosomal vesicles against other cellular contaminations in these plasma EV preparations (FIG. 6C).

Next, EV miRNAs were extracted and measured with the EXTRA-CRISPR to quantify the levels of miR-21, miR-196a, miR-451a, and miR-1246 simultaneously. Although a spiking-in control is usually used for normalization of miRNA extraction and quantification, this approach remains arguable because of the lack of reliable reference miRNA in EVs, variations in extraction efficiency for reference RNAs versus miRNAs in biofluids, and different stability of spike-in miRNAs in blood compared to the endogenous ones.^{45, 80-83} Therefore, this assay was assessed for potential clinical analysis without using known miRNA and small RNA controls, such as U6 that was found unsuitable for qPCR quantification of circulating miRNAs.^{84, 85} To ensure the rigor of our analysis, the repeatability of our miRNA extraction protocol was tested based on a commercial kit using sEVs isolated from five human plasma samples. Based on the additional testing (supplementary data not shown) the miRNA extraction protocol was confirmed to confer excellent reproducibility for extraction of two different spike-in controls from EVs (0.4% CV for UniSp2 and 0.5% CV for UniSp4). Constant input volume of different plasma samples and considered the variation in EV abundance among different subjects was also used as a biological noise for disease diagnosis⁸². To mitigate batch-to-batch variation in our measurements, a synthetic copy of each miRNA marker was assayed as the positive control along with the clinical samples for data normalization. Lastly, the EXTRA-CRISPR results were rigorously validated by the standard RT-qPCR measurements of the same samples in parallel.

FIG. 6D summarizes the concentrations of the four individual plasma EV miRNA markers for each subject calculated from the calibration plots, showing their elevated expression in the PDAC cohort, in line with the previous studies.^{67, 70, 86}. It is noted that compared to other three EV-miRNAs, miR-196a showed lower abundance in most of the tested clinical samples and less difference between the healthy and PDAC groups. Consistently, machine learning analysis of the data using the least absolute shrinkage and selection operator (Lasso) regression eliminated miR-196a and defined an EV signature (EV-Sig) of each subject as the weighted linear combination of miR-21, miR-451a, and miR-1246 (FIG. 6D, and see Methods below). As plotted in FIG. 6E, the EV-Sig improves the ability to differentiate the PDAC group against the healthy group (P=0.0012, two-tailed Student's t-test with Welch correction), compared to the individual markers (miR-21, P=0.0026; miR-196a, P=0.031; miR-451a, P=0.0074; and miR-1246, P=0.027). The diagnostic performance of these EV-miRNAs and EV-Sig was quantitatively evaluated by receiver operating characteristic (ROC) curve analysis (FIG. 6F). Single-marker detection yielded the modest area under the curve (AUC) values ranging from 0.677 [95% Confidence Interval (CI), 0.486-0.868] for miR-196a to 0.793 (95% CI, 0.635-0.951) for miR-451a. The EV-Sig panel combining miR-21, miR-451a, and miR-1246 greatly improves the diagnostic power to an AUC of 0.853 (95% CI, 0.726-0.979).

To validate the EXTRA-CRISPR measurements, the same RNA extracts were analyzed by the established RT-qPCR assays in parallel to quantify the four EV-miRNA markers (Supplementary data not shown). Strong correlation was observed between the two methods for detecting miR-21 and miR-451a, as indicated by the Pearson's r of −0.9439 and −0.9587, respectively (FIG. 6G, 6H). The correlation obtained for miR-1246 was modest (Pearson's r=−0.7231), which can be attributed to the ultralow levels of miR-1246 in most of the samples (Ct>32) that can lead to variations in analysis (FIG. 6I). Consistent with the cell line results (FIG. 5H), the poly(A)-tailing RT-PCR assay was unable to detect EV-miR-196a in 40 thermal cycles for almost all the clinical samples tested, while the EXTRA-CRISPR assay successfully detected the low-abundance EV-miR-196a in these samples. Thus, the RT-qPCR results of miR-21, miR-451a, and miR-1246 were assessed by the ROC analysis, yielding the AUC values consistent with that of one-pot assays (Supplementary data not shown). The RT-qPCR signature combining three markers obtained through Lasso regression confers an AUC of 0.874 (95% CI, 0.757-0.991) for PDAC detection. This suggests that the EXTRA-CRISPR assay confers a competitive performance with the RT-qPCR counterpart for plasma EV-based diagnostics targeting the same three-miRNA marker panel (FIG. 6J). Overall, these comparative assessments with clinical samples further support the high sensitivity, specificity, and robustness of our method for miRNA analysis towards potential applications for liquid biopsy-based cancer diagnosis.

### EXTRA-CRISPR Assay for Point-of-Care (POC) Testing

The simplicity of the isothermal one-pot EXTRA-CRISPR miRNA assay makes it intrinsically adaptable to point-of-care (POC) applications where minimal needs for instrument is desired. To test such feasibility, a low-cost, portable smartphone-based EXTRA-CRISPR assay system was built. This prototype was assembled from the 3D-printed body parts, a blue LED illuminator, a plastic filter (520 nm long-pass), and a consumer digital hotplate (coffee mug warmer) (FIG. 7A). A smartphone was mounted on the device to acquire fluorescence images which were then analyzed to measure the fluorescence intensity. The POC prototype was first assessed for the isothermal EXTRA-CRISPR reaction in comparison to the commercial qPCR thermocycler. In this case, the reaction tubes were simply laid on the flat hotplate surface inside the POC device which was kept at 37° C. to conduct the reaction for 2 hours. This simple heating method was found to preserve ˜84% of the amplification efficiency obtained with the sophisticated PCR thermocycler (FIG. 7B, left), indicating the robustness of EXTRA-CRISPR assay that negates sophisticated heater design for precise temperature control. The smartphone-based POC device was then tested for real-time fluorescence detection of the EXTRA-CRISPR reactions. The observed detection curves were consistent with those of the RT-qPCR instrument (FIG. 7B, right), demonstrating the good performance of the prototype for quantitative miRNA detection. The POC device was further calibrated via targeting three miRNA markers selected above for pancreatic cancer detection, as presented in FIGS. 7C-7E. Compared to the EXTRA-CRISPR detection using the qPCR machine, coupling the assay with the simple low-cost device afforded slightly lower sensitivity and comparable dynamic range for miR-21 (LOD: 6.10 fM, linear range: 20-500 fM), miR-451a (9.64 fM, 10-500 fM), and miR-1246 (9.92 fM, 50-1000 fM), respectively. Lastly, as a proof-of-concept for clinical applications, the POC device was assessed for ability to detect these markers in sEVs isolated from the plasma samples of four control donors and four PDAC patients. As seen in FIGS. 7F and 7G, the POC device was able to detect the differential levels of three sEV-miRNA markers to discriminate the PDAC group versus the control group, which is consistent with the diagnostic results of the isothermal assays conducted on the PCR machine (FIG. 6E).

In addition to the smartphone-based fluorescence detector, the adaptability of this assay to instrument-free POC diagnostics was also investigated. To this end, an EXTRA-CRISPR test coupled with lateral flow assay (EXTRA-CRISPR-LFA) was developed using a commercially available LFA strip for visual detection.⁸⁷ In this test, as illustrated in FIG. 7H, the ssDNA Cas12a reporter was tagged with a biotin and a carboxyfluorescein (FAM) dye molecule at each end so that it can be captured on the pre-loaded anti-FAM antibody coated gold nanoparticles when flowing through the LFA strip. The complexes formed with intact reporters will be retained on the streptavidin band via their biotin tags, producing a positive control line. If the immuno-gold particles bind with cleaved reporters, they can flow downstream to be captured by the secondary antibody deposited on the test line that specifically recognizes the anti-FAM antibody. FIG. 7I presents the results for miRNA detection using the standards of three miRNAs (see Methods for details), which shows increasing intensity of the test line with the target concentration at the low levels. The test line intensity was seen to decrease at 100 pM miR-21, in consistence with the amplification suppression observed in FIGS. 2B-2C. The signals for miR-451a and miR-1246 saturated at the relatively high concentrations, which can be attributed to the limited total amount of immuno-gold nanoparticles, as indicated by the diminished intensity of corresponding control lines. Compared to the fluorescence detection modality (FIGS. 7A-7G), visual LFA detection exhibited reduced performance for quantitative analysis, as manifested by the decreased linearity of the calibration plots, sub-100 fM LODs, and <2-log dynamic ranges (supplementary data not shown). Nonetheless, it is noted that the EXTRA-CRISPR-LFA test greatly outperforms the existing LFA methods for miRNA detection with pM-level LODs.⁸⁸

Using the same patient samples for testing the smartphone device, the applicability of the EXTRA-CRISPR-LFA test for instrument-free clinical analysis of sEV miRNAs was demonstrated. Among the three markers, LFA detection of sEV miR-451a yielded the highest visual signals (FIG. 7J) and the best diagnostic performance (FIG. 7K) to detect the PDAC samples, which is consistent with the results of smartphone detection (FIG. 7G). Further quantitative comparison shows a strong linear correlation between the two POC methods for detecting miR-451a (Pearson's r=0.949, FIG. 7L) and miR-21 (Pearson's r=0.942, FIG. 7M). Such correlation became weaker for miR-1246 with the lowest levels detected in PDAC patient-derived sEVs (Pearson's r=0.527, Supplementary FIG. 7N), due to the relatively limited sensitivity and quantitative performance of LFA. Nonetheless, these results verify the ability of the LFA assay for instrument-free semi-quantitative miRNA detection with competitive sensitivity. Overall, these on two commonly adapted POC detection modalities demonstrate the potential of the simple EXTRA-CRISPR technology of the present disclosure as an adaptable platform to develop new low-cost POC diagnostic tests.

### DISCUSSION

Among many isothermal methods developed for miRNA sensing, RCA is a proven technique with the advantages in simplicity, specificity, and robustness. To enhance the sensitivity limited by the linear amplification, several approaches have been explored to exponentiate RCA, including the target-primed branched RCA using a second primer⁸⁹ and the nicking endonuclease-assisted exponential RCA.^{13, 90} compared to these methods, CRISPR-Cas systems emerge as a compelling platform for miRNA detection owing to their substantial potential to promote both detection sensitivity and specificity.^{30, 31} Nonetheless, like other CRISPR-based nucleic acid tests reported, the prevailing strategy used in the existing methods is tandem hyphenation of two independent reactions for miRNA pre-amplification and amplicon detection by Cas12a trans-cleavage (Table 1). A challenge in combining RCA (and other amplification assays) and CRISPR assays may be attributed to the double-edged effects of CRISPR-Cas12a, as revealed by the above mechanistic studies (FIGS. 2A-2H), where indiscriminate trans-cleavage of Cas12a causes degradation of the essential ssDNA reactants (i.e., padlock probe and secondary targets) to suppress the exponential amplification. To overcome this issue, a strategy was developed that combines engineering of the CRISPR-reactive padlock probe and balancing the reaction kinetics and equilibria to promote the desired Cas12a functions in a complex tri-enzymatic reaction network (FIG. 2A and FIGS. 4A-4K). This novel strategy allowed establishment of the first one-step, one-pot isothermal assay that collaboratively couples RCA with the CRISPR-Cas12a system to enact exponential amplification and fluorogenic detection of miRNAs. Moreover, the work in this example describes the feasibility of harnessing both cis- and trans-activities of CRISPR-Cas systems for biosensing, paving a new way for developing the next-generation CRISPR diagnostics.

High sensitivity improves detecting miRNA signatures of tumors in biospecimens. The concentrations of miRNA markers in biofluids can be at the picomolar level or even lower, especially at the early disease stages.⁹¹ Relevant to this work, the EV population is considered as a major carrier of miRNAs in human biofluids and exosomes secreted by tumor cells offer a promising route to explore disease-specific miRNA signatures.⁹²⁻⁹⁴ However, it has been shown that the averaged load of a miRNA target in EVs can be as low as 10⁻⁵ copies per vesicle,^{13, 95} which makes sensitive miRNA analysis essential to clinical biomarker development. Embodiments of the EXTRA-CRISPR miRNA assay of the present disclosure offer single-digit fM sensitivity and single-nucleotide specificity, which is comparable with RT-qPCR,⁹⁶ while greatly simplifying and expediting the analysis workflow. The assay of the present disclosure is also cost-effective with the material cost estimated to be as low as $0.60 per test at the research scale (see Table 1). Such combination of sensitive and specific analytical performance, one-pot contamination-proof operation, and low cost presents the method of the present disclosure as a radical improvement to the existing CRISPR-based miRNA assays.^{34, 36, 38, 97} and a competitive alternative to other existing miRNA analysis technologies, such as ddPCR,⁹⁸ microarrays,⁹⁹ and nanopore biosensors⁵².

PDAC that accounts for ˜90% of all pancreatic neoplasms is an extremely aggressive and lethal gastrointestinal malignancy that is projected to be the second-leading cause of cancer-related mortality by 2030, with an overall 5-year survival rate of 11% and an incidence increase rate of 0.5%-1.0% per year.¹⁰⁰⁻¹⁰² Due to its asymptomatic course, rapid progression, and delayed clinical presentation, PDAC is considered a silent killer, and most patients were diagnosed at an advanced stage.^{103, 104} Early detection of PDAC at resectable stages has profound impact on changing the malignancy's poor survival figures.^{105, 106} However, no reliable screening method, either molecular or imaging-based, exists to date to allow accurate early detection of asymptomatic PDAC patients. Serum carbohydrate antigen 19-9 (Ca19-9) detection is the most extensively adapted marker for PDAC diagnosis which was approved by the U.S. Food and Drug Administration (FDA) in 2002. Ca19-9 can help with PDAC prognosis and monitoring, however, it is not recommended as a biomarker for early detection due to its low positive predictive value (0.5-0.9%).¹⁰⁷

Increasing evidence has indicated the promising potential of circulating EVs as a rapid, minimally invasive, efficient, and cost-effective liquid biopsy in developing diagnostic biomarkers of PDAC.⁸⁶ Using PDAC as the disease model, the feasibility of the EXTRA-CRISPR assay was assessed for clinical analysis of miRNA markers in circulating EVs for PDAC diagnosis. The assay demonstrated good compatibility with two commonly used EV isolation methods. Targeting four PDAC-related markers (miR-21, miR-196a, miR-451a, and miR-1246), highly sensitive and specific miRNA profiling of EVs derived from cell lines and clinical plasma specimens was demonstrated. While individual EV-miRNA tests only yielded modest diagnostic power, an EV signature (EV-Sig) can be defined from the four-marker panel by machine learning analysis to improve the PDAC detection with an AUC of 0.853 (FIG. 6F). This diagnostic performance is comparable with that of the serum Ca19-9 test previously reported,¹⁰⁸ which supports the clinical potential of EV-miRNA markers for PDAC diagnosis. Rigorous validation by RT-qPCR analysis of the same samples was conducted in parallel to the one-pot assays and showed strong correlation in both analytical and diagnostic performance between the two methods, confirming the robustness of the EXTRA-CRSPR assay. It is noted that a goal of this clinical study was to assess a new bioassay rather than the biomarkers. To this end, the panel of four miRNAs highly associated with PDAC were gathered from literature. An improved EV-Sig, constituted with an optimal miRNA biomarker panel for PDAC diagnosis, could enhance the diagnostic power of our new one-pot assays. Future work of large-scale screening and validation of circulating EV-miRNAs using the developed EXTRA-CRSPR assay could facilitate the identification of potent EV-miRNA biomarker candidates in PDAC, and beyond.

Overall, featuring a combination of sensitive and specific analytical performance, easy operation, rapid reaction, and low cost, the EXTRA-CRISPR provides a competitive alternative to standard RT-qPCR for miRNA analysis in biological and clinical samples. The simplicity of the method would greatly facilitate future instrumentation or microfluidic miniaturization and integration to fully automate the workflow, further increase analysis throughput and reproducibility, and reduce sample consumption and turnaround time. For instance, this assay can be readily integrated with microfluidic EV isolation^{109, 110} to greatly streamline the analytical pipeline and improve the performance for clinical analysis of EV miRNA markers. In addition to biosensing development and instrumentation, another promising application area of the one-pot EXTRA-CRISPR assay described herein would be POC diagnostics where cost-effective, portable devices are needed for rapid detection of infectious pathogens in resource-limited settings. The proof-of-concept studies using both smartphone- and LFA-based modalities demonstrate the ability of the EXTRA-CRISPR technology to afford improved analytical performance for miRNA detection compared to the existing POC methods.⁸⁸ These results support the feasibility of our EXTRA-CRISPR technology to open new opportunities for developing low-cost, yet sensitive nucleic acid tests for broad POC applications, such as SARS-CoV-2 diagnosis.^{111, 112} The described CRISPR-enabled isothermal amplification technology could provide a versatile platform for developing new biosensors for other types of biomarkers such as proteins and small molecules, using aptamers or other nucleic acids as a bridge.^{113, 114} Therefore, this method provides a useful tool to facilitate the development of next-generation biosensing technologies and new clinical biomarkers. Therefore, this new approach can provide immense opportunities for the next-generation CRISPR diagnostics to address the needs in broad areas, including biological research, clinical lab diagnosis and POC testing.

### Methods

Materials. RNA and DNA oligos were purchased from IDT (Integrated DNA Technologies), SplintR buffer, dNTP (10 mM), BSA (20 mg/mL), phi29 polymerase (10 U/μL), SplintR ligase (25 U/μL), T4 DNA ligase (400 U/μL), cas12a (1 μM), and ATP are purchased from NEB (New England Biolabs Inc). SYBR Green II dye, SYBR gold nucleic acid gel stain, agarose, and 10×TBE buffer were purchased from Thermo Fisher Scientific. SeraMir™ exosome RNA amplification kit (RA806A-1), SeraMir exosome RNA column purification kit (RA808A-1), and Exo-Check™ exosome antibody array (EXORAY200B-4) were purchased from SBI (System Biosciences). miRCURY LNA™ RT kit, miRCURY LNA™ SYBR Green PCR kit, miRNA specific primers and the spike-in control were purchased from Qiagen. Pierce™ rapid gold BCA protein assay kit (A53226) was purchased from ThermoFisher Scientific. WesternBright™ SiriUS™ chemiluminescent HRP substrate was purchased from advansta.

Procedure of EXTRA-CRISPR assay. For the preparation of RNP, 2 μL of water, 0.5 μL of 2.1 buffer (NEB), 1 μL of cas12a (1 μM) and 1.5 μL of crRNA (1 μM) was incubated together at 37° C. for 30 min. The usage of each component can be adjusted according to the experimental consumption. For a 20-μL reaction, padlock (2 μL) and miRNA (2 μL) in buffer (0.4 μL) are first denatured at 80° C. for 5 min and cooled down to room temperature in about one minute, and then added to the reaction system containing other components (15.6 μL in total, with 0.8 μL of dNTP, 0.2 μL of BSA, phi29 polymerase, SplintR ligase, RNP, reporter DNA, 1.6 μL of 10× buffer and water). After mixing, the assay was performed in a qPCR device (Bio-Rad, CFX connect real-time system) at 37° C. for a certain period, and the fluorescence intensity is monitored every 1 minute. The final concentration of miRNA in the 20-μL reaction system was used to determine the analytical sensitivity.

Procedures of RCA only, two-step method and three-step method. In the RCA only method, padlock (1 μL), miRNA (1 μL), and SplintR buffer (0.2 μL) were first denatured at 80° C. for 5 min and cooled down gradually to room temperature before being added to the ligation system which contains 0.8 μL of SplintR buffer, 0.25 μL of SplintR ligase and 6.75 μL of water. Ligation was performed at 37° C. for 2 h and then 65° C. for 10 min. Then 2 μL of the ligation product was mixed with the 20-μL RCA reaction system which contains 2 μL of phi29 buffer, 0.4 μL of phi29, 0.2 μL of BSA, 0.8 μL of dNTP, 2 μL of 10×SYBR Green II, and 12.6 μL of water. RCA was performed at 37° C. for 2 h in the qPCR device and monitored every 1 minute. The miRNA concentration in the ligation system was used to determine the analytical performance. In the two-step method, 2 μL of padlock, 2 μL of miRNA and 0.4 μL of SplintR buffer were first denatured and annealed and then transferred to the 20-μL ligation-RCA system which contains 1.6 μL of SplintR buffer, 0.8 μL of dNTP, 0.2 μL of BSA, 0.2 μL of phi29, 0.5 μL of SplintR and 12.3 μL of water. The reaction was conducted at 37° C. for 2 h and then 65° C. for 10 min, next, 5 μL of RNP and 0.2 μL of the reporter (100 μM) were added to the system and incubated at 37° C. for 2 h in the qPCR device. The miRNA concentration in the ligation-RCA system was used to determine the analytical performance. In the three-step method, ligation and RCA step were the same with the RCA only method, after RCA, the enzyme was inactivated at 65° C. for 10 min followed by the addition of 5 μL of RNP and 0.2 μL of the reporter (100 μM), the final step was carried out at 37° C. for 2 h in the qPCR device. The miRNA concentration in the ligation system was used to determine the analytical performance.

Agarose gel electrophoresis. The gel electrophoresis experiments were performed using the 3% agarose gel in TBE buffer at 110 V for 1 h. After that, the gel was immersed into the TBE buffer containing SYBR gold dye for 30 min. The image was captured with an imager (Odyssey, LI-COR).

RT-qPCR detection of miRNAs. The 10-μL of the reverse transcription contains 2 μL of 5×miRCURY RT reaction buffer, 1 μL of 10×miRCURY RT enzyme mix, 1 μL of miRNA, and 6 μL of water. The RT system was incubated at 42° C. for 60 min and then inactivated at 95° C. for 5 min. The cDNA solution was diluted by 60× before being added to the qPCR system which contains 5 μL of 2×miRCURY SYBR green master mix, 1 μL of the PCR primer, 3 μL of cDNA template, and 1 μL of water. Temperature program: 95° C. for 2 min, 40 cycles of 95° C. for 10 s, and 56° C. for 60 s.

Cell culture. Procedures for cell culture and the harvest of EV-contained conditioned SFM (serum-free medium) were as previously described.^{115, 116} Briefly, immortalized PDAC cell lines (MIA-PaCa-2 and PANC-1), primary human xenograft-isolated PDAC cell lines (PC1, PC5), as well as human fibroblast cell lines (HADF and HPPF) were cultured in DMEM-F12 supplemented with 10% FBS and 1% antibiotic antimycotic solution. Culture medium was replaced by DMEM-SFM when ˜85% confluence was reached. Conditioned-SFM were collected after 48 h and stored at −80° C. until analysis.

EV isolation from cell culture medium. All the centrifugations were performed at 4° C. with the following procedure: 300 g for 10 min to remove cells, 2,000 g for 20 min to remove dead cells and cell devirs, 10,000 g for 30 min to remove large EV particles (e.g., microvesicles), and 100,000 g for 2 h to collect the sEV pellet. The sEVs were resuspended in 100 μL of PBS for the following NTA analysis and miRNA extraction using SeraMir exosome RNA column purification kit.

EV isolation, miRNA extraction and detection from plasma samples. Human plasma was obtained from Clinical and Translational Science Institute, University of Florida (IRB202200150). Small EVs were isolated according to the protocol of the SeraMir™ Exosome RNA Amplification kit. First, thaw the plasma samples on ice and centrifuge at 3000 g for 15 min to remove cells and cell debris, followed by the centrifugation at 18000 g for 30 min to remove large vesicles. All the centrifugations were performed at 4° C. Then, 200 μL of the plasma was diluted to 250 μL with PBS before adding 60 μL of ExoQuick and incubated at 4° C. for 30 min. After incubation, the plasma was centrifuged at 13000 rpm for 2 min to collect the pellet. EV miRNA was purified and eluted in 30 μL of water according to the manufacturer's protocol for the following analysis. For the detection of miR-21, miR-196a, and miR-1246, 2 μL of the miRNA extract were added to the one-pot assay. As the expression level of miR-451a is extremely high, the extract was diluted 10 times before being added to the one-pot assay for the precise quantitation of miR-451. The signals using one-pot assay were subtracted by the corresponding background and normalized by miRNA positive controls. miRNA concentrations were tested by RT-qPCR simultaneously, the extract of was also diluted 10 times before being added to the reverse transcription system when detecting miR-451a. The cDNA solution was diluted 30× to perform the qPCR reaction.

Transmission electronic microscopy (TEM). Negative staining method is used for EV imaging. EVs were fixed in 2% paraformaldehyde (PFA) for 5 min and loaded on 200 mesh copper grids. TEM was performed at the Electron Microscopy Core of University of Florida on a Hitachi 7600 transmission electron microscope (Hitachi High-Technologies America, Schaumburg, IL) equipped with a MacroFire® monochrome progressive scan CCD camera (Optronics, Goleta, CA).

NTA analysis. Particle number and size distribution of EVs isolated from medium and plasma samples were determined by nanoparticle tracking analysis (NTA) using a ZetaView system (Particle Metrix Inc.). Samples were diluted in PBS to an acceptable concentration, according to the manufacturer's recommendations.

Antibody arrays for the detection of exosomal biomarkers. The protein biomarkers of exosomes isolated from the PDAC cell line and PDAC patient's plasma were tested using the Exo-Check™ exosome antibody array. Isolated EVs were resuspended in PBS and the amount of protein in EV samples was determined by Pierce™ rapid gold BCA protein assay kit. 50 μg protein was used for the antibody array according to the manufacture's protocol. Briefly, the sample was lysed by lysis buffer and 1 μL of labeling reagent was added to the lysate followed by the incubation at room temperature for 30 min with constant mixing. After removing excess labeling reagent, lysates were mixed with 5 mL of blocking buffer. The blocking buffer/labeled exosomes lysate mixture was then incubated with the antibody precoated membrane at 4° C. overnight on a shaker. Next day, the membrane was washed carefully by wash buffer and incubated with 5 mL of detection buffer at room temperature for 30 min. After removal of the detection buffer and washing, the membrane was developed using the WesternBright™ SiriUS™ chemiluminescent HRP substrate. The image was captured using the LI-COR imager (Odyssey) with exposure time of 1 min.

Statistical analysis. Mean, standard deviation, and standard error were calculated with standard formulas in Excel. To compare the patient and control groups, a two-tailed Student's t-test with Welch correction was performed with a significance level of P<0.05. ROC analyses were performed to determine the AUC values using the OriginPro software (OriginLab Corporation). Machine learning analysis of the miRNA markers was conducted by fitting the data with the least absolute shrinkage and selection operator (Lasso) paths for regularized logistic regression,¹¹⁷ in which with the tuning parameter (I) selected by the Leave-one-out cross-validation.¹¹⁸ The estimated coefficients obtained for the markers by Lasso regression were used to create a weighted linear combination of the markers. Lasso regression was performed using the JMP Pro software (JMP Statistical Discovery LLC). A 95% confidence level was used for all statistical analyses.

EXTRA-CRISPR POC device. A coffee mug warmer (the digital hotplate) and LED illuminator were purchased from Amazon and Boli Optics, respectively, and other parts were designed using AutoCAD 2022 and fabricated using 3D printing (FormLabs Form 3+ SLA 3D printer). The device was assembled as illustrated in FIG. 7A. To conduct EXTRA-CRISPR reactions in the device, 15 μL of mineral oil was added to avoid evaporation, and the tubes were placed in the middle of the hotplate set to the actual temperature at 37° C. to initiate the reactions. Fluorescent photos were captured using a smartphone in a darkroom, and the exposure time was 1 s. The gray value of each reaction was analyzed using ImageJ software: First, split the color channels and keep the green channel of the image, then use the oval tool to determine the mean gray value in a certain area.

Lateral flow assay. The LFA strips were purchased from Milenia Biotec (HybriDetect-Universal Lateral Flow Assay Kit). The quenchers modified on reporters are replaced by biotin molecules (Biotin-TTATT-FAM). For the detection of miR-451a, the reporter concentration was decreased to 1 μM to avoid strong background signals, other reagents remain unchanged. The EXTRA-CRISPR reactions (20 μL) for LFA were first incubated in the PCR instrument and then mixed with 80 μL of HybriDetect assay buffer. After mixing, the HybriDetect dipstick was immersed into the solution with 5 min incubation time. The sticks were then taken out and photos were captured using iPhone XS. The LFA images were analyzed using the ImageJ software (NIH): first, convert images into the 8-bit type and subtract background; second, set measurements to analyze the integrated density and invert the image color; finally, use the rectangle tool to determine the intensity of the test band. In order to mitigate the variations from photographing, the LFA strips for the calibration and clinical studies were grouped together respectively to take the photos. Moreover, the LFA test line signals for miR-21, miR-451a, and miR-1246 were normalized with the averaged control line intensity measured for the negative controls, respectively, assuming the constant amount of pre-loaded gold nanoparticles and uniform capture efficiency of the control lines across LFA strips.

### REFERENCES

1. Ha, M. & Kim, V. N. Regulation of microRNA biogenesis. Nat. Rev. Mol. Cell Biol. 15, 509-524 (2014).
2. Di Leva, G., Garofalo, M. & Croce, C. M. MicroRNAs in cancer. Annu. Rev. Pathol. 9, 287-314 (2014).
3. Inui, M., Martello, G. & Piccolo, S. MicroRNA control of signal transduction. Nat. Rev. Mol. Cell Biol. 11, 252-263 (2010).
4. Lin, S. & Gregory, R. I. MicroRNA biogenesis pathways in cancer. Nat. Rev. Cancer 15, 321-333 (2015).
5. Mori, M. A., Ludwig, R. G., Garcia-Martin, R., Brandao, B. B. & Kahn, C. R. Extracellular miRNAs: from biomarkers to mediators of physiology and disease. Cell Metab. 30, 656-673 (2019).
6. Jet, T., Gines, G., Rondelez, Y. & Taly, V. Advances in multiplexed techniques for the detection and quantification of microRNAs. Chem. Soc. Rev. 50, 4141-4161 (2021).
7. Dave, V. P. et al. MicroRNA amplification and detection technologies: opportunities and challenges for point of care diagnostics. Lab. Invest. 99, 452-469 (2019).
8. Kilic, T., Erdem, A., Ozsoz, M. & Carrara, S. microRNA biosensors: Opportunities and challenges among conventional and commercially available techniques. Biosens. Bioelectron. 99, 525-546 (2018).
9. Ouyang, T., Liu, Z., Han, Z. & Ge, Q. MicroRNA detection specificity: recent advances and future perspective. Anal. Chem. 91, 3179-3186 (2019).
10. Forero, D. A., Gonzalez-Giraldo, Y., Castro-Vega, L. J. & Barreto, G. E. qPCR-based methods for expression analysis of miRNAs. Biotechniques 67, 192-199 (2019).
11. Hunt, E. A., Broyles, D., Head, T. & Deo, S. K. MicroRNA detection: current technology and research strategies. Annu. Rev. Anal. Chem. 8, 217-237 (2015).
12. Deng, R., Zhang, K. & Li, J. Isothermal amplification for microRNA detection: from the test tube to the cell. Accounts Chem. Res. 50, 1059-1068 (2017).
13. Cao, H., Zhou, X. & Zeng, Y. Microfluidic Exponential Rolling Circle Amplification for Sensitive microRNA Detection Directly from Biological Samples. Sensors and actuators. B, Chemical 279, 447-457 (2019).
14. Yan, H., Xu, Y., Lu, Y. & Xing, W. Reduced graphene oxide-based solid-phase extraction for the enrichment and detection of microRNA. Anal. Chem. 89, 10137-10140 (2017).
15. Deng, R. et al. Toehold-initiated rolling circle amplification for visualizing individual microRNAs in situ in single cells. Angew. Chem.—Int. Edit. 53, 2389-2393 (2014).
16. Qian, J., Zhang, Q., Liu, M., Wang, Y. & Lu, M. A portable system for isothermal amplification and detection of exosomal microRNAs. Biosens. Bioelectron. 196, 113707 (2021).
17. Jia, H. X., Li, Z. P., Liu, C. H. & Cheng, Y. Q. Ultrasensitive detection of microRNAs by exponential isothermal amplification. Angew. Chem.—Int. Edit. 49, 5498-5501 (2010).
18. Li, C., Li, Z., Jia, H. & Yan, J. One-step ultrasensitive detection of microRNAs with loop-mediated isothermal amplification (LAMP). Chem. Commun. 47, 2595-2597 (2011).
19. Park, C. R., Park, S. J., Lee, W. G. & Hwang, B. H. Biosensors using hybridization chain reaction—design and signal amplification strategies of hybridization chain reaction. Biotechnol. Bioprocess Eng. 23, 355-370 (2018).
20. Choi, H. M. et al. Programmable in situ amplification for multiplexed imaging of mRNA expression. Nat. Biotechnol. 28, 1208-1212 (2010).
21. Wu, H. et al. Label-free and enzyme-free colorimetric detection of microRNA by catalyzed hairpin assembly coupled with hybridization chain reaction. Biosens. Bioelectron. 81, 303-308 (2016).
22. Cheglakov, Z., Cronin, T. M., He, C. & Weizmann, Y. Live cell microRNA imaging using cascade hybridization reaction. J. Am. Chem. Soc. 137, 6116-6119 (2015).
23. Aita, A. et al. Serum miRNA Profiling for Early PDAC Diagnosis and Prognosis: A Retrospective Study. Biomedicines 9 (2021).
24. Shukla, N., Yan, I. K. & Patel, T. Multiplexed Detection and Quantitation of Extracellular Vesicle RNA Expression Using NanoString. Methods Mol. Biol. 1740, 177-185 (2018).
25. Srinivasan, S., Duval, M. X., Kaimal, V., Cuff, C. & Clarke, S. H. Assessment of methods for serum extracellular vesicle small RNA sequencing to support biomarker development. J. Extracell. Vesicles 8, 1684425 (2019).
26. Guerau-de-Arellano, M., Alder, H., Ozer, H. G., Lovett-Racke, A. & Racke, M. K. miRNA profiling for biomarker discovery in multiple sclerosis: from microarray to deep sequencing. J. Neuroimmunol. 248, 32-39 (2012).
27. Hong, L. Z. et al. Systematic evaluation of multiple qPCR platforms, NanoString and miRNA-Seq for microRNA biomarker discovery in human biofluids. Sci. Rep. 11, 4435 (2021).
28. Kaminski, M. M., Abudayyeh, O. O., Gootenberg, J. S., Zhang, F. & Collins, J. J. CRISPR-based diagnostics. Nat. Biomed. Eng. 5, 643-656 (2021).
29. Li, Y., Li, S., Wang, J. & Liu, G. CRISPR/Cas systems towards next-generation biosensing. Trends Biotechnol. 37, 730-743 (2019).
30. Gootenberg, J. S. et al. Nucleic acid detection with CRISPR-Cas13a/C2c2. Science 356, 438-442 (2017).
31. Chen, J. S. et al. CRISPR-Cas12a target binding unleashes indiscriminate single-stranded DNase activity. Science 360, 436-439 (2018).
32. Joung, J. et al. Detection of SARS-CoV-2 with SHERLOCK one-pot testing. N. Engl. J. Med. 383, 1492-1494 (2020).
33. Li, L. et al. HOLMESv2: a CRISPR-Cas12b-assisted platform for nucleic acid detection and DNA methylation quantitation. ACS Synth. Biol. 8, 2228-2237 (2019).
34. Wang, G., Tian, W., Liu, X., Ren, W. & Liu, C. New CRISPR-derived microRNA sensing mechanism based on Cas12a self-powered and rolling circle transcription-unleashed real-time crRNA recruiting. Anal. Chem. 92, 6702-6708 (2020).
35. Xu, L. Q. et al. Accurate MRSA identification through dual-functional aptamer and CRISPR-Cas12a assisted rolling circle amplification. J. Microbiol. Methods 173, 105917 (2020).
36. Zhang, G., Zhang, L., Tong, J. T., Zhao, X. X. & Ren, J. L. CRISPR-Cas12a enhanced rolling circle amplification method for ultrasensitive miRNA detection. Microchem. J. 158, 105239 (2020).
37. Zhang, M., Wang, H. H., Wang, H., Wang, F. F. & Li, Z. P. CRISPR/Cas12a-assisted ligation-initiated loop-mediated isothermal amplification (CAL-LAMP) for highly specific detection of microRNAs. Anal. Chem. 93, 7942-7948 (2021).
38. Sun, H. H., He, F., Wang, T., Yin, B. C. & Ye, B. C. A Cas12a-mediated cascade amplification method for microRNA detection. Analyst 145, 5547-5552 (2020).
39. Ding, X. et al. Ultrasensitive and visual detection of SARS-CoV-2 using all-in-one dual CRISPR-Cas12a assay. Nat. Commun. 11, 4711 (2020).
40. Li, S. et al. A one-step, one-pot CRISPR nucleic acid detection platform (CRISPR-top): Application for the diagnosis of COVID-19. Talanta 233, 122591 (2021).
41. Lu, S. H. et al. Fast and sensitive detection of SARS-CoV-2 RNA using suboptimal protospacer adjacent motifs for Cas12a. Nat. Biomed. Eng. 6, 286-297 (2022).
42. Bruch, R. et al. CRISPR/Cas13a—powered electrochemical microfluidic biosensor for nucleic acid amplification—free miRNA diagnostics. Adv. Mater. 31, 1970365 (2019).
43. Zheng, F. et al. A highly sensitive CRISPR-empowered surface plasmon resonance sensor for diagnosis of inherited diseases with femtomolar-level real-time quantification. Adv. Sci., e2105231 (2022).
44. Hajian, R. et al. Detection of unamplified target genes via CRISPR-Cas9 immobilized on a graphene field-effect transistor. Nat. Biomed. Eng. 3, 427-437 (2019).
45. Yan, H., Li, Y., Cheng, S. & Zeng, Y. Advances in analytical technologies for extracellular vesicles. Anal. Chem. 93, 4739-4774 (2021).
46. Kalluri, R. & LeBleu, V. S. The biology, function, and biomedical applications of exosomes. Science 367, eaau6977 (2020).
47. Thery, C., Zitvogel, L. & Amigorena, S. Exosomes: composition, biogenesis and function. Nat. Rev. Immunol. 2, 569-579 (2002).
48. Moller, A. & Lobb, R. J. The evolving translational potential of small extracellular vesicles in cancer. Nat. Rev. Cancer 20, 697-709 (2020).
49. van Niel, G., D'Angelo, G. & Raposo, G. Shedding light on the cell biology of extracellular vesicles. Nat. Rev. Mol. Cell Biol. 19, 213-228 (2018).
50. Garcia-Martin, R. et al. MicroRNA sequence codes for small extracellular vesicle release and cellular retention. Nature 601, 446-451 (2022).
51. Anfossi, S., Babayan, A., Pantel, K. & Calin, G. A. Clinical utility of circulating non-coding RNAs—an update. Nat. Rev. Clin. Oncol. 15, 541-563 (2018).
52. Ko, J. et al. miRNA profiling of magnetic nanopore-isolated extracellular vesicles for the diagnosis of pancreatic cancer. Cancer Res. 78, 3688-3697 (2018).
53. Pfeffer, S. R., Yang, C. H. & Pfeffer, L. M. The role of miR-21 in cancer. Drug Dev. Res. 76, 270-277 (2015).
54. Singh, D. et al. Real-time observation of DNA target interrogation and product release by the RNA-guided endonuclease CRISPR Cpf1 (Cas12a). Proc. Natl. Acad. Sci. U.S.A 115, 5444-5449 (2018).
55. Nallur, G. et al. Signal amplification by rolling circle amplification on DNA microarrays. Nucleic Acids Res. 29, E118 (2001).
56. Baner, J., Nilsson, M., Mendel-Hartvig, M. & Landegren, U. Signal amplification of padlock probes by rolling circle replication. Nucleic Acids Res. 26, 5073-5078 (1998).
57. Dahl, F. et al. Circle-to-circle amplification for precise and sensitive DNA analysis. Proc. Natl. Acad. Sci. U.S.A 101, 4548-4553 (2004).
58. Ali, M. M. et al. Rolling circle amplification: a versatile tool for chemical biology, materials science and medicine. Chem. Soc. Rev. 43, 3324-3341 (2014).
59. Lohman, G. J., Zhang, Y., Zhelkovsky, A. M., Cantor, E. J. & Evans, T. C., Jr. Efficient DNA ligation in DNA-RNA hybrid helices by Chlorella virus DNA ligase. Nucleic Acids Res. 42, 1831-1844 (2014).
60. Jin, J. M., Vaud, S., Zhelkovsky, A. M., Posfai, J. & McReynolds, L. A. Sensitive and specific miRNA detection method using SplintR Ligase. Nucleic Acids Res. 44 (2016).
61. Wee, E. J. H. & Trau, M. Simple Isothermal Strategy for Multiplexed, Rapid, Sensitive, and Accurate miRNA Detection. ACS sensors 1, 670-675 (2016).
62. Lagunavicius, A., Kiveryte, Z., Zimbaite-Ruskuliene, V., Radzvilavicius, T. & Janulaitis, A. Duality of polynucleotide substrates for Phi29 DNA polymerase: 3′->5′ RNase activity of the enzyme. RNA 14, 503-513 (2008).
63. Bloomston, M. et al. MicroRNA expression patterns to differentiate pancreatic adenocarcinoma from normal pancreas and chronic pancreatitis. JAMA 297, 1901-1908 (2007).
64. Goto, T. et al. An elevated expression of serum exosomal microRNA-191,-21,-451a of pancreatic neoplasm is considered to be efficient diagnostic marker. BMC Cancer 18, 116 (2018).
65. Que, R., Ding, G., Chen, J. & Cao, L. Analysis of serum exosomal microRNAs and clinicopathologic features of patients with pancreatic adenocarcinoma. World J. Surg. Oncol. 11, 219 (2013).
66. Roldo, C. et al. MicroRNA expression abnormalities in pancreatic endocrine and acinar tumors are associated with distinctive pathologic features and clinical behavior. J. Clin. Oncol. 24, 4677-4684 (2006).
67. Xu, Y. F., Hannafon, B. N., Zhao, Y. D., Postier, R. G. & Ding, W. Q. Plasma exosome miR-196a and miR-1246 are potential indicators of localized pancreatic cancer. Oncotarget 8, 77028-77040 (2017).
68. Szafranska, A. E. et al. MicroRNA expression alterations are linked to tumorigenesis and non-neoplastic processes in pancreatic ductal adenocarcinoma. Oncogene 26, 4442-4452 (2007).
69. Zhang, Y. et al. Profiling of 95 microRNAs in pancreatic cancer cell lines and surgical specimens by real-time PCR analysis. World J. Surg. 33, 698-709 (2009).
70. Vicentini, C. et al. Exosomal miRNA signatures of pancreatic lesions. BMC Gastroenterol. 20, 137 (2020).
71. Takahasi, K. et al. Usefulness of exosome-encapsulated microRNA-451a as a minimally invasive biomarker for prediction of recurrence and prognosis in pancreatic ductal adenocarcinoma. J. Hepatobiliary Pancreat. Sci. 25, 155-161 (2018).
72. Kawamura, S. et al. Exosome-encapsulated microRNA-4525, microRNA-451a and microRNA-21 in portal vein blood is a high-sensitive liquid biomarker for the selection of high-risk pancreatic ductal adenocarcinoma patients. J. Hepatobiliary Pancreat. Sci. 26, 63-72 (2019).
73. Madhavan, B. et al. Combined evaluation of a panel of protein and miRNA serum-exosome biomarkers for pancreatic cancer diagnosis increases sensitivity and specificity. Int. J. Cancer 136, 2616-2627 (2015).
74. Zhang, P., He, M. & Zeng, Y. Ultrasensitive microfluidic analysis of circulating exosomes using a nanostructured graphene oxide/polydopamine coating. Lab Chip 16, 3033-3042 (2016).
75. Verel-Yilmaz, Y. et al. Extracellular vesicle-based detection of pancreatic cancer. Front. Cell. Dev. Biol. 9, 697939 (2021).
76. Xu, Y. F. et al. Isolation of extra-cellular vesicles in the context of pancreatic adenocarcinomas: Addition of one stringent filtration step improves recovery of specific microRNAs. PLoS One 16, e0259563 (2021).
77. Mou, G., Wang, K., Xu, D. & Zhou, G. Evaluation of three RT-qPCR-based miRNA detection methods using seven rice miRNAs. Biosci. Biotechnol. Biochem. 77, 1349-1353 (2013).
78. Andreu, Z. et al. Comparative analysis of EV isolation procedures for miRNAs detection in serum samples. J. Extracell. Vesicles 5, 31655 (2016).
79. Brennan, K. et al. A comparison of methods for the isolation and separation of extracellular vesicles from protein and lipid particles in human serum. Sci. Rep. 10, 1039 (2020).
80. Gouin, K. et al. A comprehensive method for identification of suitable reference genes in extracellular vesicles. J. Extracell. Vesicles 6, 1347019 (2017).
81. Ragni, E. et al. miRNA Reference Genes in Extracellular Vesicles Released from Amniotic Membrane-Derived Mesenchymal Stromal Cells. Pharmaceutics 12 (2020).
82. Cai, S. et al. Single-molecule amplification-free multiplexed detection of circulating microRNA cancer biomarkers from serum. Nat. Commun. 12, 3515 (2021).
83. Veryaskina, Y. A. et al. Selection of reference genes for quantitative analysis of microRNA expression in three different types of cancer. PLoS One 17, e0254304 (2022).
84. Xiang, M. et al. U6 is not a suitable endogenous control for the quantification of circulating microRNAs. Biochem. Biophys. Res. Commun. 454, 210-214 (2014).
85. Lou, G. et al. Differential distribution of U6 (RNU6-1) expression in human carcinoma tissues demonstrates the requirement for caution in the internal control gene selection for microRNA quantification. Int. J. Mol. Med. 36, 1400-1408 (2015).
86. Yee, N. S., Zhang, S., He, H. Z. & Zheng, S. Y. Extracellular vesicles as potential biomarkers for early detection and diagnosis of pancreatic cancer. Biomedicines 8, 581 (2020).
87. Gootenberg, J. S. et al. Multiplexed and portable nucleic acid detection platform with Cas13, Cas12a, and Csm6. Science 360, 439-444 (2018).
88. Wang, N. et al. Recent advances in the rapid detection of microRNA with lateral flow assays. Biosens. Bioelectron. 211, 114345 (2022).
89. Cheng, Y. et al. Highly sensitive determination of microRNA using target-primed and branched rolling-circle amplification. Angew. Chem. 121, 3318-3322 (2009).
90. Liu, H. et al. High specific and ultrasensitive isothermal detection of microRNA by padlock probe-based exponential rolling circle amplification. Anal. Chem. 85, 7941-7947 (2013).
91. Weber, J. A. et al. The microRNA spectrum in 12 body fluids. Clin Chem 56, 1733-1741 (2010).
92. Bhome, R. et al. Exosomal microRNAs (exomiRs): Small molecules with a big role in cancer. Cancer Lett. 420, 228-235 (2018).
93. Salehi, M. & Sharifi, M. Exosomal miRNAs as novel cancer biomarkers: Challenges and opportunities. J. Cell. Physiol. 233, 6370-6380 (2018).
94. O'Brien, K., Breyne, K., Ughetto, S., Laurent, L. C. & Breakefield, X. O. RNA delivery by extracellular vesicles in mammalian cells and its applications. Nat. Rev. Mol. Cell Biol. 21, 585-606 (2020).
95. Chevillet, J. R. et al. Quantitative and stoichiometric analysis of the microRNA content of exosomes. Proc. Natl. Acad. Sci. U.S.A 111, 14888-14893 (2014).
96. Krepelkova, I. et al. Evaluation of miRNA detection methods for the analytical characteristics necessary for clinical utilization. Biotechniques 66, 277-284 (2019).
97. Tian, W., Liu, X., Wang, G. & Liu, C. A hyperbranched transcription-activated CRISPR-Cas12a signal amplification strategy for sensitive microRNA sensing. Chem. Commun. 56, 13445-13448 (2020).
98. Tian, H. et al. Precise Quantitation of MicroRNA in a Single Cell with Droplet Digital PCR Based on Ligation Reaction. Anal. Chem. 88, 11384-11389 (2016).
99. Roy, S., Soh, J. H. & Ying, J. Y. A microarray platform for detecting disease-specific circulating miRNA in human serum. Biosens. Bioelectron. 75, 238-246 (2016).
100. Chen, K., Wang, Q., Kornmann, M., Tian, X. & Yang, Y. The role of exosomes in pancreatic cancer from bench to clinical application: an updated review. Front. Oncol. 11, 644358 (2021).
101. Park, W., Chawla, A. & O'Reilly, E. M. Pancreatic cancer: a review. JAMA 326, 851-862 (2021).
102. Wong, M. C. S. et al. Global temporal patterns of pancreatic cancer and association with socioeconomic development. Sci. Rep. 7, 3165 (2017).
103. Brezgyte, G., Shah, V., Jach, D. & Crnogorac-Jurcevic, T. Non-invasive biomarkers for earlier detection of pancreatic cancer-a comprehensive review. Cancers 13, 2722 (2021).
104. Yang, J. S. et al. Early screening and diagnosis strategies of pancreatic cancer: a comprehensive review. Cancer Commun. 41, 1257-1274 (2021).
105. Huang, L. et al. Resection of pancreatic cancer in Europe and USA: an international large-scale study highlighting large variations. Gut 68, 130-139 (2019).
106. Siegel, R. L., Miller, K. D. & Jemal, A. Cancer statistics, 2018. CA-Cancer J. Clin. 68, 7-30 (2018).
107. Ballehaninna, U. K. & Chamberlain, R. S. The clinical utility of serum CA 19-9 in the diagnosis, prognosis and management of pancreatic adenocarcinoma: An evidence based appraisal. J. Gastrointest. Oncol. 3, 105-119 (2012).
108. Zhang, Y. et al. Tumor markers CA19-9, CA242 and CEA in the diagnosis of pancreatic cancer: a meta-analysis. Int. J. Clin. Exp. Med. 8, 11683-11691 (2015).
109. Zhang, P. et al. Ultrasensitive detection of circulating exosomes with a 3D-nanopatterned microfluidic chip. Nat. Biomed. Eng. 3, 438-451 (2019).
110. Zhang, P. et al. Molecular and functional extracellular vesicle analysis using nanopatterned microchips monitors tumor progression and metastasis. Sci. Transl. Med. 12, eaaz2878 (2020).
111. Chandrasekaran, S. S. et al. Rapid detection of SARS-CoV-2 RNA in saliva via Cas13. Nat. Biomed. Eng. 6, 944-956 (2022).
112. Zhang, T. et al. A paper-based assay for the colorimetric detection of SARS-CoV-2 variants at single-nucleotide resolution. Nat. Biomed. Eng. (2022).
113. Niemeyer, C. M., Adler, M. & Wacker, R. Detecting antigens by quantitative immuno-PCR. Nat. Protoc. 2, 1918-1930 (2007).
114. Li, L. et al. Nucleic acid aptamers for molecular diagnostics and therapeutics: advances and perspectives. Angew. Chem.—Int. Edit. 60, 2221-2231 (2021).
115. Han, S. et al. Primary outgrowth cultures are a reliable source of human pancreatic stellate cells. Lab. Invest. 95, 1331-1340 (2015).
116. Han, S. et al. The proteome of pancreatic cancer-derived exosomes reveals signatures rich in key signaling pathways. Proteomics 19, e1800394 (2019).
117. Tibshirani, R. Regression shrinkage and selection via the Lasso. J. R. Stat. Soc. Ser. B-Stat. Methodol. 58, 267-288 (1996).
118. Homrighausen, D. & McDonald, D. J. Leave-one-out cross-validation is risk consistent for lasso. Mach. Learn. 97, 65-78 (2014).

### Example 2

### Padlock Design and Sequences for Embodiments of Padlock Probes and Components of Reaction

The padlock probes of the present disclosure, such as described in Example 1 above can be pre-designed and/or customized for systems and methods of the present disclosure. As illustrated in FIG. 1A, the padlock contains a ligation zone (medium gray), a detection zone (black) and the rest sequences (light gray). As described above, the ligation zone is used for the capture of target miRNA, while the detection zone is used for the activation of RNP, and the rest sequence is used for keeping the length of the padlock probe to minimize the rigidity of the circular padlock and to adjust the secondary structure of the padlock probe.

The ligation zone locates at the 5′-end and 3′-end of the padlock probe, and the 5′-end is phosphorylated. The detection zone can be located at any position (in the right, middle or left) flanked by the two parts of ligation sequences. For instance, variations illustrated in FIG. 1 (i), (ii), and (iii) were designed with the detection zone in different configurations. See Table 2, below, including 4 different padlock sequence configurations: padlock 1 (SEQ ID NO: 1), padlock 2 (SEQ ID NO: 2), padlock 3 (SEQ ID NO: 3), and padlock 4 (SEQ ID NO: 4). For each of the sequences, the ligation zone is shown in bold with bold underlining, and the detection zone is shown with single underlining. The rest sequence has no underlining.

The ligation zone contains the sequence that are complementary to the target miRNA, and the nick in the padlock can be located at any position of the miRNA as long as the hybridization is stable (Tm>37° C.). The detection zone is the sequence containing complementary sequences that can hybridize/couple with a crRNA of the RNP complex. Although PAM sequences are often needed for binding double stranded DNA CRISPR targets, since the padlock is single stranded, as described in Example 1, it was found that the system performs better in the absence of a PAM sequence in the detection zone, and thus for embodiments, a PAM sequence (e.g., TTTV, where V=A, G, C) is NOT added to the 5′ end of the detection zone.

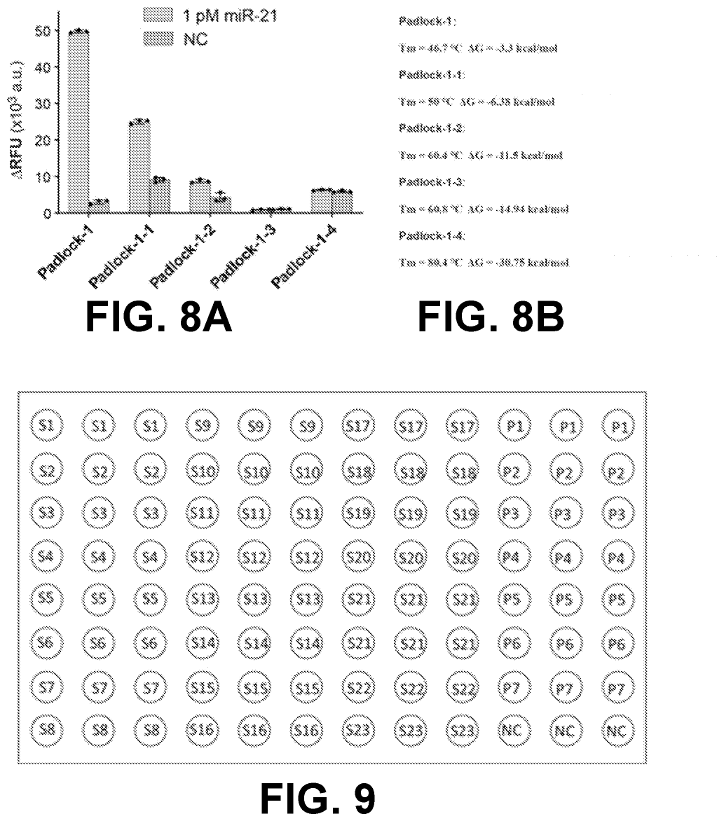

The total length of the padlock probe can from about 40-100 nt including the rest sequences. Secondary structures should be eliminated by adjusting the rest sequence, as secondary structures were found to affect performance, as described below. The sequence highlighted in the red rectangular were modified to induce different secondary structures. The padlock sequences 1-1 (SEQ ID NO: 5), 1-2 (SEQ ID NO: 6), 1-3 (SEQ ID NO: 7), and 1-4 (SEQ ID NO: 8) were designed as variations of padlock 1 (SEQ ID 1). The portions of the sequences in bold are mutated and can pair with the regions with dashed underlining to form hairpin structures. The one-pot reactions were performed with 1 pM miR-21 in triplicate and results are shown in FIG. 8A. Error bars indicate one S.D. (n=3). The DNA sequences were analyzed with the OligoAnalyzer (IDT) and Tm and ΔG values of the most stable hairpin structure are shown (FIG. 8B).

As described in Example 1, the amounts of various components of the assay were optimized. Table 3, below, provides the recipes used and optimized for each of the 4 target miRNAs tested in Example 1, and Table 4 describes the optimized ranges and preferred amounts for the various reaction components according to some embodiments of the methods and systems for EXTRA-CRISPR of the present disclosure. Finally, the buffer used in the examples of the present disclosure is from an NEB product and the composition is as follows: 1× SplintR Ligase Reaction Buffer, about 50 mM Tris-HCl, about 10 mM MgCl2, about 1 mM ATP, and about 10 mM DTT at a pH of about 7.5 and a temperature of about 25° C.

### Sequences:

The following list and tables provide sequences of nucleic acids and polypeptides described in the present disclosure and/or used in the Example.

TABLE 2
List of nucleic acid sequences used in Examples 1 and 2

| DNA Sequences | sequence |
|---|---|
| Padlock-1 | 5′p-CTGATAAGCTAAGATACCCTAACCATCGATCGTCGCCGTCCAG |
| SEQ ID NO: 1 | CTCGACCTCAACATCAGT |
| Padlock-2 | 5′p-CTGATAAGCTAAGATACCCTAACCATCGATTTTACGTCGCCGT |
| SEQ ID NO: 2 | CCAGCTCGACCTCAACATCAGT |
| Padlock-3 | 5′p-CTGATAAGCTACGTCGCCGTCCAGCTCGACCAGATACCCTAA |
| SEQ ID NO: 3 | CCATCGATTCAACATCAGT |
| Padlock-4 | 5′p-CTGATAAGCTAAGATACCCTCGTCGCCGTCCAGCTCGACCAA |
| SEQ ID NO: 4 | CCATCGATTCAACATCAGT |
| Padlock 1-1 | 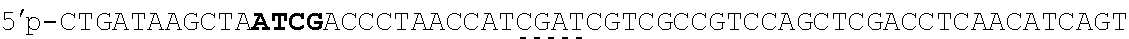 |
| SEQ ID NO: 5 | |
| Padlock 1-2 | 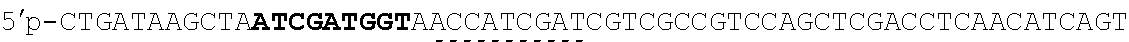 |
| SEQ ID NO: 6 | |
| Padlock 1-3 | 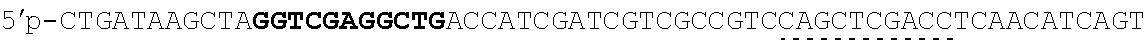 |
| SEQ ID NO: 7 | |
| Padlock 1-4 | 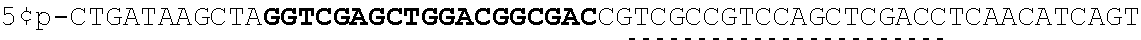 |
| SEQ ID NO: 8 | |
| Padlock-miR-21 | 5′p- CTGATAAGCTAAGATACCCTAACCATCGATCGTCGCCGTCCAG |
| SEQ ID NO: 9 | CTCGACCTCAACATCAGT |
| Padlock-miR-196a | 5′p-GAAACTACCTAAGATACCCTAACCATCGATCGTCGCCGTCCAG |
| SEQ ID NO: 10 | CTCGACCCCCAACAACAT |
| Padlock-miR-451a | 5′p-GGTAACGGTTTAGATACCCTAACCATCGATCGTCGCCGTCCA |
| SEQ ID NO: 11 | GCTCGACCAACTCAGTAAT |
| Padlock-miR-1246 | 5′p-AAATCCATTAGATACCCTAACCATCGATCGTCGCCGTCCAGCT |
| SEQ ID NO: 12 | CGACCCCTGCTCCAA |
| Synthetic short cut | GGTCGAGCTGGACGGCGACGATCGATGGTTAGGGTATCTTAGCTT |
| SEQ ID NO: 13 | ATCAGACTGATGTTGA |
| Reporter | 5-FAM-TTATT-IABKFQ-3′ |
| RNA sequences | Sequence (SEQ ID NO) |
| miR-21 | UAGCUUAUCAGACUGAUGUUGA (SEQ ID NO: 14) |
| miR-21-mismatch1 | UAGCUUAUCAUACUGAUGUUGA (SEQ ID NO: 15) |
| miR-17 | CAAAGUGCUUACAGUGCAGGUAG (SEQ ID NO: 16) |
| miR-18a | UAAGGUGCAUCUAGUGCAGAUAG (SEQ ID NO: 17) |
| miR-106a | AAAAGUGCUUACAGUGCAGGUAG (SEQ ID NO: 18) |
| miR-145 | GUCCAGUUUUCCCAGGAAUCCCU (SEQ ID NO: 19) |
| miR-196a | UAGGUAGUUUCAUGUUGUUGGG (SEQ ID NO: 20) |
| miR-205 | UCCUUCAUUCCACCGGAGUCUG (SEQ ID NO: 21) |
| miR-451a | AAACCGUUACCAUUACUGAGUU (SEQ ID NO: 22) |
| miR-1246 | AAUGGAUUUUUGGAGCAGG (SEQ ID NO: 23) |
| crRNA | UAAUUUCUACUAAGUGUAGAUCGUCGCCGUCCAGCUCGACC |
| | (SEQ ID NO: 24) |

TABLE 3
Summary of the recipes for EXTRA-CRISPR detection of four miRNAs in EX 1.

| Components | | | | | | | | |
|---|---|---|---|---|---|---|---|---|
| (μL) | miR-21 | | miR-196 | | miR451a | | miR-1246 | |
| Buffer (10×) | 2 | μL | 2 | μL | 2 | μL | 2 | μL |
| dNTP | 400 | nM | 400 | nM | 400 | nM | 400 | nM |
| BSA | 0.2 | mg/mL | 0.2 | mg/mL | 0.2 | mg/mL | 0.2 | mg/mL |
| Padlock | 100 | nM | 100 | nM | 10 | nM | 10 | nM |
| miRNA | 2 | μL | 2 | μL | 2 | μL | 2 | μL |
| Phi29 | 0.1 | U/μL | 0.1 | U/μL | 0.05 | U/μL | 0.05 | U/μL |
| SplintR | 0.625 | U/μL | 1.25 | U/μL | 1.25 | U/μL | 1.25 | U/μL |
| RNP | 1 | nM | 1 | nM | 1 | nM | 1 | nM |
| Reporter | 1 | μM | 2 | μM | 4 | μM | 1 | μM |

| | |
|---|---|
| Water | Adjust water volume accordingly (total reaction volume: 20 μL). |

TABLE 4
Summary of the recipes for EXTRA-CRISPR detection miRNAs

| Components | | |
|---|---|---|
| (μL) | Optimal | Range |

| Buffer (10×) | 2 | μL | 2 | μL |
|---|---|---|---|---|
| dNTP | 400 | nM | 100-1000 | nM |
| BSA | 0.2 | mg/mL | 0-2 | mg/mL |
| Padlock | 100 | nM | 5-500 | nM |
| miRNA | 2 | μL | adjustable | |
| Phi29 | 0.1 | U/μL | 0.01-5 | U/μL |
| SplintR | 0.625 | U/μL | 0.01-5 | U/μL |
| RNP | 1 | nM | 0.1-10 | nM |
| Reporter | 1 | μM | 0.1-10 | μM |

| | |
|---|---|
| Water | Adjust water volume accordingly |
| | (total reaction volume: 20 μL). |

### Example 3

The present example describes the design of a customized multi-well plate for detecting multiple target polynucleotides in a sample and/or in multiple samples. In embodiments, the plate can be a multi-well plate, such as a 96-well plate with V-shaped wells like a PCR tube. Each reaction well is pre-loaded with a lyophilized pellet including lyophilized reaction components. The plate with the pre-loaded pellets can be sealed with a film, such as a plastic or foil membrane and contained in a package for shipping and storage.

The pellets can contain the following lyophilized components:
a) a lyophilized Cas12a CRISPR-associated (Cas) enzyme-crRNA complex; b) a lyophilized ligase; c) a lyophilized polymerase; d) and one or more of the following additional components: lyophilized buffer, BSA, dNTP, reporter, specific/customized padlock probes. Before use, the customer can peel off the seal and add 20 uL liquid sample containing targets to dissolve the pellet, after that, each well can be sealed with caps and put it in the PCR machine or other incubator for the isothermal reaction and signal monitoring.

The plate can be designed for the detection of one specific target miRNA from multiple samples, it can also be designed for the detection of multiple different target miRNAs from one sample. FIG. 9 illustrates a demo configuration design of the 96-well plate to detect miR-21 from 23 samples. S1-S23 are 23 samples, and each sample is conducted in triplicate. P1-P7 are positive control of miR-21 with different concentrations for the establishment of standard curve. NC is the negative control.

Compared with commercial miRNA detection by RT-qPCR, this method doesn't need liquid transfer/pipetting, such as required between RT and PCR, so combining the 96-well plate design, one-step multiplex miRNA screening can be done. Besides the kit described in the present example, this customized product provides an alternative for multiplex miRNA detection/screening in scientific research and clinic diagnostics, simplifying the protocol for miRNA detection. An illustration of an example multi-well plate for EXTRA-CRISPR is shown in FIG. 9

### Example 4

The enzyme Cas12a in the present methods, systems, and kits is an endonuclease. It has an indiscriminate single-strand DNase activity (trans-cleavage) after being activated. Many CRISPR-cas12a based nucleic acid detection technologies used its trans-cleavage property. In the present matter, when simply mixing the three enzymatic reactions into one pot, the cas12a will not only generate signals by cleaving reporters, but also single-stranded padlock sequences and even padlock-RCA product duplex will be cleaved by trans- and cis-cleavage, respectively. Hence, according to intuition and inference, the combination of the three enzymes (Cas12a, ligase, and polymerase) with padlock sequences with or without target could lead to the cease of the reaction as the materials for ligation and RCA are prone to be destroyed by cas12a.

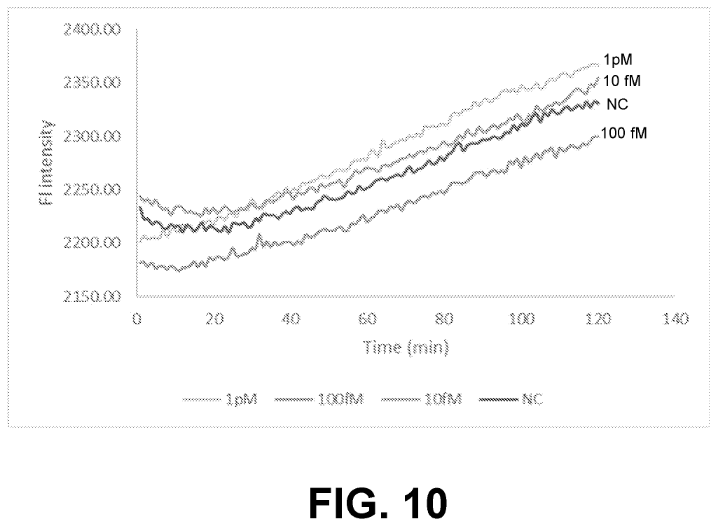

In the present study, to demonstrate superiority and unexpected results of the methods and systems of the present disclosure, a simple combination was tested where Cas12a (RNP) concentration is 50 nM which is the most commonly used concentration in ca12a detection methods (Chen, J. S. et al. CRISPR-Cas12a target binding unleashes indiscriminate single-stranded DNase activity. Science 360, 436-439, doi:10.1126/science.aar6245 (2018)). The concentration of both Phi29 and SplintR were 0.1 U/μL and other components were the same as those in Table 4, above. As shown in FIG. 10, one-pot reaction by simply mixing the three enzymes and other reaction components generates no signals even when detecting a high concentration of target miRNA-21 (1 pM; no difference from negative control (NC)). This result demonstrates that the present hypothesis and explanation above are correct, and the one-pot reaction should not feasible by simply mixing three enzymatic reactions according to other methods or at standard concentrations. However, by designing and optimizing the reaction according to methods, systems, and kits as described herein, an extremely strong signal can be detected with even just 1 pM of miR-21 (FIG. 10), which would not have thought as being possible based on previous methods (such as those of Chen et al, cited above).

1. A method of detecting a target polynucleotide in a sample, the method comprising:
incubating the contents of a reaction vessel at a first isothermal temperature for a first period of time, the reaction vessel comprising:
a sample comprising one or more microRNAs;
a padlock probe comprising a ligation zone and a detection zone, the ligation zone comprising a polynucleotide sequence complementary to a microRNA target of interest, and the detection zone comprising a sequence capable of hybridizing with a crRNA, the ligation zone of the padlock probe comprising a nick in the sequence of the ligation zone complementary to the miRNA target of interest, wherein the nick defines the 5′ and 3′ ends of the padlock probe, and wherein the 5′ end is phosphorylated;
a ribonucleoprotein complex (RNP) comprising a Cas12a CRISPR-associated (Cas) enzyme and a CRISPR-RNA (crRNA), wherein the crRNA is capable of hybridizing with the detection zone of the padlock probe;
a ligase;
a polymerase; and
a reporter deoxyribonucleic acid (DNA) capable of producing a CRISPR-generated detectable signal or detectable molecule upon cleavage by the Cas enzyme; and
detecting the CRISPR-generated detectable signal or detectable molecule if a target microRNA is present in the sample.

2. The method of claim 1, further comprising providing the sample, padlock probe, RNP, ligase, polymerase, and reporter DNA in a single reaction vessel prior to incubating the vessel at a first temperature.

3. The method of claim 1, wherein the first isothermal temperature is about 16 to about 48° C.

4. The method of claim 1, wherein the first period of time is about 10 min to about 3 hours.

5. The method of claim 1, further comprising combining the sample and the padlock probe in the reaction vessel and incubating the sample and the padlock probe at a second temperature for a second period of time before adding RNP, ligase, polymerase, and reporter DNA and incubating at the first temperature and first period of time in the reaction vessel.

6. The method of claim 5, wherein the second temperature is about 45 to about 99° C.

7. The method of claim 1, wherein the second period of time is about 1 to about 30 minutes.

8. The method of claim 1, wherein the polynucleotide sequence of the detection zone lacks a PAM sequence.

9. The method of claim 1, wherein the cas12a enzyme is a Lba cas12a (cpf1).

10. The method of claim 1, wherein the crRNA comprises a polynucleotide sequence complementary to a conserved sequence of Lba cas12a, a variable sequence of Lba cas12a, or both.

11. The method of claim 1, wherein the ligase is a SplintR ligase or T4 ligase.

12. The method of claim 1, wherein the polymerase is a Phi29 polymerase, Bst polymerase, or a Klenow Fragment.

13. The method of claim 1, wherein the reporter DNA comprises: a polynucleotide, a detectable molecule, and a quencher, wherein the detectable molecule and quencher are linked to opposite ends of the polynucleotide and wherein the polynucleotide is configured to be cleaved by the Cas enzyme of the RNP upon activation.

14. The method of claim 1, wherein the detectable molecule is one or more of FAM, FITC, Cy3, Cy5, HEX, TAMRA.

15. The method of claim 1, wherein the reaction vessel further comprises one or more additional components selected from the group consisting of: deoxyribonucleoside triphosphates (dNTPs), bovine serum albumin (BSA), a reaction buffer, water, and combinations thereof.

16. The method of claim 1, wherein the sample is from a subject having or suspected of having a pancreatic ductal adenocarcinoma (PDAC) and wherein the one or more microRNAs are one or more biomarkers of PDAC.

17. The method of claim 1, wherein the concentration of RNP is from about 0.1-10 nM.

18. The method of claim 1, wherein the RNP is formed by incubating the cas12a enzyme and crRNA at a third temperature for a third period of time before placing in the reaction vessel.

19. The method of claim 18, wherein the third temperature is about 4 to about 48° C. and the third period of time is about 10 to about 180 minutes.

20. A one-pot nucleic acid detection system for detecting a target polynucleotide in a sample, the system comprising:
a set of isothermal detection components comprising:
a Cas12a CRISPR-associated (Cas) enzyme;
a ligase;
a polymerase; and
a reporter deoxyribonucleic acid (DNA) capable of producing a CRISPR-generated detectable signal or detectable molecule upon cleavage by the Cas enzyme.

21.-54. (canceled)
